SEQUENCE LISTING (143 sequences)

SEQ ID NO: 1 - RNA, 49 nt, synthetic construct
  misc_feature 1..49: note = Synthetic sequence
  source 1..49: mol_type = other RNA
tccccaatcgtggcgtgtcggcctgcttcggcaggcactggcgccggga

SEQ ID NO: 2 - RNA, 101 nt, synthetic construct
  misc_feature 1..101: note = Synthetic sequence
  source 1..101: mol_type = other RNA
ttgccatgtgtatgtgggttcgcccacatactctgatgatccccaatcgtggcgtgtcggcctgcttcggcaggcactggcgccgggatcattcatggcaa

SEQ ID NO: 3 - RNA, 114 nt, synthetic construct
  misc_feature 1..114: note = Synthetic sequence
  source 1..114: mol_type = other RNA
gcccggatagctcagtcggtagagcagcgctgccaatcgtggcgtgtcggcctgcttcggcaggcactggcgccgcagcgcgggtccagggttcaagtccctgttcgggcgcca

SEQ ID NO: 4 - RNA, 105 nt, synthetic construct
  misc_feature 1..105: note = Synthetic sequence
  source 1..105: mol_type = other RNA
ttgccatgtgtatgtgggttcgcccacatactctgatgatccagacggtcgggtccagatattcgtatctgtcgagtagagtgtgggctggatcattcatggcaa

SEQ ID NO: 5 - RNA, 234 nt, synthetic construct
  misc_feature 1..234: note = Synthetic sequence
  source 1..234: mol_type = other RNA
ttgccatgtgtatgtgggagacggtcgggtccatctgagacggtcgggtccagatattcgtatctgtcgagtagagtgtgggctcagatgtcgagtagagtgtgggctcccacatactctgatgatccagacggtcgggtccatctgagacggtcgggtccagatattcgtatctgtcgagtagagtgtgggctcagatgtcgagtagagtgtgggctggatcattcatggcaa

SEQ ID NO: 6 - RNA, 53 nt, synthetic construct
  misc_feature 1..53: note = Synthetic sequence
  source 1..53: mol_type = other RNA
ggcccccaatcgtggcgtgtcggcctgcttcggcaggcactggcgccggggcc

SEQ ID NO: 7 - RNA, 55 nt, synthetic construct
  misc_feature 1..55: note = Synthetic sequence
  source 1..55: mol_type = other RNA
ggccccccaatcgtggcgtgtcggcctgcttcggcaggcactggcgccgggggcc

SEQ ID NO: 8 - RNA, 144 nt, synthetic construct
  misc_feature 1..144: note = Synthetic sequence
  source 1..144: mol_type = other RNA
ttgccatgtgtatgtgggttcgcccacatactctgatgatccccaatcgtggcgtgtcggcctctcccaatcgtggcgtgtcggcctctcttcggagaggcactggcgccggagaggcactggcgccgggatcattcatggcaa

SEQ ID NO: 9 - RNA, 226 nt, synthetic construct
  misc_feature 1..226: note = Synthetic sequence
  source 1..226: mol_type = other RNA
ttgccatgtgtatgtgggttcgcccacatactctgatgatccccaatcgtggcgtgtcggcctctcccaatcgtggcgtgtcggcctctcccaatcgtggcgtgtcggcctctcccaatcgtggcgtgtcggcctctcttcggagaggcactggcgccggagaggcactggcgccggagaggcactggcgccggagaggcactggcgccgggatcattcatggcaa

SEQ ID NO: 10 - RNA, 390 nt, synthetic construct
  misc_feature 1..390: note = Synthetic sequence
  source 1..390: mol_type = other RNA
ttgccatgtgtatgtgggttcgcccacatactctgatgatccccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcttcggagaggcactggcgccggagaggcactggcgccggagaggcactggcgccggagaggcactggcgccggagaggcactggcgccggagaggcactggcgccggagaggcactggcgccggagaggcactggcgccgggatcattcatggcaa

SEQ ID NO: 11 - RNA, 718 nt, synthetic construct
  misc_feature 1..718: note = Synthetic sequence
  source 1..718: mol_type = other RNA
ttgccatgtgtatgtgggttcgcccacatactctgatgatccccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcttcggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccgggatcattcatggcaa

SEQ ID NO: 12 - RNA, 1374 nt, synthetic construct
  misc_feature 1..1374: note = Synthetic sequence
  source 1..1374: mol_type = other RNA
ttgccatgtgtatgtgggttcgcccacatactctgatgatccccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcccaatcgtggcgtgtcggcctctcttcggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccggagaggcactggcgccgggatcattcatggcaa

SEQ ID NO: 13 - RNA, 177 nt, synthetic construct
  misc_feature 1..177: note = Synthetic sequence
  source 1..177: mol_type = other RNA
ctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggcc

SEQ ID NO: 14 - RNA, 354 nt, synthetic construct
  misc_feature 1..354: note = Synthetic sequence
  source 1..354: mol_type = other RNA
ctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggcc

SEQ ID NO: 15 - RNA, 708 nt, synthetic construct
  misc_feature 1..708: note = Synthetic sequence
  source 1..708: mol_type = other RNA
ctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggcc

SEQ ID NO: 16 - RNA, 1416 nt, synthetic construct
  misc_feature 1..1416: note = Synthetic sequence
  source 1..1416: mol_type = other RNA
ctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggccctgcaggtcgactctagaaaggcccccaatcgtggcgtgtcggcctgcttcggcaggcactggcgccggggccctgcagtattcccgggttcattagatcctaaggtacctaattgcctagaaaggcccccaatcgtggcgtgtcggcctgcttcggcaggcactggcgccggggcc

SEQ ID NO: 17 - RNA, 263 nt, synthetic construct
  misc_feature 1..263: note = Synthetic sequence
  source 1..263: mol_type = other RNA
ctgcaggtcgactctagaaaggcccccaatcgtggcgtgtcggcctctcccaatcgtggcgtgtcggcctctcttcggagaggcactggcgccggagaggcactggcgccggggccctgcagtattcccgggttcattagatcctaaggtacctaattgcctagaaaggcccccaatcgtggcgtgtcggcctctcccaatcgtggcgtgtcggcctctcttcggagaggcactggcgccggagaggcactggcgccggggcc

SEQ ID NO: 18 - RNA, 526 nt, synthetic construct
  misc_feature 1..526: note = Synthetic sequence
  source 1..526: mol_type = other RNA
ctgcaggtcgactctagaaaggcccccaatcgtggcgtgtcggcctctcccaatcgtggcgtgtcggcctctcttcggagaggcactggcgccggagaggcactggcgccggggccctgcagtattcccgggttcattagatcctaaggtacctaattgcctagaaaggcccccaatcgtggcgtgtcggcctctcccaatcgtggcgtgtcggcctctcttcggagaggcactggcgccggagaggcactggcgccggggccctgcaggtcgactctagaaaggcccccaatcgtggcgtgtcggcctctcccaatcgtggcgtgtcggcctctcttcggagaggcactggcgccggagaggcactggcgccggggccctgcagtattcccgggttcattagatcctaaggtacctaattgcctagaaaggcccccaatcgtggcgtgtcggcctctcccaatcgtggcgtgtcggcctctcttcggagaggcactggcgccggagaggcactggcgccggggcc

SEQ ID NO: 19 - RNA, 1052 nt, synthetic construct
  misc_feature 1..1052: note = Synthetic sequence
  source 1..1052: mol_type = other RNA
ctgcaggtcgactctagaaaggcccccaatcgtggcgtgtcggcctctcccaatcgtggcgtgtcggcctctcttcggagaggcactggcgccggagaggcactggcgccggggccctgcagtattcccgggttcattagatcctaaggtacctaattgcctagaaaggcccccaatcgtggcgtgtcggcctctcccaatcgtggcgtgtcggcctctcttcggagaggcactggcgccggagaggcactggcgccggggccctgcaggtcgactctagaaaggcccccaatcgtggcgtgtcggcctctcccaatcgtggcgtgtcggcctctcttcggagaggcactggcgccggagaggcactggcgccggggccctgcagtattcccgggttcattagatcctaaggtacctaattgcctagaaaggcccccaatcgtggcgtgtcggcctctcccaatcgtggcgtgtcggcctctcttcggagaggcactggcgccggagaggcactggcgccggggccctgcaggtcgactctagaaaggcccccaatcgtggcgtgtcggcctctcccaatcgtggcgtgtcggcctctcttcggagaggcactggcgccggagaggcactggcgccggggccctgcagtattcccgggttcattagatcctaaggtacctaattgcctagaaaggcccccaatcgtggcgtgtcggcctctcccaatcgtggcgtgtcggcctctcttcggagaggcactggcgccggagaggcactggcgccggggccctgcaggtcgactctagaaaggcccccaatcgtggcgtgtcggcctctcccaatcgtggcgtgtcggcctctcttcggagaggcactggcgccggagaggcactggcgccggggccctgcagtattcccgggttcattagatcctaaggtacctaattgcctagaaaggcccccaatcgtggcgtgtcggcctctcccaatcgtggcgtgtcggcctctcttcggagaggcactggcgccggagaggcactggcgccggggcc

SEQ ID NO: 20 - RNA, 101 nt, synthetic construct
  misc_feature 1..101: note = Synthetic sequence
  source 1..101: mol_type = other RNA
gcccggatagctcagtcggtagagcagcggccgcgaggaaggaggtctgaggaggtcactgcggccgcgggtccagggttcaagtccctgttcgggcgcca

SEQ ID NO: 21 - RNA, 65 nt, synthetic construct
  misc_feature 1..65: note = Synthetic sequence
  source 1..65: mol_type = other RNA
tccccaatcgtggcgtgtcggcggaagaaactgtggcacttcggtgccaggcactggcgccggga

SEQ ID NO: 22 - RNA, 57 nt, synthetic construct
  misc_feature 1..57: note = Synthetic sequence
  source 1..57: mol_type = other RNA
tccccaatcgtggcgtgtcgtaagaagagcacgtatacgcaataactggcgccggga

SEQ ID NO: 23 - RNA, 196 nt, synthetic construct
  misc_feature 1..196: note = Synthetic sequence
  source 1..196: mol_type = other RNA
cccacggaggatccccaatcgtggcgtgtcggcctctcccaatcgtggcgtgtcggcctctcccaatcgtggcgtgtcggcctctcccaatcgtggcgtgtcggcctctcttcggagaggcactggcgccggagaggcactggcgccggagaggcactggcgccggagaggcactggcgccgggatcctccgtggg

SEQ ID NO: 24 - RNA, 1227 nt, synthetic construct
  misc_feature 1..1227: note = Synthetic sequence
  source 1..1227: mol_type = other RNA
atggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaactcgctagagcatggttaagcttcccacggaggatccccaatcgtggcgtgtcggcctctcccaatcgtggcgtgtcggcctctcccaatcgtggcgtgtcggcctctcccaatcgtggcgtgtcggcctctcttcggagaggcactggcgccggagaggcactggcgccggagaggcactggcgccggagaggcactggcgccgggatcctccgtggg

SEQ ID NO: 25 - RNA, 595 nt, synthetic construct
  misc_feature 1..595: note = Synthetic sequence
  source 1..595: mol_type = other RNA
tgggaaatacacatttgctgctcacatggatggaacatacaaattttgttttagtaaccggatgtccaccatgactccaaaaatagtgatgttcaccattgatattggggaggctccaaaaggacaagatatggaaacagaagctcaccagaacaagctagaagaaatgatcaatgagctagcagtggcgatgacagctgtaaagcacgaacaggaatacatggaagtccgggagagaatacacagagccatcaacgacaacacaaacagcagagtggtcctttggtccttctttgaagctcttgttctagttgccatgacattgggacagatctactacctgaagagattttttgaagtccggagagttgtttaactcgctagagcatggttaagcttcccacggaggatccccaatcgtggcgtgtcggcctctcccaatcgtggcgtgtcggcctctcccaatcgtggcgtgtcggcctctcccaatcgtggcgtgtcggcctctcttcggagaggcactggcgccggagaggcactggcgccggagaggcactggcgccggagaggcactggcgccgggatcctccgtggg

SEQ ID NO: 26 - RNA, 930 nt, synthetic construct
  misc_feature 1..930: note = Synthetic sequence
  source 1..930: mol_type = other RNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgctagagcatggttaagcttcccacggaggatccccaatcgtggcgtgtcggcctctcccaatcgtggcgtgtcggcctctcccaatcgtggcgtgtcggcctctcccaatcgtggcgtgtcggcctctcttcggagaggcactggcgccggagaggcactggcgccggagaggcactggcgccggagaggcactggcgccgggatcctccgtggg

SEQ ID NO: 27 - RNA, 935 nt, synthetic construct
  misc_feature 1..935: note = Synthetic sequence
  source 1..935: mol_type = other RNA
atgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagctcaattaacactcgctagagcatggttggtaccgtagtcaagcttcccacggaggatccccaatcgtggcgtgtcggcctctcccaatcgtggcgtgtcggcctctcccaatcgtggcgtgtcggcctctcccaatcgtggcgtgtcggcctctcttcggagaggcactggcgccggagaggcactggcgccggagaggcactggcgccggagaggcactggcgccgggatcctccgtggg

SEQ ID NO: 28 - RNA, 142 nt, synthetic construct
  misc_feature 1..142: note = Synthetic sequence
  source 1..142: mol_type = other RNA
gaatctgcaagtggatattgtttgagagctaggccccaatcgtggcgtgtcggcctgcttcggcaggcactggcgccgggcctagcaagttcaaataaggctagtccgttatcaacttgaaaaagtggcaccgagtcggtgc

SEQ ID NO: 29 - RNA, 142 nt, synthetic construct
  misc_feature 1..142: note = Synthetic sequence
  source 1..142: mol_type = other RNA
gaatctgcaagtggatattgtttgagagctagaaatagcaagttcaaataaggctagtccgttctcaacttggccccaatcgtggcgtgtcggcctgcttcggcaggcactggcgccgggccaagtggcaccgagtcggtgc

SEQ ID NO: 30 - RNA, 189 nt, synthetic construct
  misc_feature 1..189: note = Synthetic sequence
  source 1..189: mol_type = other RNA
gaatctgcaagtggatattgtttgagagctaggccccaatcgtggcgtgtcggcctgcttcggcaggcactggcgccgggcctagcaagttcaaataaggctagtccgttctcaacttggccccaatcgtggcgtgtcggcctgcttcggcaggcactggcgccgggccaagtggcaccgagtcggtgc

SEQ ID NO: 31 - RNA, 214 nt, synthetic construct
  misc_feature 1..214: note = Synthetic sequence
  source 1..214: mol_type = other RNA
ggggccccccaatcgtggcgtgtcggcctctcccaatcgtggcgtgtcggcctctcccaatcgtggcgtgtcggcctctcccaatcgtggcgtgtcggcctctcggccaacatgaggatcacccatgtctgcagggccgagaggcactggcgccggagaggcactggcgccggagaggcactggcgccggagaggcactggcgccgggggcccc

SEQ ID NO: 32 - DNA, 51 bp, synthetic construct
  misc_feature 1..51: note = Synthetic sequence
  source 1..51: mol_type = other DNA
gatcccgcgaaattaatacgactcactatagggttgccatgtgtatgtggg

SEQ ID NO: 33 - DNA, 31 bp, synthetic construct
  misc_feature 1..31: note = Synthetic sequence
  source 1..31: mol_type = other DNA
caaggggttatgctattgccatgaatgatcc

SEQ ID NO: 34 - DNA, 37 bp, synthetic construct
  misc_feature 1..37: note = Synthetic sequence
  source 1..37: mol_type = other DNA
tagcataaccccttggggcctctaaacgggtcttgag

SEQ ID NO: 35 - DNA, 39 bp, synthetic construct
  misc_feature 1..39: note = Synthetic sequence
  source 1..39: mol_type = other DNA
atttcgcgggatcgagatctcgatcctctacgccggacg

SEQ ID NO: 36 - DNA, 38 bp, synthetic construct
  misc_feature 1..38: note = Synthetic sequence
  source 1..38: mol_type = other DNA
ggaatattaagctcgcccttttgccatgtgtatgtggg

SEQ ID NO: 37 - DNA, 37 bp, synthetic construct
  misc_feature 1..37: note = Synthetic sequence
  source 1..37: mol_type = other DNA
tgacctcgaagctcgcccttgttgccatgaatgatcc

SEQ ID NO: 38 - DNA, 38 bp, synthetic construct
  misc_feature 1..38: note = Synthetic sequence
  source 1..38: mol_type = other DNA
ggaaaggacgaaactctagattgccatgtgtatgtggg

SEQ ID NO: 39 - DNA, 44 bp, synthetic construct
  misc_feature 1..44: note = Synthetic sequence
  source 1..44: mol_type = other DNA
tgtctcgaggtcgagaattcaaaaaaagttgccatgaatgatcc

SEQ ID NO: 40 - DNA, 39 bp, synthetic construct
  misc_feature 1..39: note = Synthetic sequence
  source 1..39: mol_type = other DNA
ggaaaggacgaaactctagagcccggatagctcagtcgg

SEQ ID NO: 41 - DNA, 78 bp, synthetic construct
  misc_feature 1..78: note = Synthetic sequenc
  source 1..78: mol_type = other DNA
tgtctcgaggtcgagaattcaaaaaaatggcgcccgaacagggacttgcgagctcaggatccttccgtttcgcactgg

SEQ ID NO: 42 - DNA, 40 bp, synthetic construct
  misc_feature 1..40: note = Synthetic sequence
  source 1..40: mol_type = other DNA
tagcgtttaaacttaagcttcccacggaggatccccaatc

SEQ ID NO: 43 - DNA, 41 bp, synthetic construct
  misc_feature 1..41: note = Synthetic sequence
  source 1..41: mol_type = other DNA
acgggccctctagactcgagcccacggaggatcccggcgcc

SEQ ID NO: 44 - DNA, 40 bp, synthetic construct
  misc_feature 1..40: note = Synthetic sequence
  source 1..40: mol_type = other DNA
ggagacccaagctggctagcatggggaaggtgaaggtcgg

SEQ ID NO: 45 - DNA, 59 bp, synthetic construct
  misc_feature 1..59: note = Synthetic sequence
  source 1..59: mol_type = other DNA
ggatcctccgtgggaagcttaaccatgctctagcgagtgttactccttggaggccatgt

SEQ ID NO: 46 - DNA, 40 bp, synthetic construct
  misc_feature 1..40: note = Synthetic sequence
  source 1..40: mol_type = other DNA
ggagacccaagctggctagcatggtgacgcttgctgaact

SEQ ID NO: 47 - DNA, 58 bp, synthetic construct
  misc_feature 1..58: note = Synthetic sequence
  source 1..58: mol_type = other DNA
ggatcctccgtgggaagcttaaccatgctctagcgagttaaacaactctccggacttc

SEQ ID NO: 48 - DNA, 42 bp, synthetic construct
  misc_feature 1..42: note = Synthetic sequence
  source 1..42: mol_type = other DNA
ggagacccaagctggctagcatggtgagcaagggcgaggagg

SEQ ID NO: 49 - DNA, 58 bp, synthetic construct
  misc_feature 1..58: note = Synthetic sequence
  source 1..58: mol_type = other DNA
ggatcctccgtgggaagctaaccatgctctagcgagttacttgtacagctcgtccatg

SEQ ID NO: 50 - DNA, 40 bp, synthetic construct
  misc_feature 1..40: note = Synthetic sequence
  source 1..40: mol_type = other DNA
ggagacccaagctggctagcatgagcgagctgattaagga

SEQ ID NO: 51 - DNA, 65 bp, synthetic construct
  misc_feature 1..65: note = Synthetic sequence
  source 1..65: mol_type = other DNA
ggatcctccgtgggaagcttctcccaaaccatgctctagcgagtgttaattgagcttgtgcccca

SEQ ID NO: 52 - DNA, 41 bp, synthetic construct
  misc_feature 1..41: note = Synthetic sequence
  source 1..41: mol_type = other DNA
aaaggacgaaacaccgaatctgcaagtggatattgtttgag

SEQ ID NO: 53 - DNA, 33 bp, synthetic construct
  misc_feature 1..33: note = Synthetic sequence
  source 1..33: mol_type = other DNA
tgatctagaaaaaaagcaccgactcggtgccac

SEQ ID NO: 54 - DNA, 29 bp, synthetic construct
  misc_feature 1..29: note = Synthetic sequence
  source 1..29: mol_type = other DNA
tttttttctagatcataatcagccatacc

SEQ ID NO: 55 - DNA, 22 bp, synthetic construct
  misc_feature 1..22: note = Synthetic sequence
  source 1..22: mol_type = other DNA
ggtgtttcgtcctttccacaag

SEQ ID NO: 56 - DNA, 46 bp, synthetic construct
  misc_feature 1..46: note = Synthetic sequence
  source 1..46: mol_type = other DNA
tagcgtttaaacttaagcttgtgcaggctggcgccaccatggcccc

SEQ ID NO: 57 - DNA, 43 bp, synthetic construct
  misc_feature 1..43: note = Synthetic sequence
  source 1..43: mol_type = other DNA
acgggccctctagactcgagttacttgtacagctcgtccatgc

SEQ ID NO: 58 - DNA, 38 bp, synthetic construct
  misc_feature 1..38: note = Synthetic sequence
  source 1..38: mol_type = other DNA
ggaaaggacgaaactctagaggggccccccaatcgtgg

SEQ ID NO: 59 - DNA, 46 bp, synthetic construct
  misc_feature 1..46: note = Synthetic sequence
  source 1..46: mol_type = other DNA
tgtctcgaggtcgagaattcaaaaaaaggggcccccggcgccagtg

SEQ ID NO: 60 - DNA, 49 bp, synthetic construct
  misc_feature 1..49: note = Synthetic sequence
  source 1..49: mol_type = other DNA
gaaccgtcagatccgctagccaccatgggctacccctacgacgtgcccg

SEQ ID NO: 61 - DNA, 23 bp, synthetic construct
  misc_feature 1..23: note = Synthetic sequence
  source 1..23: mol_type = other DNA
tccagaatccgcgtagatgccgg

SEQ ID NO: 62 - DNA, 49 bp, synthetic construct
  misc_feature 1..49: note = Synthetic sequence
  source 1..49: mol_type = other DNA
ctacgcggattctggaggcggtggatccatgagcgagctgattaaggag

SEQ ID NO: 63 - DNA, 28 bp, synthetic construct
  misc_feature 1..28: note = Synthetic sequence
  source 1..28: mol_type = other DNA
agatctattgagcttgtgccccagtttg

SEQ ID NO: 64 - DNA, 37 bp, synthetic construct
  misc_feature 1..37: note = Synthetic sequence
  source 1..37: mol_type = other DNA
caagctcaatagatctatgcctgaaccggcaaaatcc

SEQ ID NO: 65 - DNA, 40 bp, synthetic construct
  misc_feature 1..40: note = Synthetic sequence
  source 1..40: mol_type = other DNA
gactgcagaattcgaagcttacttggagctggtgtacttg

SEQ ID NO: 66 - DNA, 53 bp, synthetic construct
  misc_feature 1..53: note = Combined DNA/RNA molecule
  source 1..53: mol_type = other DNA
ggcccccaatcgtggcgtgtcggcctgcttcggcaggcactggggccggggcc

SEQ ID NO: 67 - RNA, 53 nt, synthetic construct
  misc_feature 1..53: note = Synthetic sequence Figure 5B
  source 1..53: mol_type = other RNA
ggcccccaatcgtggcgtgtcggcctgcttcggcaggcactggggccggggcc

SEQ ID NO: 68 - RNA, 49 nt, synthetic construct
  misc_feature 1..49: note = Synthetic sequence Figure 10(1)
  source 1..49: mol_type = other RNA
tccccaatcgtggcgtgtcggcctgcttcggcaggcactggggccggga

SEQ ID NO: 69 - RNA, 28 nt, synthetic construct
  misc_feature 1..28: note = Synthetic sequence Figure 10(2)
  source 1..28: mol_type = other RNA
ggaagaaactgtggcacttcggtgccag

SEQ ID NO: 70 - RNA, 20 nt, synthetic construct
  misc_feature 1..20: note = Synthetic sequence Figure 10(3)
  source 1..20: mol_type = other RNA
agaagagcacgtatacgcaa

SEQ ID NO: 71 - RNA, 32 nt, synthetic construct
  misc_feature 1..32: note = Synthetic sequence
  misc_feature join(1,19,20,21,32): note = n are nucleotide fragments greater than or equal to 1 in length
  source 1..32: mol_type = other RNA
nccaatcgtggcgtgtcgnnnactggcgccgn

SEQ ID NO: 72 - RNA, 32 nt, synthetic construct
  misc_feature 1..32: note = Synthetic sequence
  misc_feature join(1,19,20,21,32): note = n are nucleotide fragments greater than or equal to 1 in length
  source 1..32: mol_type = other RNA
ncaaatcgtggcgtgtcgnnnactggcgccgn

SEQ ID NO: 73 - RNA, 32 nt, synthetic construct
  misc_feature 1..32: note = Synthetic sequence
  misc_feature join(1,19,20,21,32): note = n are nucleotide fragments greater than or equal to 1 in length
  source 1..32: mol_type = other RNA
nctaatcgtggcgtgtcgnnnactggcgccgn

SEQ ID NO: 74 - DNA, 32 bp, synthetic construct
  misc_feature 1..32: note = Synthetic sequence
  misc_feature 32: note = r encodes n, n are nucleotide fragments greater than or equal to 1 in length
  source 1..32: mol_type = other DNA
rccaatcgtggcgtgtcgrrractggcgccgn

SEQ ID NO: 75 - RNA, 52 nt, synthetic construct
  misc_feature 1..52: note = F30 scaffold RNA
  misc_structure 1..52
  source 1..52: mol_type = other RNA
ttgccatgtgtatgtgggttcgcccacatactctgatgatcattcatggcaa

SEQ ID NO: 76 - RNA, 65 nt, synthetic construct
  misc_feature 1..65: note = tRNA scaffold RNA
  tRNA 1..65
  source 1..65: mol_type = other RNA
gcccggatagctcagtcggtagagcagcgcgcgggtccagggttcaagtccctgttcgggcgcca

SEQ ID NO: 77 - RNA, 49 nt, synthetic construct
  misc_feature 1..49: note = Pepper-2
  source 1..49: mol_type = other RNA
ctgccaatcgtggcgtgtcggcctgcttcggcaggcactggccaggccg

SEQ ID NO: 78 - RNA, 32 nt, synthetic construct
  misc_feature 1..32: note = Pepper (A4U)
  misc_feature join(1,19,20,21,32): note = n are nucleotide fragments greater than or equal to 1 in length
  source 1..32: mol_type = other RNA
ncctatcgtggcgtgtcgnnnactggcgccgn

SEQ ID NO: 79 - RNA, 32 nt, synthetic construct
  misc_feature 1..32: note = Pepper (A4G)
  misc_feature join(1,19,20,21,32): note = n are nucleotide fragments greater than or equal to 1 in length
  source 1..32: mol_type = other RNA
nccgatcgtggcgtgtcgnnnactggcgccgn

SEQ ID NO: 80 - RNA, 32 nt, synthetic construct
  misc_feature 1..32: note = Pepper (A4C)
  misc_feature join(1,19,20,21,32): note = n are nucleotide fragments greater than or equal to 1 in length
  source 1..32: mol_type = other RNA
ncccatcgtggcgtgtcgnnnactggcgccgn

SEQ ID NO: 81 - RNA, 32 nt, synthetic construct
  misc_feature 1..32: note = Pepper (A5G)
  misc_feature join(1,19,20,21,32): note = n are nucleotide fragments greater than or equal to 1 in length
  source 1..32: mol_type = other RNA
nccagtcgtggcgtgtcgnnnactggcgccgn

SEQ ID NO: 82 - RNA, 32 nt, synthetic construct
  misc_feature 1..32: note = Pepper (A5C)
  misc_feature join(1,19,20,21,32): note = n are nucleotide fragments greater than or equal to 1 in length
  source 1..32: mol_type = other RNA
nccactcgtggcgtgtcgnnnactggcgccgn

SEQ ID NO: 83 - RNA, 32 nt, synthetic construct
  misc_feature 1..32: note = Pepper (U6A)
  misc_feature join(1,19,20,21,32): note = n are nucleotide fragments greater than or equal to 1 in length
  source 1..32: mol_type = other RNA
nccaaacgtggcgtgtcgnnnactggcgccgn

SEQ ID NO: 84 - RNA, 32 nt, synthetic construct
  misc_feature 1..32: note = Pepper (U6G)
  misc_feature join(1,19,20,21,32): note = n are nucleotide fragments greater than or equal to 1 in length
  source 1..32: mol_type = other RNA
nccaagcgtggcgtgtcgnnnactggcgccgn

SEQ ID NO: 85 - RNA, 32 nt, synthetic construct
  misc_feature 1..32: note = Pepper (U6C)
  misc_feature join(1,19,20,21,32): note = n are nucleotide fragments greater than or equal to 1 in length
  source 1..32: mol_type = other RNA
nccaaccgtggcgtgtcgnnnactggcgccgn

SEQ ID NO: 86 - RNA, 32 nt, synthetic construct
  misc_feature 1..32: note = Pepper (C7A)
  misc_feature join(1,19,20,21,32): note = n are nucleotide fragments greater than or equal to 1 in length
  source 1..32: mol_type = other RNA
nccaatagtggcgtgtcgnnnactggcgccgn

SEQ ID NO: 87 - RNA, 32 nt, synthetic construct
  misc_feature 1..32: note = Pepper (C7U)
  misc_feature join(1,19,20,21,32): note = n are nucleotide fragments greater than or equal to 1 in length
  source 1..32: mol_type = other RNA
nccaattgtggcgtgtcgnnnactggcgccgn

SEQ ID NO: 88 - RNA, 32 nt, synthetic construct
  misc_feature 1..32: note = Pepper (G8C)
  misc_feature join(1,19,20,21,32): note = n are nucleotide fragments greater than or equal to 1 in length
  source 1..32: mol_type = other RNA
nccaatcctggcgtgtcgnnnactggcgccgn

SEQ ID NO: 89 - RNA, 32 nt, synthetic construct
  misc_feature 1..32: note = Pepper (U9A)
  misc_feature join(1,19,20,21,32): note = n are nucleotide fragments greater than or equal to 1 in length
  source 1..32: mol_type = other RNA
nccaatcgaggcgtgtcgnnnactggcgccgn

SEQ ID NO: 90 - RNA, 32 nt, synthetic construct
  misc_feature 1..32: note = Pepper (G11A)
  misc_feature join(1,19,20,21,32): note = n are nucleotide fragments greater than or equal to 1 in length
  source 1..32: mol_type = other RNA
nccaatcgtgacgtgtcgnnnactggcgccgn

SEQ ID NO: 91 - RNA, 32 nt, synthetic construct
  misc_feature 1..32: note = Pepper (G11U)
  misc_feature join(1,19,20,21,32): note = n are nucleotide fragments greater than or equal to 1 in length
  source 1..32: mol_type = other RNA
nccaatcgtgtcgtgtcgnnnactggcgccgn

SEQ ID NO: 92 - RNA, 32 nt, synthetic construct
  misc_feature 1..32: note = Pepper (C12G)
  misc_feature join(1,19,20,21,32): note = n are nucleotide fragments greater than or equal to 1 in length
  source 1..32: mol_type = other RNA
nccaatcgtggggtgtcgnnnactggcgccgn

SEQ ID NO: 93 - RNA, 32 nt, synthetic construct
  misc_feature 1..32: note = Pepper (C12A)
  misc_feature join(1,19,20,21,32): note = n are nucleotide fragments greater than or equal to 1 in length
  source 1..32: mol_type = other RNA
nccaatcgtggagtgtcgnnnactggcgccgn

SEQ ID NO: 94 - RNA, 32 nt, synthetic construct
  misc_feature 1..32: note = Pepper (C12U)
  misc_feature join(1,19,20,21,32): note = n are nucleotide fragments greater than or equal to 1 in length
  source 1..32: mol_type = other RNA
nccaatcgtggtgtgtcgnnnactggcgccgn

SEQ ID NO: 95 - RNA, 32 nt, synthetic construct
  misc_feature 1..32: note = Pepper (G13C)
  misc_feature join(1,19,20,21,32): note = n are nucleotide fragments greater than or equal to 1 in length
  source 1..32: mol_type = other RNA
nccaatcgtggcctgtcgnnnactggcgccgn

SEQ ID NO: 96 - RNA, 32 nt, synthetic construct
  misc_feature 1..32: note = Pepper (U14A)
  misc_feature join(1,19,20,21,32): note = n are nucleotide fragments greater than or equal to 1 in length
  source 1..32: mol_type = other RNA
nccaatcgtggcgagtcgnnnactggcgccgn

SEQ ID NO: 97 - RNA, 32 nt, synthetic construct
  misc_feature 1..32: note = Pepper (U14G)
  misc_feature join(1,19,20,21,32): note = n are nucleotide fragments greater than or equal to 1 in length
  source 1..32: mol_type = other RNA
nccaatcgtggcgggtcgnnnactggcgccgn

SEQ ID NO: 98 - RNA, 32 nt, synthetic construct
  misc_feature 1..32: note = Pepper (C17U)
  misc_feature join(1,19,20,21,32): note = n are nucleotide fragments greater than or equal to 1 in length
  source 1..32: mol_type = other RNA
nccaatcgtggcgtgttgnnnactggcgccgn

SEQ ID NO: 99 - RNA, 32 nt, synthetic construct
  misc_feature 1..32: note = Pepper (G18U)
  misc_feature join(1,19,20,21,32): note = n are nucleotide fragments greater than or equal to 1 in length
  source 1..32: mol_type = other RNA
nccaatcgtggcgtgtctnnnactggcgccgn

SEQ ID NO: 100 - RNA, 32 nt, synthetic construct
  misc_feature 1..32: note = Pepper (G18C)
  misc_feature join(1,19,20,21,32): note = n are nucleotide fragments greater than or equal to 1 in length
  source 1..32: mol_type = other RNA
nccaatcgtggcgtgtccnnnactggcgccgn

SEQ ID NO: 101 - RNA, 32 nt, synthetic construct
  misc_feature 1..32: note = Pepper (C27G)
  misc_feature join(1,19,20,21,32): note = n are nucleotide fragments greater than or equal to 1 in length
  source 1..32: mol_type = other RNA
nccaatcgtggcgtgtcgnnnactggggccgn

SEQ ID NO: 102 - RNA, 32 nt, synthetic construct
  misc_feature 1..32: note = Pepper (C27U)
  misc_feature join(1,19,20,21,32): note = n are nucleotide fragments greater than or equal to 1 in length
  source 1..32: mol_type = other RNA
nccaatcgtggcgtgtcgnnnactggtgccgn

SEQ ID NO: 103 - RNA, 32 nt, synthetic construct
  misc_feature 1..32: note = Pepper (G28U)
  misc_feature join(1,19,20,21,32): note = n are nucleotide fragments greater than or equal to 1 in length
  source 1..32: mol_type = other RNA
nccaatcgtggcgtgtcgnnnactggctccgn

SEQ ID NO: 104 - RNA, 32 nt, synthetic construct
  misc_feature 1..32: note = Pepper (C29G)
  misc_feature join(1,19,20,21,32): note = n are nucleotide fragments greater than or equal to 1 in length
  source 1..32: mol_type = other RNA
nccaatcgtggcgtgtcgnnnactggcggcgn

SEQ ID NO: 105 - RNA, 32 nt, synthetic construct
  misc_feature 1..32: note = Pepper (C29U)
  misc_feature join(1,19,20,21,32): note = n are nucleotide fragments greater than or equal to 1 in length
  source 1..32: mol_type = other RNA
nccaatcgtggcgtgtcgnnnactggcgtcgn

SEQ ID NO: 106 - RNA, 32 nt, synthetic construct
  misc_feature 1..32: note = Pepper (C30A)
  misc_feature join(1,19,20,21,32): note = n are nucleotide fragments greater than or equal to 1 in length
  source 1..32: mol_type = other RNA
nccaatcgtggcgtgtcgnnnactggcgcagn

SEQ ID NO: 107 - RNA, 32 nt, synthetic construct
  misc_feature 1..32: note = Pepper (C30U)
  misc_feature join(1,19,20,21,32): note = n are nucleotide fragments greater than or equal to 1 in length
  source 1..32: mol_type = other RNA
nccaatcgtggcgtgtcgnnnactggcgctgn

SEQ ID NO: 108 - RNA, 32 nt, synthetic construct
  misc_feature 1..32: note = Pepper (C2G/G31C)
  misc_feature join(1,19,20,21,32): note = n are nucleotide fragments greater than or equal to 1 in length
  source 1..32: mol_type = other RNA
ngcaatcgtggcgtgtcgnnnactggcgcccn

SEQ ID NO: 109 - RNA, 32 nt, synthetic construct
  misc_feature 1..32: note = Pepper (C2U/G31A)
  misc_feature join(1,19,20,21,32): note = n are nucleotide fragments greater than or equal to 1 in length
  source 1..32: mol_type = other RNA
ntcaatcgtggcgtgtcgnnnactggcgccan

SEQ ID NO: 110 - RNA, 32 nt, synthetic construct
  misc_feature 1..32: note = Pepper (C2A/G31U)
  misc_feature join(1,19,20,21,32): note = n are nucleotide fragments greater than or equal to 1 in length
  source 1..32: mol_type = other RNA
nacaatcgtggcgtgtcgnnnactggcgcctn

SEQ ID NO: 111 - RNA, 32 nt, synthetic construct
  misc_feature 1..32: note = Pepper (C10A/C30U)
  misc_feature join(1,19,20,21,32): note = n are nucleotide fragments greater than or equal to 1 in length
  source 1..32: mol_type = other RNA
nccaatcgtagcgtgtcgnnnactggcgctgn

SEQ ID NO: 112 - RNA, 32 nt, synthetic construct
  misc_feature 1..32: note = Pepper (C10C/C30G)
  misc_feature join(1,19,20,21,32): note = n are nucleotide fragments greater than or equal to 1 in length
  source 1..32: mol_type = other RNA
nccaatcgtcgcgtgtcgnnnactggcgcggn

SEQ ID NO: 113 - RNA, 32 nt, synthetic construct
  misc_feature 1..32: note = Pepper (C10U/C30A)
  misc_feature join(1,19,20,21,32): note = n are nucleotide fragments greater than or equal to 1 in length
  source 1..32: mol_type = other RNA
nccaatcgttgcgtgtcgnnnactggcgcagn

SEQ ID NO: 114 - RNA, 32 nt, synthetic construct
  misc_feature 1..32: note = Pepper (C2G/G31/C3A)
  misc_feature join(1,19,20,21,32): note = n are nucleotide fragments greater than or equal to 1 in length
  source 1..32: mol_type = other RNA
ngaaatcgtggcgtgtcgnnnactggcgcccn

SEQ ID NO: 115 - RNA, 32 nt, synthetic construct
  misc_feature 1..32: note = Pepper (C2G/G31C/A4C)
  misc_feature join(1,19,20,21,32): note = n are nucleotide fragments greater than or equal to 1 in length
  source 1..32: mol_type = other RNA
ngccatcgtggcgtgtcgnnnactggcgcccn

SEQ ID NO: 116 - RNA, 32 nt, synthetic construct
  misc_feature 1..32: note = Pepper (C2G/G31C/A5C)
  misc_feature join(1,19,20,21,32): note = n are nucleotide fragments greater than or equal to 1 in length
  source 1..32: mol_type = other RNA
ngcactcgtggcgtgtcgnnnactggcgcccn

SEQ ID NO: 117 - RNA, 32 nt, synthetic construct
  misc_feature 1..32: note = Pepper (C2G/G31C/G8C)
  misc_feature join(1,19,20,21,32): note = n are nucleotide fragments greater than or equal to 1 in length
  source 1..32: mol_type = other RNA
ngcaatcctggcgtgtcgnnnactggcgcccn

SEQ ID NO: 118 - RNA, 32 nt, synthetic construct
  misc_feature 1..32: note = Pepper (C2G/G31C/C12U)
  misc_feature join(1,19,20,21,32): note = n are nucleotide fragments greater than or equal to 1 in length
  source 1..32: mol_type = other RNA
ngcaatcgtggtgtgtcgnnnactggcgcccn

SEQ ID NO: 119 - RNA, 32 nt, synthetic construct
  misc_feature 1..32: note = Pepper (C2G/G31C/U14G)
  misc_feature join(1,19,20,21,32): note = n are nucleotide fragments greater than or equal to 1 in length
  source 1..32: mol_type = other RNA
ngcaatcgtggcgggtcgnnnactggcgcccn

SEQ ID NO: 120 - RNA, 32 nt, synthetic construct
  misc_feature 1..32: note = Pepper (C2G/G31C/C27U)
  misc_feature join(1,19,20,21,32): note = n are nucleotide fragments greater than or equal to 1 in length
  source 1..32: mol_type = other RNA
ngcaatcgtggcgtgtcgnnnactggtgcccn

SEQ ID NO: 121 - RNA, 32 nt, synthetic construct
  misc_feature 1..32: note = Pepper (C2G/G31C/C29G)
  misc_feature join(1,19,20,21,32): note = n are nucleotide fragments greater than or equal to 1 in length
  source 1..32: mol_type = other RNA
ngcaatcgtggcgtgtcgnnnactggcggccn

SEQ ID NO: 122 - RNA, 32 nt, synthetic construct
  misc_feature 1..32: note = Pepper (C2G/G31C/C30U)
  misc_feature join(1,19,20,21,32): note = n are nucleotide fragments greater than or equal to 1 in length
  source 1..32: mol_type = other RNA
ngcaatcgtggcgtgtcgnnnactggcgctcn

SEQ ID NO: 123 - RNA, 32 nt, synthetic construct
  misc_feature 1..32: note = Pepper (C2G/G31C/G10A/C30U)
  misc_feature join(1,19,20,21,32): note = n are nucleotide fragments greater than or equal to 1 in length
  source 1..32: mol_type = other RNA
ngcaatcgtagcgtgtcgnnnactggcgctcn

SEQ ID NO: 124 - RNA, 32 nt, synthetic construct
  misc_feature 1..32: note = Pepper (C2G/G31C/G10C/C30G)
  misc_feature join(1,19,20,21,32): note = n are nucleotide fragments greater than or equal to 1 in length
  source 1..32: mol_type = other RNA
ngcaatcgtcgcgtgtcgnnnactggcgcgcn

SEQ ID NO: 125 - RNA, 32 nt, synthetic construct
  misc_feature 1..32: note = Pepper (C2G/G31C/G10U/C30A)
  misc_feature join(1,19,20,21,32): note = n are nucleotide fragments greater than or equal to 1 in length
  source 1..32: mol_type = other RNA
ngcaatcgttgcgtgtcgnnnactggcgcacn

SEQ ID NO: 126 - RNA, 32 nt, synthetic construct
  misc_feature 1..32: note = Pepper (C2U/G31A/G10C/C30G)
  misc_feature join(1,19,20,21,32): note = n are nucleotide fragments greater than or equal to 1 in length
  source 1..32: mol_type = other RNA
ntcaatcgtcgcgtgtcgnnnactggcgcgan

SEQ ID NO: 127 - RNA, 32 nt, synthetic construct
  misc_feature 1..32: note = Pepper (C2U/G31A/G10U/C30A)
  misc_feature join(1,19,20,21,32): note = n are nucleotide fragments greater than or equal to 1 in length
  source 1..32: mol_type = other RNA
ntcaatcgttgcgtgtcgnnnactggcgcaan

SEQ ID NO: 128 - RNA, 32 nt, synthetic construct
  misc_feature 1..32: note = Pepper (C2A/G31U/G10A/C30U)
  misc_feature join(1,19,20,21,32): note = n are nucleotide fragments greater than or equal to 1 in length
  source 1..32: mol_type = other RNA
nacaatcgtagcgtgtcgnnnactggcgcttn

SEQ ID NO: 129 - RNA, 32 nt, synthetic construct
  misc_feature 1..32: note = Pepper (C2A/G31U/G10C/C30G)
  misc_feature join(1,19,20,21,32): note = n are nucleotide fragments greater than or equal to 1 in length
  source 1..32: mol_type = other RNA
nacaatcgtcgcgtgtcgnnnactggcgcgtn

SEQ ID NO: 130 - RNA, 32 nt, synthetic construct
  misc_feature 1..32: note = Pepper (C2A/G31U/G10U/C30A)
  misc_feature join(1,19,20,21,32): note = n are nucleotide fragments greater than or equal to 1 in length
  source 1..32: mol_type = other RNA
nacaatcgttgcgtgtcgnnnactggcgcatn

SEQ ID NO: 131 - RNA, 32 nt, synthetic construct
  misc_feature 1..32: note = Pepper (C2G/G31C/G10C/C30G/C3A)
  misc_feature join(1,19,20,21,32): note = n are nucleotide fragments greater than or equal to 1 in length
  source 1..32: mol_type = other RNA
ngaaatcgtcgcgtgtcgnnnactggcgcgcn

SEQ ID NO: 132 - RNA, 32 nt, synthetic construct
  misc_feature 1..32: note = Pepper (C2G/G31C/G10C/C30G/A4C)
  misc_feature join(1,19,20,21,32): note = n are nucleotide fragments greater than or equal to 1 in length
  source 1..32: mol_type = other RNA
ngccatcgtcgcgtgtcgnnnactggcgcgcn

SEQ ID NO: 133 - RNA, 32 nt, synthetic construct
  misc_feature 1..32: note = Pepper (C2G/G31C/G10C/C30G/A5C)
  misc_feature join(1,19,20,21,32): note = n are nucleotide fragments greater than or equal to 1 in length
  source 1..32: mol_type = other RNA
ngcactcgtcgcgtgtcgnnnactggcgcgcn

SEQ ID NO: 134 - RNA, 32 nt, synthetic construct
  misc_feature 1..32: note = Pepper (C2G/G31C/G10C/C30G/G8C)
  misc_feature join(1,19,20,21,32): note = n are nucleotide fragments greater than or equal to 1 in length
  source 1..32: mol_type = other RNA
ngcaatcctcgcgtgtcgnnnactggcgcgcn

SEQ ID NO: 135 - RNA, 32 nt, synthetic construct
  misc_feature 1..32: note = Pepper (C2G/G31C/G10C/C30G/C12U)
  misc_feature join(1,19,20,21,32): note = n are nucleotide fragments greater than or equal to 1 in length
  source 1..32: mol_type = other RNA
ngcaatcgtcgtgtgtcgnnnactggcgcgcn

SEQ ID NO: 136 - RNA, 32 nt, synthetic construct
  misc_feature 1..32: note = Pepper (C2G/G31C/G10C/C30G/U14G)
  misc_feature join(1,19,20,21,32): note = n are nucleotide fragments greater than or equal to 1 in length
  source 1..32: mol_type = other RNA
ngcaatcgtcgcgggtcgnnnactggcgcgcn

SEQ ID NO: 137 - RNA, 32 nt, synthetic construct
  misc_feature 1..32: note = Pepper (C2G/G31C/G10C/C30G/C27U)
  misc_feature join(1,19,20,21,32): note = n are nucleotide fragments greater than or equal to 1 in length
  source 1..32: mol_type = other RNA
ngcaatcgtcgcgggtcgnnnactggtgcgcn

SEQ ID NO: 138 - RNA, 32 nt, synthetic construct
  misc_feature 1..32: note = Pepper (C2G/G31C/G10C/C30G/C29G)
  misc_feature join(1,19,20,21,32): note = n are nucleotide fragments greater than or equal to 1 in length
  source 1..32: mol_type = other RNA
ngcaatcgtcgcgggtcgnnnactggcgggcn

SEQ ID NO: 139 - RNA, 32 nt, synthetic construct; identical to SEQ ID NO: 138
  misc_feature 1..32: note = Pepper (C2G/G31C/G10C/C30G/C29G)
  misc_feature join(1,19,20,21,32): note = n are nucleotide fragments greater than or equal to 1 in length
  source 1..32: mol_type = other RNA

SEQ ID NO: 140 - RNA, 32 nt, synthetic construct
  misc_feature 1..32: note = Pepper (C2G/G31C/G10A/C30U/U6G/C27U)
  misc_feature join(1,19,20,21,32): note = n are nucleotide fragments greater than or equal to 1 in length
  source 1..32: mol_type = other RNA
ngcaagcgtagcgtgtcgnnnactggtgctcn

SEQ ID NO: 141 - RNA, 32 nt, synthetic construct
  misc_feature 1..32: note = Pepper (C2G/G31C/G10C/C30G/U6G/C27U)
  misc_feature join(1,19,20,21,32): note = n are nucleotide fragments greater than or equal to 1 in length
  source 1..32: mol_type = other RNA
ngcaagcgtcgcgtgtcgnnnactggtgcgcn

SEQ ID NO: 142 - RNA, 32 nt, synthetic construct
  misc_feature 1..32: note = Pepper (C2G/G31C/G10U/C30A/U9A/U14G/C27U)
  misc_feature join(1,19,20,21,32): note = n are nucleotide fragments greater than or equal to 1 in length
  source 1..32: mol_type = other RNA
ngcaatcgatgcgggtcgnnnactggtgcacn

SEQ ID NO: 143 - RNA, 32 nt, synthetic construct
  misc_feature 1..32: note = Pepper (C2A/G31U/G10U/C30A/U9A/U14G/C27U)
  misc_feature join(1,19,20,21,32): note = n are nucleotide fragments greater than or equal to 1 in length
  source 1..32: mol_type = other RNA
nacaatcgatgcgggtcgnnnactggtgcatn

(19) United States
(12) Patent Application Publication
(10) Pub. No.: US 2026/0357270 A1
(43) Pub. Date: Nov. 12, 2026

(54) APTAMER NUCLEIC ACID MOLECULE, AND COMPLEX AND APPLICATION THEREOF

(71) Applicant: EAST CHINA UNIVERSITY OF SCIENCE AND TECHNOLOGY, Shanghai (CN)

(72) Inventors: Yi YANG, Shanghai (CN); Linyong ZHU, Shanghai (CN); Xianjun CHEN, Shanghai (CN); Dasheng ZHANG, Shanghai (CN); Ni SU, Shanghai (CN); Qiuning LIN, Shanghai (CN)

(21) Appl. No.: 19/663,447

(22) Filed: Apr. 30, 2026

Related U.S. Application Data
(63) Continuation of application No. 17/607,102, filed on Apr. 28, 2020, now Pat. No. 12,662,676.

(30) Foreign Application Priority Data
Apr. 28, 2019 (CN) 201910348701.7

(51) Int. Cl. C12N 15/115; C07C 255/53; C07C 255/58; C07D 213/74; C07D 213/85; C07D 239/42; C07D 241/20; C07D 241/24; C07D 263/56; C07D 277/64; C07D 333/60; C07D 495/04; C09K 11/06; C12N 15/10; C12Q 1/6876

(52) U.S. Cl. CPC C12N 15/115; C07C 255/53; C07C 255/58; C07D 213/74; C07D 213/85; C07D 239/42; C07D 241/20; C07D 241/24; C07D 263/56; C07D 277/64; C07D 333/60; C07D 495/04; C09K 11/06; C12N 15/1003; C12Q 1/6876; C09K 2211/1007; C09K 2211/1018; C12N 2310/16; C12N 2310/351; C12N 2320/10

(57) ABSTRACT

The present application relates to an aptamer nucleic acid molecule, a complex containing the aptamer nucleic acid molecules, a method of detecting intracellular or extracellular RNA, DNA or other target molecules, and a kit containing the aptamer. The aptamer of the present application is capable of specifically binding a kind of fluorophore micromolecules, and can significantly enhance fluorescence intensity under excitation light of appropriate wavelength.

### CROSS REFERENCE TO RELATED APPLICATIONS

This application is a continuation application of U.S. patent application Ser. No. 17/607,102, filed on Oct. 28, 2021, which claims a priority from China Patent Application No. 201910348701.7 filed on Apr. 28, 2019.

### SEQUENCE LISTING

The official copy of sequence listing is submitted with the specification as an XML file with a file name of “SH260048-US-ST26 sequence list.xml”, a creation date of Jun. 24, 2026 and a size of 227,529 bytes. This sequence listing is part of the specification and is hereby incorporated in its entirety by reference herein.

### FIELD OF THE INVENTION

The present application relates to an aptamer nucleic acid molecule, a complex containing the aptamer nucleic acid molecules, a method of detecting intracellular or extracellular RNA, DNA or other target molecules, and a kit containing the aptamer. The aptamer of the present application is capable of specifically binding a kind of fluorophore micromolecules, and can significantly enhance fluorescence intensity under excitation light of appropriate wavelength.

### BACKGROUND OF THE INVENTION

Among all biomacromolecules, RNA exhibits the most diverse biological functions. In the Central Dogma of biology, RNA serves as the transmitter of genetic material (messenger RNA), template for protein synthesis (ribosomal RNA), and amino acid transporter (transfer RNA), constitutes a series of physiological processes, and finally realizes gene transcription and expression. In the past few decades, scientists have gradually discovered the crucial functions of RNA in a variety of vital activities, including many RNA-protein complexes, such as telomerase, splicing enzyme, ribozyme, and riboswitch or the like. In addition, some non-coding RNAs, such as short interfering RNA (siRNA), microRNA (microRNA), and long non-coding RNA (lncRNA), have played an irreplaceable role in the regulation of gene expression at the post-transcriptional level for the past few years. Real-time monitoring of RNA transport and metabolic processes in cells is critical for studying the relationship between RNA localization and gene expression, as well as cell regulation. At present, scientists have identified several mechanisms that can lead to different subcellular localizations of RNA, such as active transport, passive diffusion, and anchoring and so on. In many polar cells, especially nerve cells, the spatial specific expression of mRNA is closely related to the plasticity, learning and memory of neurons. Therefore, once these regulation processes of RNA are damaged, it will cause neuronal dysfunction and neurological diseases.

RNA fluorescence in situ hybridization technology is a method that has been widely used for a long time in the study of the level and distribution of RNA in cells, and uses molecular hybridization in the fluorescent labeling and imaging of specific RNA molecules. However, its operation is relatively complicated and contains an elution step, so it can only be used for the study of immobilized cells, i.e. dead cells, rather than for real-time monitoring of the dynamic changes of RNA in living cells. Molecular Beacon is the first developed RNA imaging technology of living cells. It is a stem-loop double-labeled oligonucleotide probe using an auto hairpin structure formed at the 5′ and 3′ ends, and, when it binds to the target RNA, the quenching effect of a quenched group labeled at one end on the fluorescence group is eliminated and the fluorescent group produces fluorescence, or the FRET of the fluorescent groups at both ends disappears. However, Molecular Beacons have disadvantages such as low fluorescence signals, difficulty in entering cells, being easily degradable, serious non-specific aggregation in the nucleus, being susceptible to RNA secondary structure and a custom oligonucleotide probes being needed for each RNA, etc., and these shortcomings limit the wide application of this technology.

The current methods of RNA imaging for living cells mainly use the MCP-FPs system, which can specifically recognize mRNA molecules fused with multiple-copies of MS2 sequences, and monitor mRNA synthesis and distribution in real time by detecting fluorescent protein signals (Ozawa et al., Nature Methods, 2007. 4: 413-419). However, due to the high background fluorescence generated by MCP-FPs not bound to mRNA molecules, the signal-to-noise ratio of this method is very low. Then, scientists added nuclear localization signals to MCP-FPs fusion protein so that GFP-MS2 not bound to mRNA molecules were localized in the nucleus, thereby reducing the non-specific fluorescence in cytoplasm to a certain extent and increasing the signal-to-noise ratio of the detection.

In addition to cellular RNA detection by means of the RNA-binding protein-fluorescent protein technology, scientists have been looking for RNA fluorescent label, which is similar to GFP, for RNA imaging. Scientists constructed a fluorophore-quencher combination, wherein the quencher cannot quench the fluorescent signal of the fluorophore when the fluorophore aptamer (Aptamer) binds to the fluorophore, and the complex formed of aptamer-fluorophore-quencher is fluorescent at this moment. When the aptamer of the fluorophore is not present, the fluorescence signal of the fluorophore will be quenched by the quencher. Based on this principle, scientists have realized the imaging of mRNA in bacteria (Arora et al., Nucleic Acids Research, 2015. 21: e144). In addition, a label named IMAGE (intracellular multi aptamer genetic) has also been developed, which is formed of two different aptamer-micromolecule complexes. When the micromolecules bind to the aptamer in RNA sequence, the fluorophore carried by two adjacent micromolecules will undergo fluorescence resonance energy transfer (FRET), and RNA in cells can be detected by the detection of changes in the fluorescence signal. However, neither of these two methods has achieved real-time monitoring of RNA in mammalian cells. In 2011, S. Jaffrey's research group obtained a nucleic acid aptamer called “Spinach”, which can specifically bind to a fluorophore (3,5-difluoro-4-hydroxybenzyli-dene imidazolinone, DFHBI) and thus significantly increases its fluorescence (Paige et al., Science, 2011. 333: 642-646; Strack et al., Nature Methods, 2013. 10: 1219-1224). “Spinach2”, which is a “Spinach” mutant, is more stable and provides an excellent tool for labeling RNA in living cells by means of genetic coding. The research group replaced a stem-loop structure in “Spinach” with a nucleic acid aptamer which can specifically bind to cell metabolites, and developed a tool that can detect cell metabolites based on Spinach-DFHBI complex (Paige et al., Science, 2012. 335: 1194). So far, this method has been successfully used to monitor and analyze the dynamic changes of RNA in bacteria, yeast and mammalian cells. Subsequently, the research group also developed a Corn-DFHO complex for the activity detection of RNA polymerase III promoter in mammalian cells (Song et al., Nature Chemical Biology, 2017. 13: 1187-1194). However, the wide application of this method has been greatly limited by the following shortcomings: (1) the binding ability of the aptamer-fluorophore complex is weak with a dissociation constant (kd) of tens to hundreds nM; (2) the fluorescence signal of the aptamer-fluorophore complex is unstable and can be easily quenched, making its fluorescence signal undetectable (Han et al., Journal of the American Chemical Society, 2013. 135: 19033-19038); (3) so far, the spectrum is only green and yellow, lacking longer wavelengths for the imaging of RNA in living animals (Song et al., Journal of the American Chemical Society, 2014. 136: 1198-1201); (4) Corn is a dimer, which may interfere with the functions of target RNA; (5) currently, there are no other aptamer-fluorophore complexes capable of simultaneously monitoring multiple RNAs in cells.

To sum up, the currently used RNA labeling technologies have their own obvious shortcomings. MCP-FPs labeling technology has strong unbound background fluorescence and low signal-to-noise ratio. The RNA labeling technology based on the complex formed of aptamer-fluorophore-quencher currently realizes the labeling of RNA only in bacteria, and has not yet realized the labeling of RNA in mammalian cells. RNA labeling technology based on single fluorophore-nucleic acid aptamer seems to be a perfect RNA labeling technology, but it has not been widely used due to the non-ideal nature of the complex formed by the current fluorophore (DFHBI, DFHBI-1T, DFHO) and nucleic acid aptamer. Therefore, more effective fluorophore-nucleic acid aptamer complexes, which can overcome the shortcomings of the previous fluorophore-nucleic acid aptamer complexes in real-time labeling of RNA or DNA in living cells, have always been needed in the scientific and industrial communities.

### SUMMARY

The technical solutions provided by the present application are as follows.

The present application provides a nucleic acid aptamer molecule, a DNA molecule encoding the nucleic acid aptamer molecule, a complex of nucleic acid aptamer molecules and fluorophore molecules, and uses of the complex.

The present application provides a nucleic acid aptamer molecule containing following nucleotide sequences (a), (b) or (c):
(a): a nucleotide sequence NiCCAAUCGUGGCGUGUCGN₁₉-N₂₀-N₂₁ACUGGCGCCGN₃₂ (called as General Formula Pepper structure), wherein N₁, N₁₉, N₂₀, N₂₁ and N₃₂ represent nucleotide fragments greater than or equal to 1 in length, and at least one base pair in N₁ and N₃₂ nucleotide sequences forms a complementary pair, and at least one base pair in N₁₉ and N₂₁ nucleotide sequences forms a complementary pair;
(b): a nucleotide sequence with an identity of at least 70% to the nucleotide sequence defined by (a); and
(c): a nucleic acid aptamer molecule derived from (a) at a position not including N₁, N₁₉, N₂₀, N₂₁ and N₃₂ in the nucleotide sequence defined by (a), with substitution, missing and/or addition of one or several nucleotides, and having an aptamer function.

In some embodiments, the nucleotide sequence (b) has an identity of at least 75%, 76%, 78%, 80%, 82%, 85%, 87%, 90%, 93%, 95%, 96%, 97%, 98%, 99% or 100% to the General Formula Pepper structure nucleotide sequence defined by the nucleotide sequence (a). In some embodiments, the nucleotide sequence (c) is nucleic acid aptamer molecules obtained with substitution, missing and/or addition of 10, 9, 8, 7, 6, 5, 4, 3, 2 or one nucleotide at a position in the General Formula Pepper structure nucleotide sequence defined by the nucleotide sequence (a) and not including N₁, N₁₉, N₂₀, N₂₁ and N₃₂. In some embodiments, the nucleotide sequence (c) is nucleic acid aptamer molecules obtained with substitution, missing and/or addition of 7, 6, 5, 4, 3, 2 or one nucleotide at a position in the General Formula Pepper structure nucleotide sequence defined by the nucleotide sequence (a) and not including N₁, N₁₉, N₂₀, N₂₁ and N₃₂.

In some embodiments, when N₁ and N₃₂ in the nucleotide sequence (a) form a complementary pair, a direction of N₁ nucleotide sequence is 5′-3′, and a direction of N₃₂ nucleotide sequence is 3′-5′; and when N₁₉ and N₂₁ form a complementary pair, a direction of N₁₉ nucleotide sequence is 5′-3′, and a direction of N₂₁ nucleotide sequence is 3′-5′.

In some embodiments, when at least one fragment of N₁ and N₃₂ in the nucleotide sequence (a) is greater than or equal to 5 nucleotide bases in length, at least two base pairs in N₁ and N₃₂ nucleotide sequences form complementary pairs; when at least one fragment of N₁₉ and N₂₁ is greater than or equal to 5 nucleotide bases in length, at least two base pairs in N₁₉ and N₂₁ nucleotide sequences form complementary pairs.

In some embodiments, the nucleotide substitution in the General Formula Pepper structure is selected from one of the following groups: C3A, C3U, A4U, A4G, A4C, A5G, A5C, U6A, U6G, U6C, C7A, C7U, G8C, U9A, G11A, G11U, C12G, C12A, C12U, G13C, U14A, U14G, C17U, G18U, G18C, C27G, C27U, G28U, C29G, C29U, C30A, C30U, C2G/G31C, C2U/G31A, C2A/G31U, G10A/C30U, G10C/C30G, G10U/C30A, C2G/G31C/C3A, C2G/G31C/A4C, C2G/G31C/A5C, C2G/G31C/G8C, C2G/G31C/C12U, C2G/G31C/U14G, C2G/G31C/C27U, C2G/G31C/C29G, C2G/G31C/C30U, C2G/G31C/G10A/C30U, C2G/G31C/G10C/C30G, C2G/G31C/G10U/C30A, C2U/G31A/G10A/C30U, C2U/G31A/G10C/C30G, C2U/G31A/G10U/C30A, C2A/G31U/G10A/C30U, C2A/G31U/G10C/C30G, C2A/G31U/G10U/C30A, C2G/G31C/G10C/C30G/C3A, C2G/G31C/G10C/C30G/A4C, C2G/G31C/G10C/C30G/A5C, C2G/G31C/G10C/C30G/G8C, C2G/G31C/G10C/C30G/C12U, C2G/G31C/G10C/C30G/U14G, C2G/G31C/G10C/C30G/C27U, C2G/G31C/G10C/C30G/C29G, C2G/G31C/G10A/C30U/U6G/C27U, C2G/G31C/G10C/C30G/U6G/C27U, C2G/G31C/G10U/C30A/U9A/U14G/C27U and C2A/G31U/G10U/C30A/U9A/U14G/C27U.

In some embodiments, the nucleotide substitution in the General Formula Pepper structure is selected from one of the following groups: C3A, C3U, A4C, A5C, C7U, G8C, U9A, C12G, C12U, U14G, C27U, C29G, C30U, C2G/G31C, C2U/G31A, C2A/G31U, G10A/C30U, G10C/C30G, G10U/C30A, C2G/G31C/C3A, C2G/G31C/A4C, C2G/G31C/A5C, C2G/G31C/G8C, C2G/G31C/C12U, C2G/G31C/U14G, C2G/G31C/C27U, C2G/G31C/C29G, C2G/G31C/C30U, C2G/G31C/G10A/C30U, C2G/G31C/G10C/C30G, C2G/G31C/G10U/C30A, C2U/G31A/G10A/C30U, C2U/G31A/G10C/C30G, C2U/G31A/G10U/C30A, C2A/G31U/G10A/C30U, C2A/G31U/G10C/C30G, C2A/G31U/G10U/C30A, C2G/G31C/G10C/C30G/C3A, C2G/G31C/G10C/C30G/A4C, C2G/G31C/G10C/C30G/A5C, C2G/G31C/G10C/C30G/G8C, C2G/G31C/G10C/C30G/C12U, C2G/G31C/G10C/C30G/U14G, C2G/G31C/G10C/C30G/C27U, C2G/G31C/G10C/C30G/C29G, C2G/G31C/G10A/C30U/U6G/C27U and C2G/G31C/G10C/C30G/U6G/C27U.

In some embodiments, the nucleotide substitution in the General Formula Pepper structure is selected from one of the following groups: C3A, C3U, A4C, A5C, C7U, G8C, U9A, C12G, C12U, U14G, C27U, C29G, C30U, C2G/G31C, C2U/G31A, C2A/G31U, G10A/C30U, G10C/C30G, G10U/C30A, C2G/G31C/C3A, C2G/G31C/A4C, C2G/G31C/A5C, C2G/G31C/G8C, C2G/G31C/C12U, C2G/G31C/U14G, C2G/G31C/C27U, C2G/G31C/C29G, C2G/G31C/C30U, C2G/G31C/G10A/C30U, C2G/G31C/G10C/C30G, C2G/G31C/G10U/C30A, C2U/G31A/G10A/C30U, C2U/G31A/G10C/C30G, C2U/G31A/G10U/C30A, C2A/G31U/G10A/C30U, C2A/G31U/G10C/C30G and C2A/G31U/G10U/C30A.

In some embodiments, nucleotide sequences at N₁ and N₃₂ in the nucleotide sequence (a) are F30 or tRNA scaffold RNA sequences.

In some embodiments, the nucleic acid aptamer molecules are RNA molecules or RNA base-modified molecules.

In some embodiments, the nucleic acid aptamer molecules are DNA-RNA hybrid molecules or DNA-RNA base-modified molecules.

In some embodiments, N₁₉-N₂₀-N₂₁ in the nucleotide sequence (a) contains a nucleotide sequence capable of identifying target molecules.

In some embodiments, the target molecules include but are not limited to: proteins, nucleic acid, lipid molecules, carbohydrates, hormones, cytokines, chemokines, and metabolite metal ions.

In some embodiments, N₁₉-N₂₀-N₂₁ in the nucleotide sequence (a) is a nucleotide sequence capable of identifying GTP and adenosine molecules.

In some embodiments, the aptamer function refers to that the nucleic acid aptamer can enhance fluorescence intensity of fluorophore molecules under excitation light of appropriate wavelength by at least two times, at least 5 to 10 times, at least 20 to 50 times, at least 100 to 200 times or at least 500 to 1,000 times.

In some embodiments, the nucleic acid aptamer molecules may further include concatemers that can bind multiple fluorophore molecules, and the concatemers are connected by spacer sequences of appropriate length having 2, 3, 4, 5, 6, 7, 8 or more nucleotide fragments. Nucleotides of the concatemers can be selected from but are not limited to a sequence SEQ ID No: 8, 9, 10, 11, 12, 13, 14, 15, 16, 17, 18 and 19.

The present application further provides a complex of nucleic acid aptamer molecules and fluorophore molecules, wherein the nucleic acid aptamer molecules are any kind of the afore-mentioned nucleic acid aptamer molecules, and the fluorophore molecules have a structure shown in Formula (I) below:

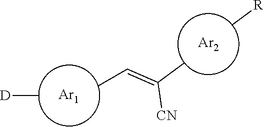

wherein: D- is X₁O— or N(X₂)(X₃)—; X₁, X₂, X₃ are respectively and independently selected from hydrogen, straight or branched alkyl and modified alkyl with 1 to 10 carbons, and X₂ and X₃ are optionally interconnected to be a satured or unsatured ring; R— is selected from hydrogen, cyano group, carboxyl, amide group, ester group, hydrixy, and straight or branched alkyl or modified alkyl with 1 to 10 carbons; Ar₁ and Ar₂ are respectively and independently selected from monocyclic aryl subunits, monocyclic heteraryl subunits, or aromatic subunits with 2 to 3 ring structures condensed from one or both of monocyclic aryl group and monocyclic heteroaryl group;
wherein: hydrogen atoms in Ar₁, and Ar₂ can be independently substituted by F, Cl, Br, I, hydroxyl group, nitro group, aldehyde group, carboxyl group, cyano group, sulfonic acid group, sulfuric acid group, phosphoric acid group, amino group, primary amino group, secondary amino group, straight or branched alkyl and modified alkyl with 1 to 10 carbons;
wherein: the modified alkyl is a group obtained by replacing any carbon atom of the alkyl with at least one group selected from F, Cl, Br, I, —O—, —OH, —CO—, —NO₂, —CN, —S—, —SO₂—, —(S═O)—, azide group, phenylene, primary amino group, secondary amino group, tertiary amino group, quaternary ammonium group, ethylene oxide, succinate, isocyanate, isothiocyanate, acyl chloride, sulfonyl chloride, saturated or unsaturated monocyclic or dicyclic subcyclic hydroxyl, bridged ester heterocycle, and the modified alkyl has 1 to 10 carbon atoms, wherein the carbon-carbon single bond is optionally and independently replaced by a carbon-carbon double bond or a carbon-carbon triple bond; and
wherein the nucleic acid aptamer molecules and the fluorophore molecules in the complex respectively exist in an individual solution, or the nucleic acid aptamer molecules and the fluorophore molecules are in the same solution.

In some embodiments, the modified alkyl contains at least one group selected from —OH, —O—, glycol unit, monosaccharide unit, disaccharide unit, —O—CO—, —NH—CO—, —SO₂—O—, —SO—, Me₂N—, Et₂N—, —S—S—, —CH═CH—, F, Cl, Br, I, —NO₂ and cyano group.

In some embodiments, aromatic rings contained in the fluorophore molecules are selected from structures represented by the following formulae (II-1) to (II-15):

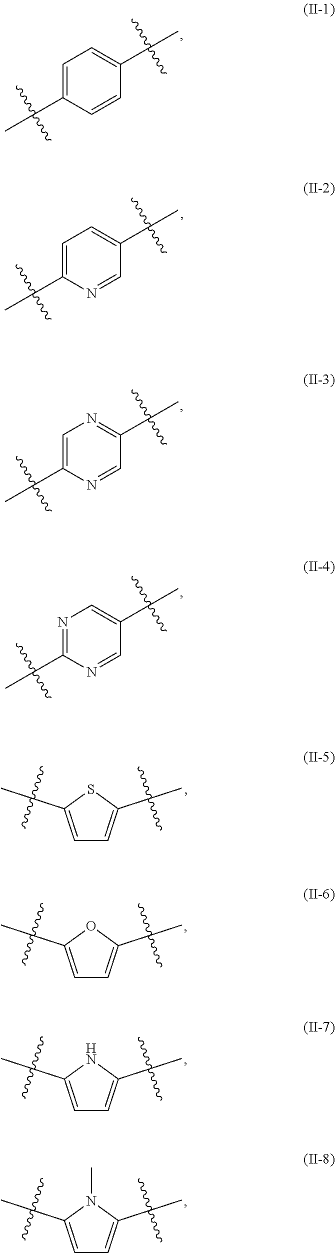

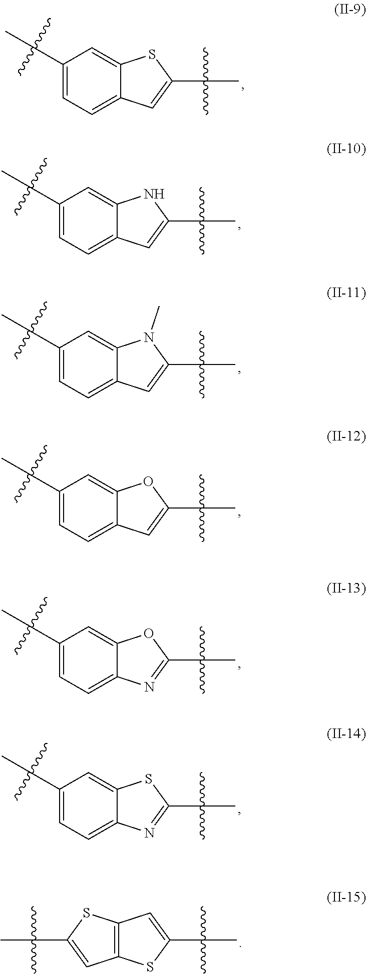

In some embodiments, fluorophore molecules are selected from the following compounds represented by the following formulae:

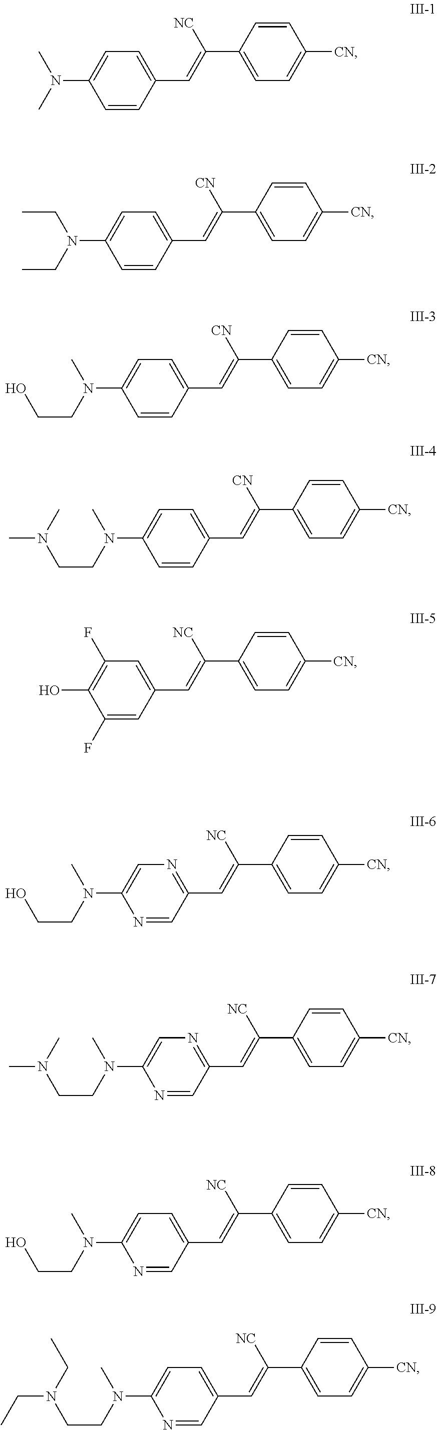

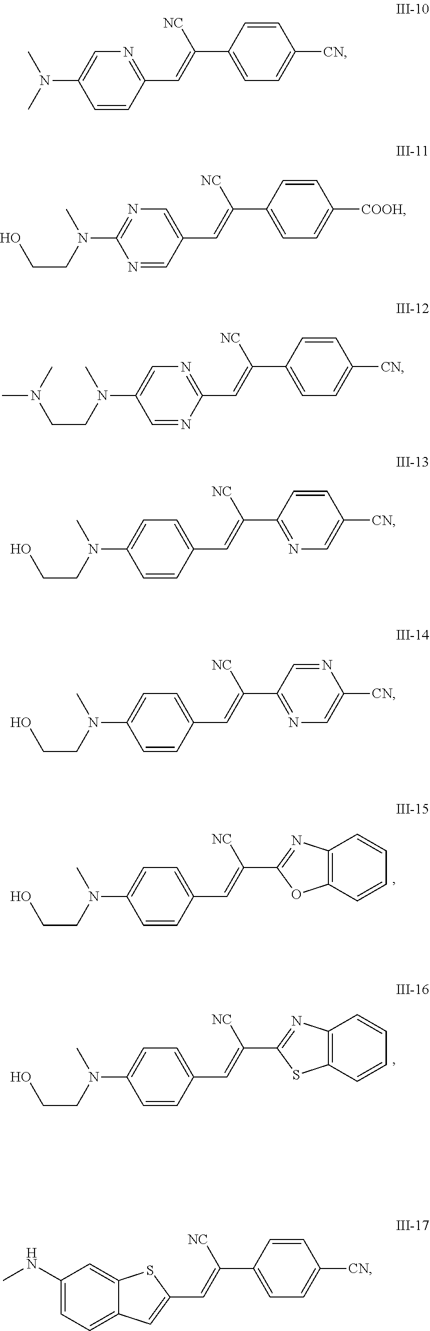

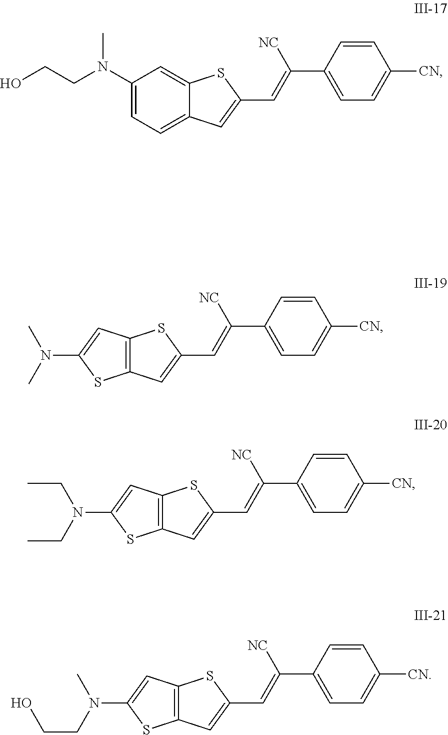 [SMILES: CN(CCO)c1ccc2cc(/C=C(\C#N)c3ccc(C#N)cc3)sc2c1 ; CN(C)c1cc2sc(/C=C(\C#N)c3ccc(C#N)cc3)cc2s1 ; CCN(CC)c1cc2sc(/C=C(\C#N)c3ccc(C#N)cc3)cc2s1 ; CN(CCO)c1cc2sc(/C=C(\C#N)c3ccc(C#N)cc3)cc2s1]

In some embodiments, the fluorophore molecules in the complex are selected from III-1, III-2, III-3, III-4, III-5, III-6, III-7, III-8, III-9, III-10, III-11, III-12, III-13, III-14, III-15, III-16, III-17, III-18, III-19, III-20 and III-21.

In some embodiments, the aptamer molecules in the complex contain nucleotide sequence SEQ ID No: 1, 2, 3, 6, 7, 8, 9, 10, 11, 12, 13, 14, 15, 16, 17, 18, 19, 21, 22, 23, 24, 25, 26, 27, 28, 29, 30 or 31.

The present application further provides any of the afore-mentioned complexes used for detection or labeling of target nucleic acid molecules in vitro or in vivo.

The present application further provides any of the afore-mentioned complexes used for detection or labeling of extracellular or intracellular target molecules.

The present application further provides any of the afore-mentioned complexes used for imaging of genomic DNA.

The present application further provides any of the afore-mentioned complexes used for detecting a relationship between mRNA and protein content in cells.

The present application further provides a DNA molecule which transcribes any of the nucleic acid aptamer molecules.

The present application further provides an expression vector containing the DNA molecules.

The present application further provides a host cell containing the expression vector.

The present application further provides a kit containing any of the nucleic acid aptamer molecules and/or any of the expression vectors and/or any of the host cells and/or any of the complexes.

The present application further provides a method of detecting target molecules, including:
adding any of the complexes to a solution containing the target molecules;
exciting the complex with light of appropriate wavelength; and
detecting fluorescence of the complex.

The present application further provides a method of detecting genome DNA, including imaging genome DNA with any of the complexes.

The present application further provides a method of extracting and purifying RNA, including extracting and purifying RNA with any of the complexes.

The inventor designed brand new nucleic acid aptamer molecules, and synthesized brand new fluorophore molecules, so as to form a brand new fluorophore-nucleic acid aptamer complex. After binding of the aptamer molecules to the fluorophore molecules, the fluorescence intensity of the fluorophore molecules can be significantly increased under the excitation light of appropriate wavelength. They overcome the shortcomings of previous fluorophore-nucleic acid aptamer complexes, and can be used for effective real-time RNA/DNA labeling in living cells. The nucleic acid aptamer of the present application have a strong affinity for fluorophore molecules, and shows different fluorescence spectra and fine light and temperature stability. These nucleic acid aptamer-fluorophore molecule complexes can be used for real-time labeling and imaging of RNA/DNA in prokaryotic and eukaryotic cells, detecting protein-RNA interactions, exploring the relationship between mRNA content and protein in cells, or as labels for RNA extraction and purification.

### BRIEF DESCRIPTION OF THE FIGURES

FIG. 1 Secondary structure prediction of nucleic acid aptamer molecules. (A) is the predicted general structure of Pepper, comprising N₁ and N₃₂ that can form a stem structure, and N₁₉, N₂₀ and N₂₁ that can form a stem-loop structure. (B) is the predicted general structure of Pepper-1, wherein the base sequence of N₁ and N₃₂ is shown in the dotted box corresponding to Stem 1 in the drawing, and the base sequence of N₁₉, N₂₀ and N₂₁ is shown in the dotted box corresponding to the stem-loop.

FIG. 2 Secondary structure prediction of F30-Pepper-1.

FIG. 3 Secondary structure prediction of tRNA-Pepper-2.

FIG. 4 Nature identification of F30-Pepper-1-III-3 complex. (A) Fluorescence excitation spectrum and emission spectrum of F30-Pepper-1-III-3 complex; (B) absorption spectra of F30-Pepper-1-III-3 complex and III-3; (C) oligomerization identification of F30-Pepper-1-III-3 complex; the “ruler” is a single-stranded DNA standard used for calibrating size of the aptamer; (D) dissociation constant determination of the binding of F30-Pepper-1 to III-3; (E) temperature stability determination of F30-Pepper-1-III-3 complex; (F) pH stability determination of F30-Pepper-1-III-3 complex; (G) determination of dependence of F30-Pepper-1-III-3 complex on K+.

FIG. 5 Activation effect of Pepper modified with different bases on III-3. (A) The secondary structure diagram of Pepper-3 aptamers containing deoxyribonucleotides (dark color in the drawing); (B) the secondary structure diagram of Pepper-4 aptamers with 2′F modification (dark color in the drawing); (C) activation effects of Pepper with different modifications on III-3. “Control” is to replace Pepper-3 or Pepper-4 aptamer with buffer.

FIG. 6 Activation effects of different Pepper concatemers on III-3. (A) Obtaining Pepper concatemers in a way of “Tandem 1”; (B) obtaining Pepper concatemers in a way of “Tandem 2”; (C) obtaining Pepper concatemers in a way of “Tandem 3”; (D) activation effects of different Pepper concatemers obtained in the way of “Tandem 1” on III-3; (E) activation effects of different Pepper concatemers obtained in the way of “Tandem 2” on III-3; (F) activation effects of different Pepper concatemers obtained in the way of “Tandem 3” on III-3.

FIG. 7 The labeling effect of F30-Pepper-1-III-3 complex on RNA in bacteria.

FIG. 8 The labeling effect of F30-Pepper-1-III-3 complex on RNA in yeast cells.

FIG. 9 The labeling effects of Pepper, III-3 and analogue thereof in labeling RNA in mammalian cells. (A) Comparison of the effects of F30-Pepper-1-III-3, F30-Broccoli-DFHBI-1T and tRNA-Corn-DFHO on RNA labeling in mammalian cell; (B) statistical results of fluorescence in Fig. (A); (C) effects of F30-8Pepper-5 and III-3 analogs on RNA labeling in mammalian cells.

FIG. 10 Probe construction based on Pepper-1. (A) Diagram of probe construction, wherein the stem-loop structure can identify adenosine or GTP; (B) detection effect of adenosine probe; (C) detection effect of GTP probe.

FIG. 11 RNA localization in tracer cells by using Pepper. (A) Detection of GAPDH mRNA localization by using Pepper; (B) detection of Pepper TMED2 mRNA localization by using Pepper.

FIG. 12 Exploration of the relationship between mRNA and protein in cells by using Pepper. (A) Flow cytometry analysis result of BFP protein and RNA expression thereof, (B) flow cytometry analysis result of mCherry protein and RNA expression thereof.

FIG. 13 Detection of genomic DNA by using Pepper. (A) Diagram of dCas9 and different chimeric sgRNA; (B) imaging results of genomic DNA by dCas9 and different chimeric sgRNA; (C) statistical results of bright spot particles in each cell in (B).

FIG. 14 Super-resolution imaging of RNA by using Pepper. (A) Co-localization of 4Pepper-9-MS2 RNA and tdMCP-BFP-H2B protein; (B) imaging results of wide field and SIM in nuclear middle layer; (C) imaging results of wide field and SIM in nuclear top layer.

FIG. 15 Pepper serving as labels for RNA extraction and purification. The “ruler” is a single-stranded DNA standard used for calibrating size of the aptamer.

### EXAMPLES

The following definitions and embodiments cited in the present application will be described in details here. The contents of all patents and published literature referred to herein, including all sequences disclosed in these patents and published literature, are expressly incorporated herein by reference. Hereinafter, “nucleotides” and “nucleotide bases” are used interchangeably and stand for the same meaning.

Following are detailed explanations of some terms used in the present application.

### Nucleic Acid Aptamer Molecules

The “nucleic acid aptamer molecules” of the present application are also referred to as “aptamer molecules”. The nucleic acid aptamer molecule contains (a) a nucleotide sequence NiCCAAUCGUGGCGUGUCGN₁₉-N₂₀-N₂₁ACUGGCGCCGN₃₂ (corresponding to the General Formula Pepper structure in FIG. 1A); or (b) which is a nucleotide sequence with an identity of at least 70% to the nucleotide sequence of (a); wherein at least one base pair in N₁ and N₃₂ nucleotide sequences forms a reverse complementary pair, namely, the direction of N₁ nucleotide sequence is 5′-3′, and the direction of N₃₂ nucleotide sequence is 3′-5′. When the length of at least one nucleotide base of N₁ and N₃₂ is smaller than or equal to 4, at least one base pair is needed for forming the complementary pair; and when the length of at least one nucleotide base of N₁ and N₃₂ is greater than or equal to 5, at least two base pairs are needed for forming the complementary pair. Wherein, at least one base pair in N₁₉ and N₂₁ nucleotide sequences forms a reverse complementary pair, namely, the direction of N₁₉ nucleotide sequence is 5′-3′, and the direction of N₂₁ nucleotide sequence is 3′-5′. When the length of at least one nucleotide base of N₁₉ and N₂₁ is smaller than or equal to 4, at least one base pair is needed for forming the complementary pair; and when the length of at least one nucleotide base of N₁₉ and N₂₁ is greater than or equal to 5, at least two base pairs are needed for forming the complementary pair. N₂₀ therein is a nucleotide base of any length or composition; or (c) which is at any position in the nucleotide sequence (a) with the substitution, missing and/or addition of 1 to 7 nucleotides.

The nucleic acid aptamer molecules contain substitution of the nucleotides in General Formula Pepper structure, the substitution being selected from one of the following groups: C3A, C3U, A4U, A4G, A4C, A5G, A5C, U6A, U6G, U6C, C7A, C7U, G8C, U9A, G11A, G11U, C12G, C12A, C12U, G13C, U14A, U14G, C17U, G18U, G18C, C27G, C27U, G28U, C29G, C29U, C30A, C30U, C2G/G31C, C2U/G31A, C2A/G31U, G10A/C30U, G10C/C30G, G10U/C30A, C2G/G31C/C3A, C2G/G31C/A4C, C2G/G31C/A5C, C2G/G31C/G8C, C2G/G31C/C12U, C2G/G31C/U14G, C2G/G31C/C27U, C2G/G31C/C29G, C2G/G31C/C30U, C2G/G31C/G10A/C30U, C2G/G31C/G10C/C30G, C2G/G31C/G10U/C30A, C2U/G31A/G10A/C30U, C2U/G31A/G10C/C30G, C2U/G31A/G10U/C30A, C2A/G31U/G10A/C30U, C2A/G31U/G10C/C30G, C2A/G31U/G10U/C30A, C2G/G31C/G10C/C30G/C3A, C2G/G31C/G10C/C30G/A4C, C2G/G31C/G10C/C30G/A5C, C2G/G31C/G10C/C30G/G8C, C2G/G31C/G10C/C30G/C12U, C2G/G31C/G10C/C30G/U14G, C2G/G31C/G10C/C30G/C27U, C2G/G31C/G10C/C30G/C29G, C2G/G31C/G10A/C30U/U6G/C27U, C2G/G31C/G10C/C30G/U6G/C27U, C2G/G31C/G10U/C30A/U9A/U14G/C27U and C2A/G31U/G10U/C30A/U9A/U14G/C27U (which are the aptamer molecule structures in Table 1). These mutants can specifically bind fluorophore molecules, and can significantly increase fluorescence intensity of fluorophore molecules under excitation light of appropriate wavelength after binding. The nucleotide position sequence corresponds to the position shown in FIG. 1A.

The afore-mentioned mutants indicate that nucleotide substitution occurs at the corresponding sites of the aptamer nucleotide sequence of the General Formula Pepper structure. For example, C3A indicates that the cytosine nucleotide C at the third position of Pepper is substituted by adenine nucleotide A, i.e. Pepper (C3A) in Table 1; C2G/G31C indicates that C at the second position of Pepper is substituted by G, and G at the 31^{st} position is substituted by C, i.e. Pepper (C2G/G31C) in Table 1.

TABLE 1
Aptamer structure of Pepper general formula structure after
substitution of 7, 6, 5, 4, 3, 2 or one nucleotide

| Substitutions of | Aptamer structure general formula |
|---|---|
| Pepper general | after substitution |
| formula structure | (underlined are bases after substitution) |
| Pepper (C3A) | N₁CAAAUCGUGGCGUGUCGN₁₉-N₂₀- |
| Pepper (C3U) | N₁CUAAUCGUGGCGUGUCGN₁₉-N₂₀- |
| Pepper (A4U) | N₁CCUAUCGUGGCGUGUCGN₁₉-N₂₀- |
| Pepper (A4G) | N₁CCGAUCGUGGCGUGUCGN₁₉-N₂₀- |
| Pepper (A4C) | N₁CCCAUCGUGGCGUGUCGN₁₉-N₂₀- |
| Pepper (A5G) | N₁CCAGUCGUGGCGUGUCGN₁₉-N₂₀- |
| Pepper (A5C) | N₁CCACUCGUGGCGUGUCGN₁₉-N₂₀- |
| Pepper (U6A) | N₁CCAAACGUGGCGUGUCGN₁₉-N₂₀- |
| Pepper (U6G) | N₁CCAAGCGUGGCGUGUCGN₁₉-N₂₀- |
| Pepper (U6C) | N₁CCAACCGUGGCGUGUCGN₁₉-N₂₀- |
| Pepper (C7A) | N₁CCAAUAGUGGCGUGUCGN₁₉-N₂₀- |
| Pepper (C7U) | N₁CCAAUUGUGGCGUGUCGN₁₉-N₂₀- |
| Pepper (G8C) | N₁CCAAUCCUGGCGUGUCGN₁₉-N₂₀- |
| Pepper (U9A) | N₁CCAAUCGAGGCGUGUCGN₁₉-N₂₀- |
| Pepper (G11A) | N₁CCAAUCGUGACGUGUCGN₁₉-N₂₀- |
| Pepper (G11U) | N₁CCAAUCGUGUCGUGUCGN₁₉-N₂₀- |
| Pepper (C12G) | N₁CCAAUCGUGGGGUGUCGN₁₉-N₂₀- |
| Pepper (C12A) | N₁CCAAUCGUGGAGUGUCGN₁₉-N₂₀- |
| Pepper (C12U) | N₁CCAAUCGUGGUGUGUCGN₁₉-N₂₀- |
| Pepper (G13C) | N₁CCAAUCGUGGCCUGUCGN₁₉-N₂₀- |
| Pepper (U14A) | N₁CCAAUCGUGGCGAGUCGN₁₉-N₂₀- |
| Pepper (U14G) | N₁CCAAUCGUGGCGGGUCGN₁₉-N₂₀- |
| Pepper (C17U) | N₁CCAAUCGUGGCGUGUUGN₁₉-N₂₀- |
| Pepper (G18U) | N₁CCAAUCGUGGCGUGUCUN₁₉-N₂₀- |
| Pepper (G18C) | N₁CCAAUCGUGGCGUGUCCN₁₉-N₂₀- |
| Pepper (C27G) | N₁CCAAUCGUGGCGUGUCGN₁₉-N₂₀- |
| Pepper (C27U) | N₁CCAAUCGUGGCGUGUCGN₁₉-N₂₀- |
| Pepper (G28U) | N₁CCAAUCGUGGCGUGUCGN₁₉-N₂₀- |
| Pepper (C29G) | N₁CCAAUCGUGGCGUGUCGN₁₉-N₂₀- |
| Pepper (C29U) | N₁CCAAUCGUGGCGUGUCGN₁₉-N₂₀- |
| Pepper (C30A) | N₁CCAAUCGUGGCGUGUCGN₁₉-N₂₀- |
| Pepper (C30U) | N₁CCAAUCGUGGCGUGUCGN₁₉-N₂₀- |
| Pepper (C2G/G31C) | N₁GCAAUCGUGGCGUGUCGN₁₉-N₂₀- |
| Pepper (C2U/G31A) | N₁UCAAUCGUGGCGUGUCGN₁₉-N₂₀- |
| Pepper (C2A/G31U) | N₁ACAAUCGUGGCGUGUCGN₁₉-N₂₀- |
| Pepper (C10A/C30U) | N₁CCAAUCGUAGCGUGUCGN₁₉-N₂₀- |
| Pepper (C10C/C30G) | N₁CCAAUCGUCGCGUGUCGN₁₉-N₂₀- |
| Pepper (C10U/C30A) | N₁CCAAUCGUUGCGUGUCGN₁₉-N₂₀- |
| Pepper (C2G/G31/C3A) | N₁GAAAUCGUGGCGUGUCGN₁₉-N₂₀- |
| Pepper (C2G/G31C/A4C) | N₁GCCAUCGUGGCGUGUCGN₁₉-N₂₀- |
| Pepper (C2G/G31C/A5C) | N₁GCACUCGUGGCGUGUCGN₁₉-N₂₀- |
| Pepper (C2G/G31C/G8C) | N₁GCAAUCCUGGCGUGUCGN₁₉-N₂₀- |
| Pepper (C2G/G31C/C12U) | N₁GCAAUCGUGGUGUGUCGN₁₉-N₂₀- |
| Pepper (C2G/G31C/U14G) | N₁GCAAUCGUGGCGGGUCGN₁₉-N₂₀- |
| Pepper (C2G/G31C/C27U) | N₁GCAAUCGUGGCGUGUCGN₁₉-N₂₀- |
| Pepper (C2G/G31C/C29G) | N₁GCAAUCGUGGCGUGUCGN₁₉-N₂₀- |
| Pepper (C2G/G31C/C30U) | N₁GCAAUCGUGGCGUGUCGN₁₉-N₂₀- |
| Pepper (C2G/G31C/G10A/ | N₁GCAAUCGUAGCGUGUCGN₁₉-N₂₀-N₂₁ACUGGCGCUCN₃₂ |
| C30U) | |
| Pepper (C2G/G31C/G10C/ | N₁GCAAUCGUCGCGUGUCGN₁₉-N₂₀-N₂₁ACUGGCGCGCN₃₂ |
| C30G) | |
| Pepper (C2G/G31C/G10U/ | N₁GCAAUCGUUGCGUGUCGN₁₉-N₂₀-N₂₁ACUGGCGCACN₃₂ |
| C30A) | |
| Pepper (C2U/G31A/G10C/ | N₁UCAAUCGUCGCGUGUCGN₁₉-N₂₀-N₂₁ACUGGCGCGAN₃₂ |
| C30G) | |
| Pepper (C2U/G31A/G10U/ | N₁UCAAUCGUUGCGUGUCGN₁₉-N₂₀-N₂₁ACUGGCGCAAN₃₂ |
| C30A) | |
| Pepper (C2A/G31U/G10A/ | N₁ACAAUCGUAGCGUGUCGN₁₉-N₂₀₋N₂₁ACUGGCGCUUN₃₂ |
| C30U) | |
| Pepper (C2A/G31U/G10C/ | N₁ACAAUCGUCGCGUGUCGN₁₉-N₂₀-N₂₁ACUGGCGCGUN₃₂ |
| C30G) | |
| Pepper (C2A/G31U/G10U/ | N₁ACAAUCGUUGCGUGUCGN₁₉-N₂₀-N₂₁ACUGGCGCAUN₃₂ |
| C30A) | |
| Pepper (C2G/G31C/G10C/ | N₁GAAAUCGUCGCGUGUCGN₁₉-N₂₀-N₂₁ACUGGCGCGCN₃₂ |
| C30G/C3A) | |
| Pepper (C2G/G31C/G10C/ | N₁GCCAUCGUCGCGUGUCGN₁₉-N₂₀-N₂₁ACUGGCGCGCN₃₂ |
| C30G/A4C) | |
| Pepper (C2G/G31C/G10C/ | N₁GCACUCGUCGCGUGUCGN₁₉-N₂₀-N₂₁ACUGGCGCGCN₃₂ |
| C30G/A5C) | |
| Pepper (C2G/G31C/G10C/ | N₁GCAAUCCUCGCGUGUCGN₁₉-N₂₀-N₂₁ACUGGCGCGCN₃₂ |
| C30G/G8C) | |
| Pepper (C2G/G31C/G10C/ | N₁GCAAUCGUCGUGUGUCGN₁₉-N₂₀-N₂₁ACUGGCGCGCN₃₂ |
| C30G/C12U) | |
| Pepper (C2G/G31C/G10C/ | N₁GCAAUCGUCGCGGGUCGN₁₉-N₂₀-N₂₁ACUGGCGCGCN₃₂ |
| C30G/U14G) | |
| Pepper (C2G/G31C/G10C/ | N₁GCAAUCGUCGCGGGUCGN₁₉-N₂₀-N₂₁ACUGGUGCGCN₃₂ |
| C30G/C27U) | |
| Pepper (C2G/G31C/G10C/ | N₁GCAAUCGUCGCGGGUCGN₁₉-N₂₀-N₂₁ACUGGCGGGCN₃₂ |
| C30G/C29G) | |
| Pepper (C2G/G31C/G10C/ | N₁GCAAUCGUCGCGGGUCGN₁₉-N₂₀-N₂₁ACUGGCGGGCN₃₂ |
| C30G/C29G) | |
| Pepper (C2G/G31C/G10A/ | N₁GCAAGCGUAGCGUGUCGN₁₉-N₂₀-N₂₁ACUGGUGCUCN₃₂ |
| C30U/U6G/C27U) | |
| Pepper (C2G/G31C/G10C/ | N₁GCAAGCGUCGCGUGUCGN₁₉-N₂₀-N₂₁ACUGGUGCGCN₃₂ |
| C30G/U6G/C27U) | |
| Pepper (C2G/G31C/G10U/ | N₁GCAAUCGAUGCGGGUCGN₁₉-N₂₀-N₂₁ACUGGUGCACN₃₂ |
| C30A/U9A/U14G/C27U) | |
| Pepper (C2A/G31U/G10U/ | N₁ACAAUCGAUGCGGGUCGN₁₉-N₂₀-N₂₁ACUGGUGCAUN₃₂ |
| C30A/U9A/U14G/C27U) | |

Aptamer molecules are single-stranded nucleic acid molecules that have a secondary structure (FIG. 1) consisting of one or more base pairing regions (stem) and one or more unpaired regions (loop). The nucleic acid molecules of the present application contain a secondary structure as predicted in FIG. 1. The secondary structure comprises two loop structures, two stem structures and a stem-loop structure, wherein Stem 1 plays the role of stabilizing the entire nucleic acid aptamer molecule structure, and can be substituted by other nucleotide base pairs of any length and any composition that can form stem structures. The 5′ end or 3′ end of Stem 1 structure can be fused with any objective RNA molecule for extracellular or intracellular detection of the target RNA molecules. In a preferable embodiment of the present application, the 5′ end of the nucleic acid aptamer molecule is fused with a 5S RNA sequence (Genebank: NR_023377.1); in another preferable embodiment of the present application, the 5′ end of the nucleic acid aptamer molecule is fused with a GAPDH RNA sequence (Genebank: BC009081).

The stem-loop structure in FIG. 1 plays the role of stabilizing the entire nucleic acid aptamer molecule structure, and can be replaced with other nucleotide base pairs of any length and any composition that can form stem-loop structures. The aptamer molecules of the present application may also contain other nucleotide sequences that can be inserted into the position of N₁₉-N₂₀-N₂₁, wherein the inserted nucleotide sequence replaces the stem-loop structure in FIG. 1A. The nucleotide sequence can specifically identify/bind target molecules. In the absence of target molecules, the binding of aptamer molecules and fluorophore molecules is weak, as a result of which the fluorophore molecules show weak fluorescence light; in the presence of target molecules, the binding of target molecules and the aptamer will promote binding of the aptamer and fluorophore molecules, and thus can significantly enhance the fluorescence of fluorophore molecules under excitation light of appropriate wavelength. The target molecules can be small molecules, and signal molecules on the cell surface, etc. These nucleic acid aptamers bind to specific target molecules through non-covalent binding, which mainly depends on intermolecular ionic forces, dipole force, hydrogen bonds, Van der Waals forces, positron and negative electron interactions, stacking or the combination of the above forces. The stem-loop structure can be replaced with an RNA sequence that identifies the target molecules for extracellular or intracellular detection of the target molecules. In a preferable embodiment of the present application, the stem-loop structure of aptamer molecules can bind GTP molecules; in another preferable embodiment of the present application, the stem-loop structures can bind adenosine molecules.

In a preferable embodiment of the present application, the nucleic acid aptamer molecules are preferably SEQ ID NO: 1, 2, 3, 6, 7, 8, 9, 10, 11, 12, 13, 14, 15, 16, 17, 18, 19, 21, 22 or 23, or their mutation sequence which can bind fluorophore molecules so as to significantly enhance the fluorescence of fluorophore molecules under excitation light of appropriate wavelength.

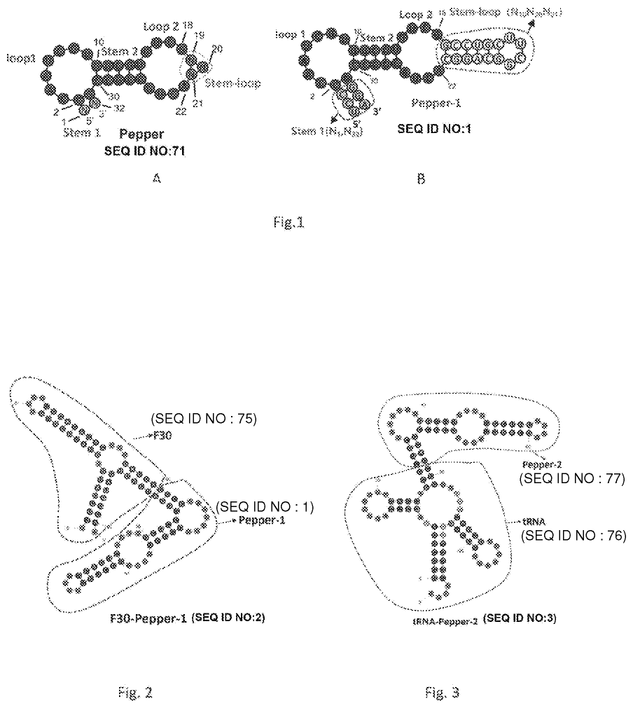

The nucleic acid aptamer molecules of the present application can also comprise a fragment of nucleotide sequence that increases its stability. In a preferable embodiment of the present application, F30 scaffold RNA (Sequence 2) was adopted, and its connection mode with the nucleic acid aptamer molecules is shown in FIG. 2; in another preferable embodiment of the present invention, tRNA scaffold RNA (Sequence 3) was adopted, and its connection mode with the nucleic acid aptamer molecules is shown in FIG. 3.

The “nucleic acid aptamer molecules” in the present application are RNA molecules, or DNA-RNA hybrid molecules with part of nucleotides being replaced with deoxyribonucleotides, wherein the nucleotides can be in a form of D and L enantiomers thereof and also contain derivatives thereof, including but not limited to 2′-F, 2′-amino, 2′-methoxyl, 5′-iodo, 5′-bromine-modified polynucleotide. Nucleic acids contain various modified nucleotides.

### Identity

“Identity” describes the correlation between two nucleotide sequences in the present application. The calculation of identity of two aptamer nucleotide sequences in the present application does not include N₁, N₁₉, N₂₀, N₂₁, N₃₂ in Sequence (a). As for the present application, identity of two aptamer nucleotide sequences is determined by using, for instance, Needle program, preferably Needleman-Wunsch Algorithm (Needleman and Wunsch, 1970, J.Mol.Biol. 48: 443-453) executed in 3.0.0 version or later, of EMBOSS software package (EMBOSS: The European Molecular Biology Open Software Suite, Rice etc., 2000, Trends in Genetics 16: 276-277). Optional parameters in use are gap penalty 10, gap extension penalty 0.5 and EBLOSUM62 substitution matrix (EMBOSS version of BLOSUM62). Output result marked by Needle as “longest identity” (obtained by using the “−nobrief” option) serves as the percentage identity, and is calculated in a way as follows:

(Identical residue×100)/(Alignment length-Total number of gaps in alignment).

For instance, the sequence of Pepper (C3A) in Table 1 of the present application is N₁CAAAUCGUGGCGUGUCGN₁₉-N₂₀-N₂₁ACUGGCGCCGN₃₂, and the sequence of Pepper (C3U) is N₁CUAAUCGUGGCGUGUCGN₁₉-N₂₀—N₂₁ACUGGCGCCGN₃₂, and, according to the definition of the present application, their identity alignment should not include the nucleotide bases of N₁, N₁₉-N₂₀-N₂₁ and N₃₂, so the alignment result of their sequence identity is 96.3% (the difference being one nucleotide).

### Fluorophore Molecules

The “fluorophore molecules” in the present application are also called as “fluorophore” or “fluorescence molecules”. “Fluorophore molecules” in the present application are a kind of fluorophore molecules that can be conditionally activated, and show a relatively low quantum yield in the absence of nucleic acid aptamers. In specific embodiments, when a fluorophore is not bound to specific aptamers, its quantum yield is lower than 0.1, preferably lower than 0.01, and optimally lower than 0.001; when the fluorophore is bound to specific aptamers, its quantum yield will be enhanced by more than two times, preferably by more than 10 times, and optimally by more than 100 times. Fluorophore molecules are preferably water-soluble, non-toxic to cells and easy to penetrate membranes. Fluorophore of the present application can preferably enter cytoplasm or pericytoplasm through membrane or cell wall by means of active transport or passive diffusion. In the embodiments of the present application, the fluorophore can penetrate outer and inner membranes of Gram-negative bacteria, cell walls and membranes of plant cells, cell walls and membranes of fungi, membranes of animal cells, and GI and endothelial membranes of living animals.

The nucleic acid aptamer molecules in the present application can specifically bind a fluorophore and significantly increase its fluorescence value under excitation light of specific wavelength. The expressions such as “improving fluorescence signals”, “fluorescence increase”, “enhancing fluorescence intensity”, “improving fluorescence intensity” in the present application refers to the increase of the quantum yield of the fluorophore or the migration (relative to emission peaks of fluorophore itself in ethanol or aqueous solution) of the maximum emission peak of fluorescence signals under the excitation light of appropriate wavelength, or an increase of molar extinction coefficient, or two or more of the above. In a preferable embodiment of the present application, the quantum yield is increased by at least two times; in another preferable embodiment of the present application, the quantum yield is increased by at least 5 to 10 times; in another more preferable embodiment of the present application, the quantum yield is increased by at least 20 to 50 times; in another more preferable embodiment of the present application, the quantum yield is increased by at least 100 to 200 times; in another more preferable embodiment of the present application, the quantum yield is increased by at least 500 to 1,000 times; in another more preferable embodiment of the present application, the quantum yield is increased by at least 1,000 to 10,000 times; in another more preferable embodiment of the present application, the quantum yield is increased by more than 10,000 times; the light source used for exciting the fluorophore to produce fluorescence signals can be any appropriate lighting device, such as LED lamp, incandescent lamp, fluorescent lamp and laser; excitation light can be either emitted directly from these devices or obtained indirectly by means of other fluorophores, such as donor fluorophores of FERT, or donor luminophors of BRET.

### Target Molecules

The target molecules of the present application can be any biomaterial or micromolecules, including but not limited to: proteins, nucleic acid (RNA or DNA), lipid molecules, carbohydrates, hormones, cytokines, chemokines, and metabolite metal ions and so on. Target molecules can be molecules associated with diseases or pathogen infection.

In the structure shown in FIG. 1, the inserted nucleotide sequence replaced the stem-loop structures of N₁₉, N₂₀, N₂₁ in FIG. 1 by means of the aptamer molecules in the present application, wherein the nucleotide sequence can specifically identify/bind target molecules. In the absence of target molecules, aptamer molecules do not or weakly bind fluorophore molecules, and thus cannot significantly improve the fluorescence of fluorophore molecules under excitation light of appropriate wavelength; in the presence of target molecules, the binding of target molecules and the nucleotide sequence will promote binding of the aptamer molecules and fluorophore molecules, and thus can significantly improve the fluorescence of fluorophore molecules under excitation light of appropriate wavelength, thereby realizing detection, imaging and quantitative analysis of target molecules.

Target molecules can also be whole cells or molecules expressed on the entire cell surface. Typical cells include but are not limited to cancer cells, bacterial cells, fungal cells and normal animal cells. The target molecules can also be virus particles. At present, many aptamers of the afore-mentioned target molecules have been identified, and can be integrated into the polyvalent nucleic acid aptamers of the present application. RNA aptamers that have been reported to bind target molecules include but are not limited to: T4 RNA polymerase aptamer, HIV reverse transcriptase aptamer, and phage R17 capsid protein aptamer.

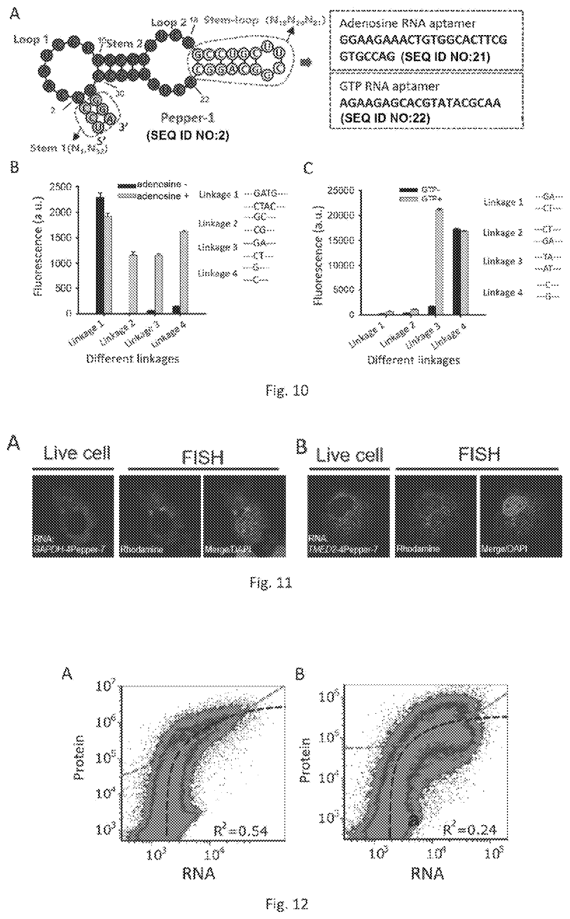

In a preferable embodiment of the present application, the target molecule is adenosine, and the corresponding probe sequence for identifying the target molecule is SEQ ID NO: 21 (as shown in FIG. 10A); in a preferable embodiment of the present application, the target molecule is GTP, and the corresponding probe sequence for identifying the target molecule is SEQ ID NO: 22 (as shown in FIG. 10A).

### Objective Nucleic Acid Molecules

“Objective nucleic acid molecules”, also called as “target nucleic acid molecules”, refer to the nucleic acid molecules to be detected, which can be either intracellular or extracellular; objective nucleic acid molecules include objective RNA molecules and objective DNA molecules. Objective nucleic acid molecules are connected with the nucleic acid aptamer molecules, and are bound to the nucleic acid aptamer molecules via fluorophore molecules so as to significantly improve the fluorescence value of fluorophore molecules under excitation light of appropriate wavelength, thereby detecting the content and distribution of objective nucleic acid molecules.

“Objective RNA molecules” in the present application include any RNA molecule, including but not limited to pre-mRNA, mRNA, pre-rRNA, rRNA, tRNA, hnRNA, snRNA, miRNA, siRNA, shRNA, sgRNA, crRNA, and long non-coding RNA of coding cells per se or exogenous expression products thereof, wherein phage capsid protein MCP identifies the binding sequence MS2RNA, phage capsid protein PCP identifies the binding sequence PP7RNA, λ phage transcription termination protein N identifies the binding sequence boxB RNA or the like. Target RNA can be fused at 5′ end or 3′ end or the position of N₁₉-N₂₀-N₂₁ of the RNA aptamer molecules of the present application.

“sgRNA” in the present application refers to single guide RNA (single guide RNA, sgRNA) formed by modifying tracrRNA and crRNA in the CRISPR/Cas9 system, wherein the sequence of about 20 nt at the 5′ end of the system targets DNA site via base pair complementation, and promotes the Cas9 protein to induce DNA double-strand break at this site.

### Concatemers of Nucleic Acid Aptamer

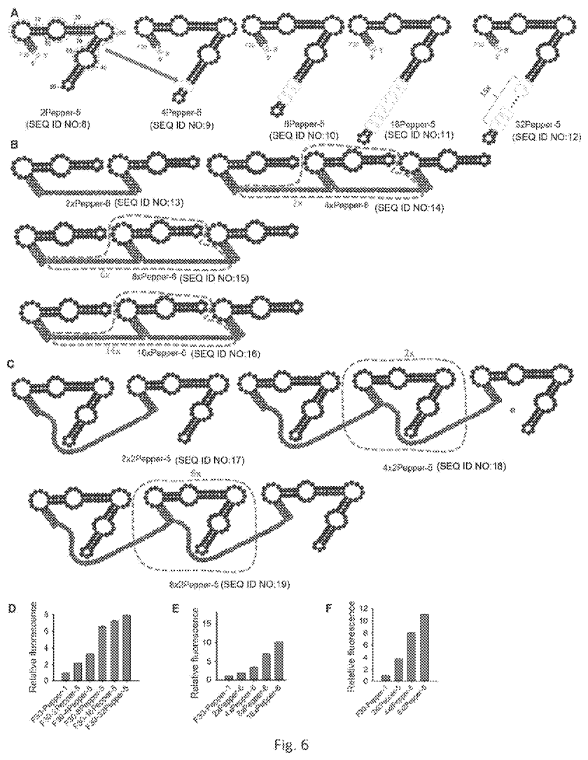

The nucleic acid aptamer molecules of the present application may further include concatemers that can bind multiple fluorophore molecules. The concatemers are connected by spacer sequences of appropriate length, and the number of Pepper structures in series may be 2, 3, 4, 5, 6, 7, 8, 9, 10 or greater. The concatermers may be in many forms. In a preferable embodiment of the present application, the series form is “Series 1”, as shown in FIG. 6A, and a preferable nucleotide sequence is SEQ ID NO: 8, 9, 10, 11 or 12, wherein 2Pepper-5 indicates Concatemer 1 with 2 Pepper-5 structures; in another preferable embodiment of the present application, the series form is “Series 2”, as shown in FIG. 6B, and a preferable nucleotide sequence is SEQ ID NO: 13, 14, 15 or 16, wherein 2×Pepper-6 indicates Concatemer 2 with 2 Pepper-6 structures; in another preferable embodiment of the present application, the series form is “Series 3”, as shown in FIG. 6C, and a preferable nucleotide sequence is SEQ ID NO: 17, 18 or 19, wherein 2×2Pepper-5 indicates Concatemer 3 with 4 Pepper-5 structures; in any form, the spacer sequences between the concatemers can be changed.

The monomer aptamers of the present application refer to aptamers containing only one Pepper structure, that is, the aptamers containing two stem structures, two loop structures and one stem-loop structure (FIG. 1A).

The polymer aptamers refer to aptamers containing more than one Pepper structure, including but not limited to the aptamer composed of several series forms as shown in FIG. 6.

### Aptamer-Fluorophore Complex

The aptamer-fluorophore complex of the present application contains one nucleic acid aptamer molecule and one or more fluorophore molecules. In an embodiment of the present application, the molecule complex containing one nucleic acid molecule and one fluorophore molecule is F30-Pepper-2-III-3, F30-Pepper-2-III-7, F30-Pepper-2-III-6, F30-Pepper-2-III-8, F30-Pepper-2-III-4, F30-Pepper-2-III-15, F30-Pepper-2-III-18 and F30-Pepper-2-III-21.

In another embodiment of the present application, nucleic acid molecules of the concatemer and a plurality of fluorophore molecules forms a complex, for instance, a complex 8Pepper-5-8x(III-3) formed in the way of “Series 1” by F30-8Pepper-5 containing 8 aptamer units and 8 fluorophore molecules, namely, III-3, 8Pepper-5-8x(III-7), 8Pepper-5-8x(III-6), 8Pepper-5-8x(III-8), 8Pepper-5-8x(III-4), 8Pepper-5-8x(III-15), 8Pepper-5-8x(III-18) and 8Pepper-5-8x(III-21). The molecule complexes may exist in vitro in the form of two separate solutions, or in the same solution, or in cells.

### Nucleic Acid Aptamer Function

The aptamer function of the present application means to significantly enhance fluorescence intensity of fluorophore molecules under excitation light of appropriate wavelength, and aptamers can be detected by function detection of nucleic acid aptamer as shown in common Experimental Method (V) in the embodiments. In a preferable embodiment of the present application, the fluorescence intensity is increased by at least two times (the fluorescence intensity is detected according to the Experimental Method (V)); in another preferable embodiment of the present application, the fluorescence intensity is increased by at least 5 to 10 times; in another more preferable embodiment of the present application, the fluorescence intensity is increased by at least 20 to 50 times; in another more preferable embodiment of the present application, the fluorescence intensity is increased by at least 100 to 200 times; in another more preferable embodiment of the present application, the fluorescence intensity is increased by at least 500 to 1,000 times; in another more preferable embodiment of the present application, the fluorescence intensity is increased by at least 1,000 to 10,000 times; in another more preferable embodiment of the present application, the fluorescence intensity is increased by more than 10,000 times.

### Secondary Structure of Nucleic Acid Aptamers

In the present application, the secondary structure of nucleic acid aptamers is obtained by simulation and prediction using mFold online analysis software (http://unafold.rna.albany.edu/?q=mfold). The stem structure in the secondary structure refers to a local double-strand structure formed by complementary pairing of hydrogen bonds in some regions of the single strand of nucleic acid aptamer molecules. In general, the formation of the double-strand structure does not require complementary pairing of all nucleotides in this region; in general, the stem structure will be formed when complementary pairing occurs between at least 50% of the nucleotides in a fragment of sequences N₁ and N₃₂, as well as N₁₉ and N₂₁ and the other fragment. If N₁ and N₃₂ are single nucleotides, the stem structure can be formed with complete complement of N₁ and N₃₂ (as shown in FIG. 1).

### DNA Molecules Expressing Nucleic Acid Aptamers

The DNA molecules contain a DNA sequence which can encode the nucleic acid aptamer molecules of the present application. The DNA molecules contain a nucleotide sequence R₁CCAATCGTGGCGTGTCGR₁₉-R₂₀-R₂₁ACTGGCGCCGN₃₂, as well as a nucleotide sequence with identity of at least 70%, wherein R₁ encodes N₁ in the General Formula Pepper structure, R₁₉ encodes N₁₉ in the General Formula Pepper structure, R₂₀ encodes N₂₀ in the General Formula Pepper structure, R₂₁ encodes N₂₁ in the General Formula Pepper structure, and R₃₂ encodes N₃₂ in the General Formula Pepper structure. The DNA molecules may also contain a promoter which controls DNA transcription, wherein the promoter is in operable connection to the DNA sequence encoding the nucleic acid aptamer. In an embodiment of the present application, the DNA molecule contains an U6 promoter; in another embodiment of the present application, the DNA molecules contain a CMV promoter. The DNA molecules may further contain a DNA sequence which encodes any objective nucleic acid molecule. In an embodiment of the present application, the DNA molecules encoding the objective RNA contain a DNA sequence (sequences for embedding RNA are respectively SEQ ID No: 24, 25) encoding glyceraldehyde-3-phosphate dehydrogenase (GAPDH) and transmembrane emp24 domain-containing protein 2 (TMED2). In another embodiment of the present application, the DNA molecules encoding the objective RNA contain a DNA sequence (sequences for embedding RNA are respectively SEQ ID No: 26, 27) encoding mCherry and TagBFP.

### Promoters

“Promoters” in the present application include promoters of eukaryotic and prokaryotic cells. Promoter sequences of eukaryotic cells are totally different from those of prokaryotic cells. Generally, eukaryotic promoters cannot be identified by RNA polymerases in prokaryotic cells or mediate RNA transcription. Similarly, prokaryotic promoters cannot be identified by RNA polymerases in eukaryotic cells or mediate RNA transcription either. The strength of different promoters varies greatly (strength refers to the ability to mediate transcription). According to actual application, strong promoters can be used for achieving high level transcription. For instance, high level expression is better for labeling, and, for evaluation of transcription behavior, lower level transcription will allow cells to process transcription in a timely manner. One or more suitable promoters can be selected for different host cells. For instance, being used in Escherichia coli cells, T7 phage promoter, lac promoter, trp promoter, recA promoter, ribosome RNA promoter, PR and PL promoters in λ phage, and other promoters, but not limited to: lacUV5 promoter, ompF promoter, bla promoter, lpp promoter etc. Moreover, a hybrid trp-lacUV5 promoter (tac promoter) or other Escherichia coli cells obtained through recombinant or synthetic DNA technology can all be used for transcribing the RNA aptamers of the present application. Some of the operator sequences in bacteria per se can combine with promoter sequences to form inducible promoters, and specific inducers need to be added at this moment to induce transcription of DNA molecules. For instance, the expression of lac operator needs to be induced by the addition of lactose or lactose analogues (IPTG), other operators including trp, pro or the like.

As mentioned above, the regulating sequence of 5′ end of DNA molecule decoding sequence is promoters. Suitable promoters need to be selected according to the promoter intensity either to obtain RNA apatmers via in vitro transcription or to express aptamers in cultured cells or tissues. Since the expression of aptamers in vivo can be genetically manipulated, another type of promoters is inducible promoters that induce DNA transcription in response to a specific environment, such as in a specific tissue, at a specific time, and in a specific developmental stage. These different promoters can be identified by RNA polymerase I, II or III.

Promotion of transcription in eukaryotic cells also needs suitable promoters, including but not limited to β-globulin promoter, CAG promoter, GAPDH promoter, β-actin promoter, actin promoter, Cstf2t promoter, SV40 promoter, PGK promoter, MMTV promoter, adenovirus Ela promoter, CMV promoter and so on. Termination of transcription in eukaryotic cells depends on the specific cleavage site in RNA sequence. Similarly, since the transcription genes of RNA polymerase are different, transcriptional terminators also vary significantly. However, those skilled in the art can realize screening of suitable 3′ transcriptional terminator sub-regions by means of routine experimental skills.

### Expression System

The “expression system” of the present application, also called as “expression vector”, contains and is integrated with DNA molecules expressing nucleic acid aptamer. The expression system of the present application can be a plasmid or a virus particle.

Recombinant virus of “expression vector” can be obtained by transfection of plasmids into viral-infected cells. Suitable vectors include but are not limited to virus vectors such as λ vector system gt11, gt WES.tB, Charon 4, and plasmid vectors include pBR322, pBR325, pACYC177, pACYC184, pUC8, pUC9, pUC18, pUC19, pLG399, pR290, pKC37, pKC101, pBluescript II SK+/− or KS+/− (see Stratagene cloning system), pET28 series, pACYCDuet1, pCDFDuet1, pRSET series, pBAD series, pQE, pIH821, pGEX, pIIIEx426 RPR and so on.

A large number of host expression systems can be used for expressing the DNA molecules of the present application. Mostly, the vector system has to be compatible to the host cells in use, wherein the host vector system includes but is not limited to: transformed phage DNA, or plasmid DNA, or bacteria with cosmid DNA; yeast containing yeast vector; mammalian cells infected with a virus (e.g. adenovirus, adeno-associated virus, retrovirus); insect cells infected with a virus (e.g. baculovirus); and plant cells infected with bacteria or transformed by means of particle bombardment. Expression elements in the vectors are significantly different in strength and characteristics. Any one or more suitable transcription elements can be selected according to the host-vector system in use.

Once the constructed DNA molecules are cloned into the vector system, it will be easy to transfer them into host cells. Based on different vector or host cell systems, the method includes but is not limited to transformation, transduction, conjugation, fixation, electrical transfer or the like.

An embodiment of the present application provides expression plasmids pET28a-T7-F30-Pepper-2, pLKO.1-F30-Pepper-2 and pYES2.1-F30-Pepper-2 containing DNA molecules for encoding F30-Pepper-2 RNA. Another embodiment of the present application provides expression plasmid pLKO.1-F30-8Pepper-5 containing DNA molecules for encoding F30-8Pepper-5 RNA. Another embodiment of the present application provides expression plasmids pCDNA3.1 hygro(+)-BFP-4Pepper-7, pCDNA3.1 hygro(+)-mCherry-4Pepper-7, pCDNA3.1 hygro(+)-GAPDH-4Pepper-7 and pCDNA3.1 hygro(+)-TMED2-4Pepper-7 containing DNA molecules for encoding BFP-4Pepper-7, mCherry-4Pepper-7, GAPDH-4Pepper-7 and TMED2-4Pepper-7. Another embodiment of the present application provides expression plasmids psgRNA-Pepper-8 (loop 1), psgRNA-Pepper-8 (tetraloop) and psgRNA-Pepper-8 (loop 1 and tetraloop) containing DNA molecules for encoding sgRNA-Pepper-8 (loop 1), sgRNA-Pepper-8 (tetraloop), sgRNA-Pepper-8 (loop 1 and tetraloop). Another embodiment of the present application provides an expression plasmid pLKO.1-4Pepper-9-MS2 containing DNA molecules for encoding 4Pepper-9-MS2.

The present application further provides expression vectors integrated with DNA molecules for encoding nucleic acid aptamers, but with vacant encoding DNA sequences of objective RNA molecules, wherein the vacancy of encoding DNA sequences of objective RNA molecules allows the users to choose DNA sequences of objective RNA molecules to be detected, for instance, corresponding encoding DNA sequence of GAPDH mRNA inserts the DNA sequence into the expression vector of the present application by means of standard recombination DNA technology, and guides the obtained expression vector into the host cells of (transfection, transform, infection and so on), thereby detecting the content and distribution of objective RNA.

### Host Cells

“Host cells” in the present application include but are not limited to bacteria, yeast, mammalian cells, insect cells, plant cells, zebra fish cells, fruit fly cells, and nematode cells. Host cells preferably are cultured cells in vitro or whole in vivo living tissue. Mammalian cells contained in the host cells of the present application include but are not limited to 297T, COS-7, BHK, CHO, HEK293, HeLa, H1299, stem cells of fertilized eggs, inducible totipotent stem cell, and primary cells isolated directly from mammalian tissues and so on; Escherichia coli cells contained therein include but are not limited to BL21 (DE3), BL21 (DE3, Star), TOP10, Mach1, and DH5α; and yeast cells contained therein include but are not limited to BY4741, BY4742, and AH109.

### Detection Array

The detection array of the present application includes one or more nucleic acid aptamer molecules of the present application, wherein the nucleic acid aptamer molecules are anchored at discrete locations on the array surface composed of solid supports, including but not limited to glass, metals, and ceramic and so on. The nucleic acid aptamer molecules of the present application can be anchored to the array surface by, but not limited to, the following methods: (1) labeling the 5′ end or 3′ end of the nucleic acid aptamer molecule with biotin, coating the array surface with streptavidin, and anchoring the nucleic acid aptamer molecule by specific binding of biotin and streptavidin; (2) identifying the binding sequence MS2 by using the phage capsid protein MCP, identifying the biding sequence PP7 by using the phage capsid protein PCP or identifying the binding sequence boxB by using the X phase transcription terminating protein N, fusing the RNA sequence at the 5′, 3′ or stem-loop structure of the nucleic acid aptamer molecules, coating the array surface with protein MCP, PP7 or λ_{N} protein identified and bound thereby, and anchoring the nucleic acid aptamer molecules through the specific effects of MS2 with MCP protein, PP7 with PCP protein or boxB RNA with λ_{N} protein; (3) fusing a fragment of RNA or DNA sequence at the 5′ end or 3′ end of the nucleic acid aptamer molecules, anchoring an RNA sequence in complementary pairing with the RNA sequence segment or an DNA sequence in complementary pairing with the DNA sequence segment on the array surface, and anchoring the nucleic acid aptamer molecules on the array surface by means of the molecular hybridization principle. The detection array can be used for detecting the presence or absence of the target molecule as well as the concentration level, as a result, the nucleic acid aptamer molecules be bound with the fluorophore molecules and significantly improve the fluorescence intensity under excitation light of appropriate wavelength only with the presence of target molecules; and, within a certain range, the higher the concentration of the target molecules, the higher the fluorescence intensity.

### Kit

Kit of the present application includes the nucleic acid aptamer molecules and/or the fluorophore molecules of the present application, and corresponding instructions; or includes an expression system for expressing the nucleic acid aptamer molecules and/or the fluorophore molecules, and corresponding instructions; or includes host cells expressing the aptamer molecular expression system and/or the fluorophore molecules, and corresponding instructions. The nucleic acid aptamer molecules and the fluorophore molecules in the kits respectively exist in individual solutions, or exist in the same solution.

The present application will be further elaborated in the following examples, which are merely used for giving examples, rather than limiting the scope of the present application. The examples mainly adopt conventional cloning methods of molecular biology in genetic engineering, which are well known to ordinary technicians in this field, for instance, relevant chapters from Lab Ref: A Handbook of Recipes, Reagents, and Other Reference Tools for Use at the Bench by Jane Roskams et al, and Molecular Cloning—A Laboratory Manual (Third Edition, August 2002, Science Press, Beijing) written by Sambrook. J, D. W. Russell and translated by Peitang HUANG et al. Based on the following examples, it easy for one skilled in the art to successfully implement the present application after slight amendment and change made according to actual situations.

In the examples, the pCDNA3.1 hygro (+) plasmid vector was purchased from Invitrogen Company, pLKO.1-puro plasmid vector was purchased from Sigma Company, pET28a plasmid vector was purchased from Novagen Company, pYES2.1 TOPO TA plasmid vector was purchased from Invitrogen. All primers used for PCR were synthesized, purified and identified correct via mass spectrometry by Shanghai Generay Biotech Co., Ltd. Expression plasmids constructed in the examples all went through the sequence determination accomplished by JIE LI BIOLOGY. Taq DNA polymerase used in all examples was purchased from Yeasen Biotechnology (Shanghai) Co., Ltd., PrimeSTAR DNA polymerase was purchased from TaKaRa Company, and corresponding polymerase buffers and dNTP were included during purchasing of these three kinds of polymerases. EcoRI, BamHI, BglII, HindIII, NdeI, XhoI, SacI, XbaI, SpeI and other restriction endonuclease, T4 ligase, T4 phosphorylase (T4 PNK), and T7 RNA polymerase were purchased from Fermentas company, and corresponding polymerase buffers and so on were included during purchasing. Hieff Clone™ One Step cloning kits used in the examples were purchased from Yeasen Biotechnology (Shanghai) Co., Ltd. Unless otherwise stated, chemical reagents such as inorganic salts were all purchased from Shanghai Chemical Reagent Company of Sinopharm. Kanamycin was purchased from Ameresco Company; Amp was purchased from Ameresco Company; and 384-well and 96-well fluorescence detection blackboard was purchased from Grenier Company. DFHBI-1T and DFHO were purchased form Lucerna Company. GTP and SAM were purchased from Sigma Company.

DNA purification kits used in the examples were purchased from BBI Company (Canada), ordinary plasmid micro extraction kits were purchased from Tiangen Biotech (Beijing) Co., Ltd. BL21 (DE3, Star) bacterial strains were purchased from Invitrogen Company. 293T/17 cells and COS-7 cells were purchased from Cell Bank of Committee of Typical Culture Collection, Chinese Academy of Sciences. BY4741 yeast strain was purchased from Shanghai Weidi Biotechnology Co., Ltd.

Main instruments used in the examples: Synergy Neo2 Multiscan Spectrum (Bio-Tek Company of America), X-15R high speed freezing centrifuge (Beckman Company of America), Microfuge22R tabletop high speed freezing centrifuge (Beckman Company of America), PCR amplifier (Biometra Company of German), in vivo imaging system (Kodak Company of America), photometer (Wako Company of Japan), nucleic acid electrophoresis apparatus (Shenneng Bocai Company).

Meanings of abbreviations are as follows: “h” refers to hours, “min” refers to minutes, “s” refers to seconds, “d” refers to days, “μL” refers to micro-liters, “ml” refers to milliliters, “L” refers to liters, “bp” refers to base pairs, “mM” refers to millimoles, and “μM” refers to micromoles.

### Commonly Used Experimental Methods and Materials in the Examples

### 1. Preparation of Nucleic Acid Aptamer

The cDNA corresponding to the RNA to be detected was amplified using primers containing T7 promoter, and the RNA was transcribed using T7 RNA polymerase (purchased from Fermentas) using the double-stranded cDNA as the template. 10 μL 3 M NaAc, 115 μL DEPC water were added into the 20 μL transcription system and mixed well. Then 150 μL phenol-chloroform-isopropanol mixture (phenol:chloroform:isopropanol=25:24:1) was added and shaken to mix well. The mixture was centrifugated at 10,000 rpm for 5 min, and the supernatant was taken. An equal volume of chloroform solution was added into the supernatant, shaken, mixed, centrifugated at 10,000 rpm for 5 min. The supernatant was taken and the same procedure was repeated once again. 2.5 times the volume of absolute ethanol was added into the supernatant and mixed. The mixture was placed in a refrigerator at −20 °C for 30 min, and centrifugated at 12000 rpm at 4 °C for 5 min. The supernatant was discarded and the precipitate was washed twice with pre-cooled 75% ethanol. After the ethanol was evaporated, an appropriate amount of screening buffer was added to resuspend the pellet. The solution was treated at 75 °C for 5 minutes and placed at room temperature for more than 10 minutes for subsequent experiments.

### 2. Cell Culture and Transfection

All the cells in this example were cultured in DMEM (high glucose) supplemented with 10% FBS, streptomycin and penicillin in a CO2 incubator. All cell lines were split at a confluence of 80-90%. The FuGENE®HD (purchased from Promega) was used for transfection according to the instructions.

### 3. Fluorescence Imaging

The main imaging experiments in the examples were carried out using a Leica SP8 confocal laser scanning microscope equipped with a HCXPL APO 63.0×1.47 OIL objective, and a HyD detector. A 488 nm laser was used to image the fluorescence of Pepper-III-3 complex. A 405 nm and a 561 nm laser were used to image the fluorescence of BFP and mCherry, respectively. A 458 nm laser, a 458 nm laser, a 488 nm laser, a 488 nm laser, a 488 nm laser, a 561 nm laser and a 561 nm laser were used to image the fluorescence of Pepper-III-7, Pepper-III-6, Pepper-III-8, Pepper-III-4, Pepper-III-15, Pepper-III-18 and Pepper-III-21, respectively. A 448 nm laser was used to image the fluorescence of Broccoli-DFHBI-1T and Corn-DFHO.

### 4. Homologous Recombination-Based Construction of Recombinant Plasmids

(1) Preparation of linearized vector: select a suitable cloning site and linearize the vector by restriction enzyme-based digestion or inverse PCR amplification.
(2) PCR amplification of the insertion fragments: use the forward and reverse PCR primers containing 15-25 bp (excluding restriction site) homologous sequence of the linearized vector to amplify the template to obtain insertion fragment harboring the identical sequences corresponding to the two ends of the linearized vector.
(3) Determination of the concentrations of linearized vector and insertion fragment: dilute the linearized vector and insertion fragment, take 1 μL of the original solution and diluted solution to perform agarose gel electrophoresis, determine the concentration of the fragments by comparing the intensities of the bands to those of the DNA molecular weight standard (DNA maker).
(4) Recombination reaction: the optimal amount of vector used in the recombination reaction system is 0.03 pmol; the optimal molar ratio of linearized vector to insertion fragment is 1:2-1:3, i.e., the optimal amount of insertion fragment is 0.06-0.09 pmol.

| Component | Recombination reaction | |
|---|---|---|
| ddH₂O | Maximum to 20 μL | |
| 2 × Hieff Clone Enzyme Premix | 10 | μL |
| (from Yeasen) | | |
| linearized vector | X | μL |
| insertion fragment | Y | μL |

X and Y are calculated according to the formula to obtain the linearized vector and insert fragment. After the preparation of the system, mix the components and place at 50 °C for 20 min. When the insertion fragment >5 kb, the incubation temperature can be extended to 25 min. After the reaction is complete, it is recommended to cool the reaction tube on ice for 5 min. The reaction product can be transformed directly, or stored at −20 °C before transformation needed.

### 5. Characterization of the Aptamers

Preparation of Pepper or Pepper mutant aptamer according to the commonly used experimental method (1). 5 μM aptamer and 1 μM fluorophore were incubated in the detection buffer (40 mM HEPES, pH 7.4, 125 mM KCl, 5 mM MgCl2, 5% DMSO). The maximal excitation and emission peaks of the aptamer-fluorophore complexes were determined using a Synergy Neo2 multifunctional microplate reader. The fluorescence intensities of the aptamer-fluorophore complexes at the maximal excitation and emission peaks were determined using a Synergy Neo2 multifunctional microplate reader. The control sample (1 μM fluorophore without aptamer) was also measured under the same conditions. Then the ratios of fluorescence intensity were calculated. For example, the fluorescence maximal excitation and emission peaks of the complex of 5 μM F30-Pepper-2 and 1 μM III-3 fluorophore are 485 nm and 530 nm, respectively. The fluorescence intensity of the complex at 485±10 nm excitation and 530 nm±10 nm emission was 36,000, the fluorescence intensity of the control sample (1 μM III-3 fluorophore only) under the same detection conditions was 10. Thus, the activation ratio of F30-Pepper-2 aptamer on III-3 fluorophore was 3,600-fold.

### Example 1. The Secondary Structure of Pepper Aptamer

The secondary structure of Pepper aptamer was analyzed using the mFold online RNA structure analysis software. Pepper contains two stems, two loops and one stem-loop structures (FIG. 1A). For one of the stem 1 and stem-loop, the predicted secondary structure of Pepper-1 (SEQ ID NO: 1) was shown in FIG. 1B.

### Example 2. Characterization of Pepper-III-3 Complex

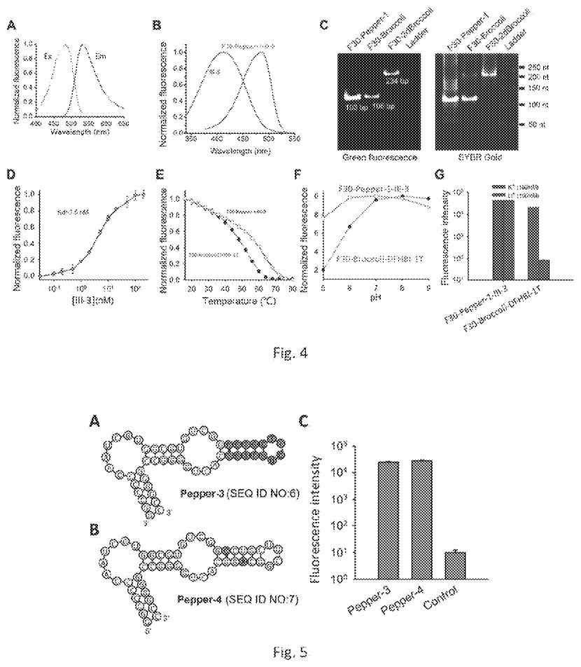

In order to detect the spectral properties of the Pepper-III-3 complex, F30-Pepper-1 (SEQ ID NO: 2) RNA was prepared according to the commonly used experimental method (1). 1 μM III-3 with 5 μM F30-Pepper-1 was incubated. The results showed that the maximum excitation and emission of the F30-Pepper-1-III-3 complex were 485 nm, and 530 nm, respectively (FIG. 4A). In order to detect the absorption difference between F30-Pepper-1-III-3 complex and III-3 fluorophore itself, the absorption of F30-Pepper-1 and III-3 complex (5 μM III-3 and 25 μM F30-Pepper-1) or 5 μM alone III-3 was detected. The results showed that the maximum absorption of F30-Pepper-1-III-3 complex has a large red shift relative to III-3 itself, and the maximum light absorption is 484 nm (FIG. 4B).

In order to detect whether Pepper binds to III-3 in the form of monomer or polymer, F30-Pepper-1 was identified by the Native PAGE using the monomeric F30-Broccoli (SEQ ID NO: 4) and F30-2dBroccoli (SEQ ID NO: 5) (Filonov et al. Journal of the American Chemical Society 2014. 136: 16299-16308; Filonov et al. Chemistry & biology 2015. 22: 649-660) as the controls. The fluorescence imaging result was compared with the staining result by SYBR Gold (universal nucleic acid dye that was purchased from Invitrogen). The results showed that F30-Pepper-1 was located at about 100 bp similar to F30-Broccoli, which was consistent with its actual size of 103 bp. Therefore, the results indicate that F30-Pepper-1 binds to III-3 in the form of monomer (FIG. 4C).

In order to detect the binding constant of Pepper and III-3, 2 nM F30-Pepper-1 was incubated with different concentrations of III-3 and their fluorescence was determined. The results showed that the binding constant of F30-Pepper-1 and III-3 was 3.5 nM (FIG. 4D).

In order to test the thermostability of Pepper, 10 μM III-3 was incubated with 1 μM F30-Pepper-1, and then was placed at different temperatures for 5 minutes before the fluorescence was determined. 10 μM DFHBI-1T was incubated with 1 μM F30-Broccoli to be as the control. The results showed that the Tₘ of F30-Pepper-1 is 55 °C, which is significantly higher than the 48 °C of F30-Broccoli (FIG. 4E), indicating that F30-Pepper-1 has better thermostability.

In order to test the stability of Pepper-III-3 complex at different pH, F30-Pepper-1-III-3 complex was incubated in different pH environment for 60 min before the fluorescence was detected. The F30-Broccoli-DFHBI-1T complex was used as the control. The results showed that the F30-Pepper-1-III-3 complex maintained a high fluorescence in the range of pH 5-9, while the fluorescence of F30-Broccoli-DFHBI-1T decreased rapidly with the decrease of pH (FIG. 4F), indicating that the F30-Pepper-1-III-3 complex has better pH stability.

In order to detect the dependence of Pepper-III-3 complex on K⁺, 1 μM F30-Pepper-1 and 5 μM III-3 were incubated in buffer containing 100 mM KCl or 100 mM LiCl, respectively. The solution was treated at 70 °C5 for 5 min and placed at room temperature for more than 15 min before the fluorescence was detected. The F30-Broccoli-DFHBI-1T complex was used as the control. Previous studies have shown that the structure of Broccoli contains a G-quadruplex, and the stability of the G-quadruplex structure is highly dependent on K⁺, which was consistent with the results. The fluorescence of F30-Broccoli-DFHBI-1T complex in LiCl buffer was a few percent of that in KCl buffer (FIG. 4G). In comparison, the fluorescence of the F30-Pepper-1-111-3 complex did not depend on K⁺ (FIG. 4G), indicating that Pepper does not contain G-quadruplex on its structure.

### Example 3. Fluorescence Activation of III-3 Fluorophore by Different Pepper Mutants

In order to detect the fluorescence activation of III-3 fluorophore by different Pepper mutant, the Pepper-1 sequence in F30-Pepper-1 was mutated according to Table 1. The Pepper RNA containing different mutations were prepared according to the commonly used experimental method (1). 1 μM III-3 was incubated with 5 μM RNA and their fluorescence activation folds were determined according to the commonly used experimental method (5). The results showed that most of the F30-Pepper-1 mutants containing single mutation retained a strong fluorescence activation of III-3 (>10 times) (Table 2). several F30-Pepper-1 mutants containing 2-7 mutations still retained strong fluorescence activation of III-3 (>100 times) (Table 3). In summary, many Pepper mutants containing single and multiple mutants still retain the ability to activate the fluorescence of III-3 fluorophore.

TABLE 2
Activation of III-3 by Pepper mutants with single mutation

| | Activation | | Activation | | Activation |
|---|---|---|---|---|---|
| Mutant | folds | Mutant | folds | Mutant | folds |
| F30-Pepper-1 | 3600 | G10A | 847 | G18C | 1028 |
| C3G | 360 | G10C | 856 | A22U | 87 |
| C3A | 2484 | G11U | 1512 | A22G | 687 |
| C3U | 2016 | G11A | 1526 | A22C | 147 |
| A4U | 1836 | G11C | 325 | C23G | 65 |
| A4G | 2160 | C12G | 2125 | C23A | 547 |
| A4C | 2772 | C12A | 458 | G26U | 532 |
| A5G | 1800 | C12U | 2268 | C27G | 1875 |
| A5C | 2628 | G13U | 587 | C27A | 186 |
| U6A | 1872 | G13A | 792 | C27U | 3158 |
| U6G | 1980 | G13C | 1758 | G28U | 873 |
| U6C | 2088 | U14A | 1524 | G28A | 42 |
| C7A | 1044 | U14G | 3152 | G28C | 145 |
| C7U | 2268 | G15C | 15 | C29G | 2145 |
| G8C | 3168 | U16A | 28 | C29U | 1437 |
| G8A | 324 | U16G | 125 | C29A | 18 |
| U9A | 2124 | C17A | 52 | C30G | 145 |
| U9C | 72 | C17U | 1268 | C30U | 2587 |
| G10U | 900 | G18U | 1024 | C30A | 1596 |

Note:
F30-Pepper-1 in Table 2 is an aptamer with the sequence of SEQ ID NO: 2. Other aptamers are generated by mutating the corresponding nucleotide in FIG. 1A within the Pepper-1 sequence in F30-Pepper-1.

TABLE 3
Activation of III-3 by Pepper mutants with multiple mutations

| Mutant | Activation folds | Mutant | Activation folds | Mutant | Activation folds |
|---|---|---|---|---|---|
| F30-Pepper-1 | 3600 | U9A/C27U | 478 | C17U/C29U | 125 |
| C2G/G31C | 3124 | U9A/C27G | 178 | C17U/C30U | 578 |
| C2U/G31A | 3256 | U9A/C27A | 17 | C17U/C30A | 268 |
| C2A/G31U | 3365 | G13C/C17U | 87 | C17U/C27U | 689 |
| G10A/C30U | 3147 | U14A/C17U | 256 | C17U/C27G | 876 |
| G10C/C30G | 3514 | U14G/C17U | 145 | G18U/C29G | 148 |
| G10U/C30A | 3254 | G11U/C17U | 478 | G18U/C27G | 547 |
| A5G/U6C | 500 | C12G/C30U | 445 | G18U/C27U | 789 |
| A5C/C7U | 368 | C12A/C29G | 14 | G18U/C30U | 698 |
| U6A/C27G | 457 | G11A/C29U | 456 | G18U/C30A | 789 |
| U6G/C27U | 880 | G11C/C29U | 15 | A22G/C30U | 157 |
| U6C/C27U | 758 | C12U/C30U | 568 | C23A/C29G | 78 |
| C7A/C29G | 245 | G13U/C17U | 125 | G26U/C27U | 89 |
| C7U/C29G | 356 | G13C/C17U | 87 | C27G/C29G | 1025 |
| G8C/C29G | 268 | U14A/C17U | 256 | C27G/C30U | 1278 |
| U9A/C30U | 248 | U13G/C16U | 145 | C27G/C30A | 785 |
| U9A/C30A | 356 | C17U/C29G | 457 | C27U/C29G | 1457 |
| C27U/C30U | 786 | C29G/C30U | 1203 | C29U/C30A | 536 |
| G28U/C29G | 99 | C29G/C30A | 986 | U6C/G8C/C2 | 256 |
| G28U/C30U | 125 | C29U/C30U | 487 | U9A/U14G/C | 258 |
| C2G/G31C/ | 256 | C21U/C30U/ | 324 | C2G/G31C/ | 652 |
| A5G/U6C | | C17U/C27G | | G10U/C30A/ | |
| | | | | U6G/C27U | |
| G10A/C30U/ | 68 | C2U/G31A/ | 126 | C2U/G31A/ | 458 |
| C17U/C27U | | C27G/C29G | | G10A/C30U/ | |
| | | | | U6G/C27U | |
| G10A/C30U/ | 126 | C2U/G31A/ | 245 | C2U/G31A/ | 796 |
| C17U/C27U | | C12U/C30U | | G10C/C30G/ | |
| | | | | U6G/C27U | |
| G10C/C30G/ | 234 | C2A/G31U/ | 158 | C2U/G31A/ | 564 |
| U9A/C27U | | U6G/C27U | | G10U/C30A/ | |
| | | | | U6G/C27U | |
| G10C/C30G/ | 257 | C2A/G31U/ | 59 | C2A/G31U/ | 358 |
| U6G/C27U | | C12U/C30U | | G10A/C30U/ | |
| | | | | U6G/C27U | |
| G10U/C30A/ | 156 | C2U/G31A/ | 2583 | C2A/G31U/ | 498 |
| C17U/C27U | | G10U/C30A | | G10C/C30G/ | |
| | | | | U6G/C27U | |
| G10U/C30A/ | 87 | C2A/G31U/ | 3105 | C2A/G31U/ | 786 |
| U6G/C27U | | G10A/C30U | | G10U/C30A/ | |
| | | | | U6G/C27U | |
| C2G/G31C/ | 2365 | C2A/G31U/ | 3475 | C2G/G31C/ | 236 |
| G10A/C30U | | G10C/C30G | | G10A/C30U/ | |
| | | | | U9A/U14G/C | |
| C2G/G31C/ | 2684 | C2A/G31U/ | 3024 | C2G/G31C/ | 245 |
| G10C/C30G | | G10U/C30A | | G10C/C30G/ | |
| | | | | U9A/U14G/C | |
| C2G/G31C/ | 3125 | C2G/G31C/ | 758 | C2G/G31C/ | 198 |
| G10U/C30A | | G10A/C30U/ | | G10U/C30A/ | |
| | | U6G/C27U | | U9A/U14G/C | |
| C2U/G31A/ | 2578 | C2G/G31C/ | 736 | C2U/G31A/ | 178 |
| G10A/C30U | | G10C/C30G/ | | G10A/C30U/ | |
| | | U6G/C27U | | U9A/U14G/C | |
| C2U/G31A/ | 2986 | C2G/G31C/ | 652 | C2U/G31A/ | 196 |
| G10C/C30G | | G10U/C30A/ | | G10C/C30G/ | |
| | | U6G/C27U | | U9A/U14G/C | |
| C2A/G31U/ | 216 | C2A/G31U/ | 103 | C2A/G31U/ | 156 |
| G10A/C30U/ | | G10C/C30G/ | | G10U/C30A/ | |
| U9A/U14G/C | | U9A/U14G/C | | U9A/U14G/C | |

### Example 4. Base-Modified Pepper's Activating Effect on I-3

In order to detect the activation of III-3 by modified Pepper, base-modified Pepper-3 was synthesized (SEQ ID NO: 6, the underlined nucleotides in GGCCCCCAAUCGUGGCGUGUCGGCCUGCUUCGGCAGGCACUGGCGCCGG GGCC contain deoxyribonucleotide bases) and Pepper-4 (SEQ ID NO: 7, the underlined nucleotides in GCCCCCCAAUCGUGGCGUGUCGGCCUGCUUCGGCAGGCACUGGCGCCGG GGGCC contain 2′-F modification) (synthesized by Shanghai GenePharma Co., Ltd), which contain deoxyribonucleotides replacement (shaded bases in FIG. 5A) or 2′-F modification (shaded bases in FIG. 5B) in the stem-loop region of Pepper. Detection of the fluorescence activation of III-3 fluorophore by these modified Peppers was carried out according to the commonly used experimental method (5). The results showed that the modified Pepper-3 and Pepper-4 could still significantly activate the fluorescence of III-3 fluorophore (FIG. 5C).

### Example 5. Pepper Tandem Arrays

In order to detect the fluorescence activation of III-3 by different Pepper arrays, Pepper is connected to form tandem arrays in different forms, including the following three types:
(1) “tandem array 1” (FIG. 6A), different copies of Pepper are connected via “head-to-tail” to generate nPepper (where n represents any copy number of Pepper). In this example, the cDNA encoding F30-2Pepper-5, F30-4Pepper-5, F30-8Pepper-5, F30-16Pepper-5 and F30-32 Pepper-2 (The sequences of the RNA aptamer are SEQ ID NO: 8, SEQ ID NO: 9, SEQ ID NO: 10, SEQ ID NO: 11, SEQ ID NO: 12, respectively). After PCR amplification, the RNA aptamers were prepared according to the commonly used experimental methods (1). 0.1 μM RNA aptamer was incubated with 10 μM III-3 and fluorescence was measured according to the commonly used experimental method (5). The results showed that the fluorescence of nPepper-III-3 increased along with the increasing copy number of Pepper (n) (FIG. 6D). When n>8, with the increase of n, the fluorescence of nPepper-III-3 did not increase along with the increasing n, but was still much higher than that of Pepper-III-3 (FIG. 6D), indicating that the “tandem array 1” can be used to increase the fluorescence intensity of Pepper-III-3 complex.
(2) “tandem array 2” (FIG. 6B), Pepper serves as a structural unit and is connected to generate n×Pepper (where n represents any copy number of Pepper). In this example, the cDNAs encoding 2×Pepper-6, 4×Pepper-6, 8×Pepper-6 and 16×Pepper-6 (The sequences of the RNA aptamer are SEQ ID NO: 13, SEQ ID NO: 14, SEQ ID NO:15 and SEQ ID NO:16 respectively) were commercial synthesized. The RNA aptamers were prepared according to the commonly used experimental methods (1). 0.1 μM RNA aptamer was incubated with 10 μM III-3 and fluorescence was measured according to the commonly used experimental method (5). The results showed that that the fluorescence of n×Pepper-III-3 also increased along with the increasing copy number of Pepper (n) (FIG. 6E), indicating that the fluorescence intensity of Pepper-III-3 complex can be increased through the form of “tandem array 2”.
(3) “tandem array 3” (FIG. 6C), which is a combination of “tandem array 1“and” tandem array 2”. nPepper generated from “tandem array 1” serves as a structural unit and is connected to generate n1×n2Pepper according to the form of “tandem array 2” (where n1 and n2 represent any copy number of Pepper). In this example, the cDNAs encoding 2×2Pepper-5, 4×2Pepper-5, and 8×2Pepper-5 (The sequences of the RNA aptamer are SEQ ID NO: 17, SEQ ID NO: 18 and SEQ ID NO: 19, respectively) were commercial synthesized. The RNA aptamers were prepared according to the commonly used experimental methods (1). 0.1 μM RNA aptamer was incubated with 20 μM III-3 and fluorescence was measured according to the commonly used experimental method (5). The results showed that the fluorescence intensity of Pepper-III-3 obtained by the form of “tandem array 3” was significantly higher than that of Pepper-III-3 (FIG. 6F), indicating that the fluorescence of Pepper-III complex can be improved by the form of “tandem array 3”.

### Example 6. Characterization of III-3 Analogues

F30-Pepper-1 RNA aptamer was prepared according to the commonly used experimental methods (1), and was used to detect the properties of III-3 analogues upon Pepper binding, including the fluorescence spectrum, extinction coefficient, quantum yield, fluorescence activation fold and the binding constant (Kd). The results were shown in Table 4. From the data shown in the table, F30-Pepper-1 still could activate the fluorescence of III-3 analogues.

TABLE 4
The properties of F30-Pepper-1 RNA aptamer with different fluorophores

| | | | Extinction | | | |
|---|---|---|---|---|---|---|
| | Exₘₐₓ | Emₘₐₓ | coefficient | Quantum | Activation | K_{d} |
| | (nm) | (nm) | (M⁻¹ cm⁻¹) | yield | fold | (nM) |
| F30-Pepper-1-III-7 | 443 | 485 | 49100 | 0.42 | 691 | 8.0 |
| F30-Pepper-1-III-6 | 435 | 497 | 54700 | 0.57 | 16601 | 6.7 |
| F30-Pepper-1-III-8 | 458 | 508 | 42500 | 0.30 | 9091 | 27.0 |
| F30-Pepper-1-III-4 | 458 | 514 | 44100 | 0.45 | 4748 | 12.0 |
| F30-Pepper-1-III-15 | 491 | 525 | 74100 | 0.70 | 585 | 3.8 |
| F30-Pepper-1-III-3 | 485 | 530 | 65300 | 0.66 | 3595 | 3.5 |
| F30-Pepper-1-III-18 | 515 | 599 | 54400 | 0.43 | 708 | 18.0 |
| F30-Pepper-1-III-21 | 577 | 620 | 100000 | 0.58 | 12600 | 6.1 |
| F30-Pepper-1-III-1 | 482 | 528 | 59600 | 0.62 | 1256 | 6.3 |
| F30-Pepper-1-III-2 | 481 | 527 | 62300 | 0.52 | 1563 | 12.3 |
| F30-Pepper-1-III-5 | 462 | 501 | 23000 | 0.19 | 697 | 45.3 |
| F30-Pepper-1-III-9 | 455 | 502 | 39600 | 0.23 | 1360 | 23.2 |
| F30-Pepper-1-III-10 | 459 | 506 | 40200 | 0.26 | 2365 | 18.3 |
| F30-Pepper-1-III-11 | 453 | 492 | 42000 | 0.15 | 596 | 53.3 |
| F30-Pepper-1-III-12 | 452 | 486 | 39600 | 0.16 | 650 | 63.2 |
| F30-Pepper-1-III-13 | 476 | 512 | 52300 | 0.23 | 853 | 35.1 |
| F30-Pepper-1-III-14 | 465 | 512 | 45600 | 0.18 | 453 | 64.3 |
| F30-Pepper-1-III-16 | 492 | 534 | 78600 | 0.76 | 698 | 6.1 |
| F30-Pepper-1-III-17 | 508 | 596 | 52300 | 0.51 | 584 | 23.2 |
| F30-Pepper-1-III-19 | 498 | 586 | 42500 | 0.36 | 542 | 36.5 |
| F30-Pepper-1-III-20 | 501 | 590 | 39600 | 0.31 | 365 | 67.3 |

### Example 7. Labeling of Bacterial RNA Using Pepper-III-3 Complex

In order to test the effect of Pepper-III-3 in bacteria, a bacterial expression plasmid expressing F30-Pepper-1 was constructed. Primers were used to amplify F30-Pepper-1 in Example 2. Primers were used to amplify pET28a to remove the promoter and multiple cloning site regions. The obtained F30-Pepper-1 fragment and the linearized pET28a were ligated according to the commonly used experimental method (4). The obtained recombinant plasmid was named pET28a-T7-F30-Pepper-1.

The primers used to amplify the F30-Pepper-1 fragment are:

Forward primer (P1):
5′-GATCCCGCGAAATTAATACGACTCACTATAGGGTTGCCATGTGTA
TGTGGG-3′
Reverse primer (P2):
5′-CAAGGGGTTATGCTATTGCCATGAATGATCC-3′

The primers used to amplify and linearize the pET28a vector are:

Forward primer (P3)
5′-TAGCATAACCCCTTGGGGCCTCTAAACGGGTCTTGAG-3′
Reverse primer (P4):
5′-ATTTCGCGGGATCGAGATCTCGATCCTCTACGCCGGACG-3′

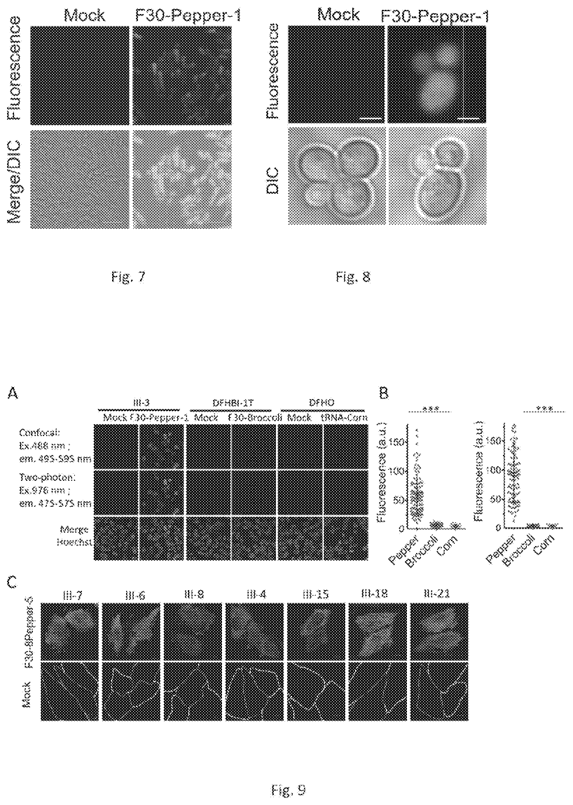

The recombinant plasmid pET28a-T7-F30-Pepper-1 was transformed into BL21(DE3, Star) E. coli strain. A single clone was picked and cultured at 37 C° to an OD₆₀₀ around 0.2 before addition of 1 mM IPTG to induce the expression of F30-Pepper-1. 4 hours after induction, the bacteria were harvested and resuspended in PBS solution containing 2 μM III-3. BL21 (DE3, Star) E. coli transformed with pET28a empty vector was used as the control. The results showed that bacteria exhibited bright yellow-green fluorescence only when F30-Pepper-1 was expressed and in the presence of III-3 (FIG. 7), indicating that Pepper-III-3 complex can be used for fluorescent labeling of RNA in bacteria.

### Example 8. Labeling of Yeast RNA Using Pepper-III-3 Complex

In order to test the effect of Pepper-III-3 in yeast, a yeast expression plasmid expressing F30-Pepper-1 was constructed. The F30-Pepper-1 DNA fragment in Example 2 was amplified using primers, and the amplified F30-Pepper-1 fragment was inserted into the pYES2.1TOPO TA vector according to the commonly used experimental method (4). The obtained recombinant plasmid was named pYES2.1-F30-Pepper-1.

The primers used to amplify the F30-Pepper-1 fragment are:

Forward primer (P5):
5’-GGAATATTAAGCTCGCCCTTTTGCCATGTGTATGTGGG-3’
Reverse primer (P6):
5’-TGACCTCGAAGCTCGCCCTTGTTGCCATGAATGATCC-3’

The recombinant plasmid pYES2.1-F30-Pepper-1 was transformed into BY4741 strain, and a single clone was picked and cultured at 30 °C to an OD600=0.1 before addition of 1 mM galactose to induce the expression of F30-Pepper-1. 10 hours after induction, the yeast cells were harvested and resuspended in PBS containing 2 μM III-3. The untreated BY4741 strain was used as the control. The results showed that yeast cells exhibited bright yellow-green fluorescence only when F30-Pepper-1 was expressed and in the presence of III-3 (FIG. 8), indicating that Pepper-III-3 complex can be used for RNA labeling in yeast cells.

### Example 9. RNA Labeling in Mammalian Cells Using Pepper and III-3 and its Analogs

In order to use Pepper and III-3 for RNA labeling in mammalian cells, the reported Broccoli and Corn aptamer (binding to DFHBI-1T and DFHO fluorophores, respectively) were used as the controls (Filonov et al. Journal of the American Chemical Society 2014. 136: 16299-16308; Song et al. Nature chemical biology 2017. 13: 1187-1194). The mammalian cell expression plasmids expressing the RNA aptamers were constructed. The primers P7 and P8 were used to amplify F30-Pepper-1 and F30-Broccoli in Example 2, and the primers P9 and P10 were used to amplify the synthesized tRNA-Corn cDNA fragment (the RNA sequence was SEQ ID No: 20). The obtained fragments were ligated into the pLKO.1 puro vector according to the commonly used experimental method (4). The obtained expression vectors were named pLKO.1-F30-Pepper-1, pLKO.1-F30-Broccoli and pLKO.1-tRNA-Corn, which express F30-Pepper-1, F30-Broccoli and tRNA-Corn RNA, respectively.

The primers used to amplify F30-Pepper-1 and F30-Broccoli are:

Forward primer (P7):
5′-GGAAAGGACGAAACTCTAGATTGCCATGTGTATGTGGG-3′
Reverse primer (P8):
5′-TGTCTCGAGGTCGAGAATTCAAAAAAAGTTGCCATGAAT
GATCC-3′

The primers used to amplify tRNA-Corn are:

Forward primer (P9):
5′-GGAAAGGACGAAACTCTAGAGCCCGGATAGCTCAGTCGG-3′
Reverse primer (P10):
5′-TGTCTCGAGGTCGAGAATTCAAAAAAATGGCGCCCGAA
CAGGGACTTGCGAGCTCAGGATCCTTCCGTTTCGCACTGG-3′

In order to use Pepper and III-3 analogues for RNA labeling in mammalian cells, a mammalian expression plasmid expressing F30-8Pepper-5 was constructed. The primers P7 and P8 in this example were used to amplify the F30-8Pepper-5 fragment in Example 5, and the fragments was inserted into the pLKO.1 puro vector using the commonly used experimental method (4). The obtained expression vector was named pLKO.1-F30-8Pepper-5.

The pLKO.1-F30-8Pepper-5 plasmid was transfected into 293T/17 cells. Different III-3 analogues were added into the culture for labeling 24 hours after transfection. Fluorescence imaging was performed according to the commonly used experimental method (3). The results showed that different III-3 analogs could be used to specifically label cells expressing F30-8Pepper-5, but not the control cells without expression of F30-8Pepper-5 (FIG. 9C), indicating that Pepper and III-3 and its analogs can be used to label RNA in mammalian cells.

### Example 10. Construction of Pepper-Based Sensors

In order to construct Pepper-based sensors for detecting analytes, the nucleotides in the stem-loop region in the Pepper-1 (SEQ ID No: 2) structure were replaced with the aptamers that can specifically recognize and bind adenosine and guanosine (GTP), respectively. The aptamers Pepper-1 were fused by linkage with different lengths and different nucleotides (FIG. 10A). The RNA sensors were prepared according to the commonly used experimental method (1). The RNA sensors were incubated with III-3 fluorophore, and the fluorescence was measured in the presence or absence of adenosine or GTP using a multifunctional microplate reader. The results showed that, for the adenosine sensor, the activation fold could reach 88 folds when the linkage between the adenosine aptamer and Pepper-1 was linkage 2 in FIG. 10B. The corresponding sequence of the RNA sensor was SEQ ID No:21; for the GTP sensor, the activation fold could reach 10 folds when the linkage between the adenosine aptamer and Pepper-1 was linkage 3 in FIG. 10B. The corresponding sequence of the RNA sensor was SEQ ID No:22.

### Example 11. Tracking of RNA Localization in Cells by Pepper

In order to use Pepper to track RNA localization in cells, chimeric RNA expression plasmids in which Pepper was fused to different RNAs were constructed. cDNA encoding 4Pepper-7 (the sequence of the RNA aptamer is SEQ ID No: 23) was synthesized and amplified using primers. The obtained fragment was inserted into the HindIII and XhoI sites of pCDNA3.1 hygro(+) vector to generate pCDNA3.1 hygro(+)-4Pepper-7 plasmid. The GAPDH and TMED2 gene fragments (the gene sequences of GAPDH and TMED2 are shown in Genebank: BC009081 and BC025957, respectively) were synthesized and amplified using primers. The obtained GAPDH and TMED2 gene fragments were inserted into the NheI and HindIII sites of pCDNA3.1 hygro(+)-4Pepper-7 vector to generate pCDNA3.1 hygro(+)-GAPDH-4Pepper-7 and pCDNA3.1 hygro(+)-TMED2-4Pepper-7 plasmids that express GAPDH-4Pepper-7 and TMED2-4Pepper-7 chimeric RNAs, respectively. The sequences of the chimeric RNAs are SEQ ID Nos: 24 and 25, respectively.

The primers used to amplify 4Pepper-7 are:

Forward primer (P11):
5′-TAGCGTTTAAACTTAAGCTTCCCACGGAGGATCCCCAATC-3′
Reverse primer (P12):
5′-ACGGGCCCTCTAGACTCGAGCCCACGGAGGATCCCGGCGCC-3′

The primers used to amplify GAPDH are:

Forward primer (P13):
5′-GGAGACCCAAGCTGGCTAGCATGGGGAAGGTGAAGGTCGG-3′
Reverse primer (P14):
5′-GGATCCTCCGTGGGAAGCTTAACCATGCTCTAGCGAGTGTTA
CTCCTTGGAGGCCATGT-3′

The primers used to amplify TMED2 are:

Forward primer (P15):
5′-GGAGACCCAAGCTGGCTAGCATGGTGACGCTTGCTGAACT-3′
Reverse primer (P16):
5′-GGATCCTCCGTGGGAAGCTTAACCATGCTCTAGCGAGTTAAA
CAACTCTCCGGACTTC-3′

After construction of above plasmids, the inserted sequences were validated by sequencing to ensure correct insertion. The plasmids were extracted using a transfection-grade plasmid extraction kit for subsequent transfection experiments.

The pCDNA3.1 hygro(+)-GAPDH-4Pepper-7 and pCDNA3.1 hygro(+)-TMED2-4Pepper-7 plasmids constructed in this example were co-transfected with pCDNA3.1 hygro(+)-BFP into COS-7 cells, respectively. 24 hours after transfection, the cells were imaged according to the fluorescence imaging method described in the commonly used experimental method (3). The imaging results showed that the fluorescence of GAPDH-4Pepper-7-III-3 was mainly localized in the cytoplasm, while the fluorescence of TMED2-4Pepper-7-III-3 exhibited endoplasmic reticulum enrichment, which was consistent with previous studies and the results obtained by fluorescent-labeled in situ hybridization (FISH) (FIG. 11). These results indicate that Pepper can be used to track RNA location.

### Example 12. Detection of the Correlation Between mRNA and Protein by Pepper

In order to use Pepper to detect mRNA translation in cells, it was necessary to construct plasmids expressing different Pepper-fused mRNA. Primers were used to amplify mCherry and TagBFP gene fragments using mCherry2-N1 (Addgene: 54517) and EasyFusion T2A-H2B-TagBFP (Addgene: 113086) as the templates. The obtained gene fragments were inserted into the NheI and HindIII sites of pCDNA3.1 hygro(+)-GAPDH-4Pepper-7 vector to generate pCDNA3.1 hygro(+)-mCherry-4Pepper-7 and pCDNA3.1 hygro(+)-TagBFP-4Pepper-7 plasmids that encode mCherry-4Pepper-7 and TagBFP-4Pepper-7, respectively. The sequences of the chimeric RNAs are SEQ ID Nos: 26 and 27, respectively.

The primers used to amplify mCherry are:

Forward primer (P17):
5′-GGAGACCCAAGCTGGCTAGCATGGTGAGCAAGGGCGAGGAGG-3′
Reverse primer (P18):
5′-GGATCCTCCGTGGGAAGCTTAACCATGCTCTAGCGAGTTACTTG
TACAGCTCGTCCATG-3′

The primers used to amplify TagBFP are:

Forward primer (P19):
5′-GGAGACCCAAGCTGGCTAGCATGAGCGAGCTGATTAAGGA-3′
Reverse primer (P20):
5′-GGATCCTCCGTGGGAAGCTTCTCCCAAACCATGCTCTA
GCGAGTGTTAATTGAGCTTGTGCCCCA-3′

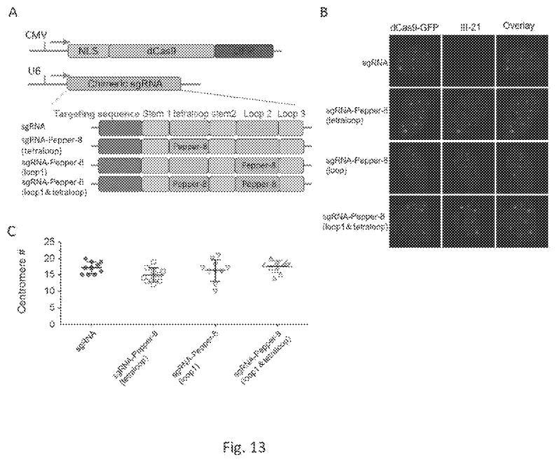

The recombinant plasmids pCDNA3.1 hygro(+)-BFP-4Pepper-7 and pCDNA3.1 hygro(+)-mCherry-4Pepper-7 were transfected into COS-7 cells, respectively. 24 hours after transfection, the transfected cells were labeled with 0.2 μM III-3 fluorophore. The fluorescence of mRNA (4Pepper-7-III-3) and fluorescence proteins (BFP and mCherry) was analyzed using flow cytometry. The fluorescence of mRNA and protein was fitted using the Michaelis equation to obtain R². The results showed that the translation efficiencies of different mRNAs varied significantly different (FIG. 12), indicating that Pepper can be used to detect the correlation between mRNA and protein.

### Example 13. Detection of Genomic DNA by Pepper

In order to use Pepper to detect genomic DNA, a recombinant plasmid expressing chimeric RNA of Pepper-8 and sgRNA was constructed. cDNAs encoding sgRNA-Pepper-8 (loop1), sgRNA-Pepper-8 (tetraloop) and sgRNA-Pepper-8 (loop1 and tetraloop) containing centromere targeting sequence were synthesized. The encoded RNA sequences are SEQ ID No: 28, 29 and 30, respectively. Primers P21 and P22 were used to amplify above cDNAs, primers P23 and P24 were used to amplify the psgRNA plasmid (Shao et al. Nucleic acids research 2016. 44: e86). The obtained cDNAs were inserted into the linearized psgRNA vector according to the commonly used experimental method (4) to generate psgRNA-Pepper-8 (loop 1), psgRNA-Pepper-8 (loop2) and psgRNA-Pepper-8 (loop1 and tetraloop), respectively (FIG. 13A). Primers P25 and P26 were used to amplify the dCas9-GFP gene fragment using pSLQ1645(dCas9-GFP) (Shao et al. Nucleic acids research 2016. 44: e86) as the template. The obtained gene fragment was inserted into the HindIII and XhoI sites of pCDNA3.1 hygro(+) vector to generate pCDNA3.1 hygro(+)-dCas9-GFP according to the commonly used experimental method (4).

The primers used to amplify the cDNA encoding Pepper and sgRNA chimeric RNA are:

Forward primer (P21):
5′-AAAGGACGAAACACCGAATCTGCAAGTGGATATTGTTTGAG-3′
Reverse primer (P22):
5′-TGATCTAGAAAAAAAGC ACCGACTCGGTGCCAC-3′

The primers used to amplify the psgRNA plasmid to linearize it are:

Forward primer (P23):
5′-TTTTTTTCTAGATCATAATCAGCCATACC-3′
Reverse primer (P24):
5′-GGTGTTTCGTCCTTTCCACAAG-3′

The primers used to amplify SpdCas9-GFP are:

Forward primer (P25):
5′-TAGCGTTTAAACTTAAGCTTGTGCAGGCTGGCGCCAC
CATGGCCCC-3′
Reverse primer (P26):
5′-ACGGGCCCTCTAGACTCGAGTTACTTGTACAGCTCGTC
CATGC-3′

pCDNA3.1 hygro(+)-dCas9-GFP and psgRNA-Pepper-6 (loop1), psgRNA-Pepper-6 (loop2) and psgRNA-Pepper-6 (loop1 and tetraloop) were co-transfected into COS-7 cells, respectively. 24 hours after transfection, the cells were labeled with 1 μM III-21 and Hoechst, and the fluorescence of Pepper-8-III-21, GFP and Hoechst were imaged using a fluorescence microscope. The imaging results showed that the fluorescence of Pepper-8-III-21 was mainly localized in the nucleus to exhibit aggregates in dots (centromeres), which was almost completely overlayed with the fluorescence of dCas9-GFP (FIG. 13B). Furthermore, the number of dots was consistent with the sgRNA alone (FIG. 13C), indicating that Pepper can be used to image genetic DNA.

### Example 14. Super-Resolution Imaging of RNA by Pepper

In order to use Pepper for super-resolution imaging of RNA, a plasmid that tethered RNA to the nucleus was constructed. The 4Pepper-9-MS2 DNA fragment (SEQ ID No: 31) was synthesized and amplified using primers P27 and P28. The obtained fragment was inserted into XbaI and EcoRI sites of pLKO.1 vector to generate pLKO.1-4Pepper-9-MS2 according to the commonly used experimental method (4). Primers were used to amplify H2B gene fragment using pCS-H2B-EGFP (Addgene: 53744) as the template, primers were used to amplify tdMCP gene fragments using pHAGE-Ubc-NLS-HA-tdMCP-GFP (Addgene: 40649) as the template, primers were used to amplify tagBFP gene fragment, the overlap PCR was used to fuse tdMCP, tagBFP and H2B gene fragments to obtain the tdMCP-tagBFP-H2B fusion fragment. The obtained tdMCP-tagBFP-H2B fragment was inserted into pmTurquoise2-Golgi (Addgene: 36205) to generate pH2B-tdMCP-tagBFP that encodes a nuclear-localized tdMCP-tagBFP according to the commonly used experimental method (4).

The primers used to amplify the 4Pepper-9-MS2 DNA fragment are:

Forward primer (P27):
5′-GGAAAGGACGAAACTCTAGAGGGGCCCCCCAATCGTGG-3′
Reverse primer (P28):
5′-TGTCTCGAGGTCGAGAATTCAAAAAAAGGGGCCCCCGGCG
CCAGTG-3′

The primers used to amplify the tdMCP gene fragment are:

Forward primer (P29):
5′-GAACCGTCAGATCCGCTAGCCACCATGGGCTACCCC
TACGACGTGCCCG-3′
Reverse primer (P30):
5′-TCCAGAATCCGCGTAGATGCCGG-3′

The primers used to amplify the tagBFP gene fragment are:

Forward primer (P31):
5′-CTACGCGGATTCTGGAGGCGGTGGATCCATGAGCGA
GCTGATTAAGGAG-3′
Reverse primer (P32):
5′-AGATCTATTGAGCTTGTGCCCCAGTTTG-3′

The primers used to amplify H2B gene fragment are:

Forward primer(P33):
5′-CAAGCTCAATAGATCTATGCCTGAACCGGCAAAATCC-3′
Reverse primer (P34):
5′-GACTGCAGAATTCGAAGCTTACTTGGAGCTGGTGT
ACTTG-3′

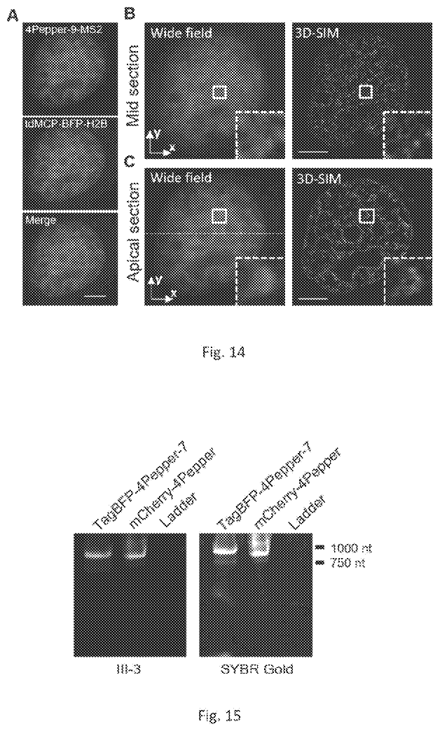

The pLKO.1-4Pepper-9-MS2 and pH2B-tdPP7-tagBFP plasmids were co-transfected into COS-7 cells. 24 hours after transfection, the transfected cells were labeled with III-21 fluorophore, and the fluorescence distribution of Pepper-III-21 complex was imaged using a Zeiss Elyra PS.1 super-resolution fluorescence microscope using an excitation of a 561 nm long-pass filter equipped with a Zeiss Plan-Apochromat 63× (NA, 1.4) Oil DIC M27 objective and a CMOS size of 1024×1024 pixels. The imaged were processed using a ZEN 2011 Black (Zeiss) software. The imaging results showed that the cells co-transfected with pLKO.1-4Pepper-9-MS2 and pH2B-tdMCP-tagBFP showed an obvious nucleopore structure (FIG. 14). The results indicate that Pepper can be used for super-resolution imaging of RNA.

### Example 15. RNA Extraction and Purification by Pepper

In order to use Pepper for RNA extraction and purification, the pCDNA3.1 hygro(+)-TagBFP-4Pepper-7 and pCDNA3.1 hygro(+)-mCherry-4Pepper-7 plasmids in Example 12 were transfected into COS-7 cells, respectively. 24 hours after transfection, the cells were collected and the total RNA of the cells was extracted using the Easyp Super Total RNA Extraction Kit (Promega). The extracted total RNA was dissolved in buffer containing 40 mM HEPES, pH 7.4, 125 mM KCl, 5 mM MgCl₂. The RNA was incubated at 70 °C for 10 min and placed at room temperature for more than 30 min.

500 uL activated Thiol Sepharose 4B (GE Healthcare) was washed twice with 500 μL PBS, and then was incubated with PBS containing 10 mM TCEP (Sigma) for 1 h at room temperature. After washing twice with 500 μL PBS, maleamide conjugated III-3 fluorophore (Mal-III-3) was added to react for 30 min at room temperature, and was washed three times with 500 μL PBS. The treated total RNA was incubated with the treated beads at room temperature. After 30 minutes, the mixture was centrifuged at 4000 rpm for 2 minutes, and the supernatant was discarded. The agarose beads were washed with buffer containing 40 mM HEPES, pH 7.4, 125 mM KCl, and 5 mM MgCl₂ for 6 times, and the supernatant was removed by centrifugation each time. The beads were resuspended with DEPC water, treated at 70 °C for 10 min, and centrifuged at 4000 rpm for 2 min. Then the supernatant was collected. 1/10 volume of NaAc, 2.5 times volume of absolute ethanol was added into the collected supernatant and placed in a refrigerator at −80 °C for 20 min. The mixture was centrifuged at 14000 rpm at 4 °C for 10 min. The precipitate was collected and the supernatant was discarded. The pre-cooled 70% ethanol solution was used to wash the precipitate. The mixture was then centrifuged at 14000 rpm for 10 min at 4 °C. The precipitate was collected and the supernatant was discarded. Such procedure was repeated once again. The precipitate was placed at room temperature for 5 minutes, and then a small volume of DEPC water was used to resuspend the precipitate after the alcohol was evaporated.

The recovered RNA was validated by electrophoresis. The gel was incubated with buffer containing 5 μM III-3 in 40 mM HEPES, pH 7.4, 125 mM KCl, and 5 mM MgCl₂ for 30 min, and the fluorescence of 4Pepper-III-3 in the gel was detected. The imaging results showed that two RNA bands in the gel exhibited bright Pepper-III-3 fluorescence signals, which were TagBFP-4Pepper and mCherry-4Pepper, respectively (FIG. 15), indicating that Pepper can be used as a tag for RNA extraction and purification.

### Example 16: The Synthesis of Compound III-1 and its Analogues Compound III-1

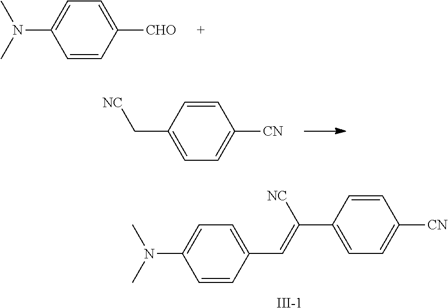

To a stirring solution of p-dimethylaminobenzaldehyde (0.35 g, 2.3 mmol) and 4-cyano-benzeneacetonitrile (0.4 g, 2.8 mmol) in 20 mL methanol, 2 drops of piperidine were added. After stirring at ambient temperature for 2 h, the mixture was cool to room temperature. A large amount of precipitate was appeared. Then the precipitate was obtained by filtration and washed with cold EtOH three times. The orange solid was obtained after dried under vacuum (0.60 g, yield 95%). ¹H NMR (400 MHz, DMSO-d₆): δ=3.05 (s, 6H), 6.83 (d, J=9.2 Hz, 2H), 7.84-7.94 (m, 6H), 8.02 ppm (s, 1H). HRMS (ESI-TOF) Calcd. For C₁₈H₁₆O₃ [M+H]⁺. 274.1344. Found: 274.1345.

### Compound III-2

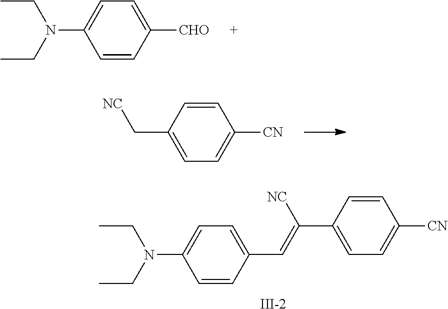

With reference to the synthetic method of compound III-1 (0.34, yield 89%). ¹H NMR (400 MHz, DMSO-d₆): δ=1.23 (t, J=7.60 Hz, 6H), 3.05 (t, J=7.60 Hz, 4H). 6.84 (d, J=9.2 Hz, 2H), 7.84-7.95 (m, 6H), 8.09 ppm (s, 1H). HRMS (ESI-TOF): Calcd. For C₂₀H₂₀O₃[M+H]⁺: 302.1657. Found: 302.1658.

### Compound III-3

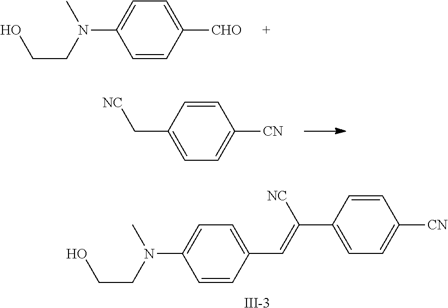

With reference to the synthetic method of compound III-1 (0.33 g, yield 95%). ¹H NMR (400 MHz, DMSO-d₆): δ=7.96 (s, 1H), 7.85 (d, J=16.0 Hz, 6H), 6.81 (d, J=8.0 Hz, 2H), 4.77 (s, 1H), 3.55 (d, J=28.0 Hz, 4H), 3.04 (s, 1H). HRMS (ESI-TOF) Calcd. For C₁₉H₁₈N₃O [M+H]⁺: 304.1450. Found 304.1451.

### Compound III-4

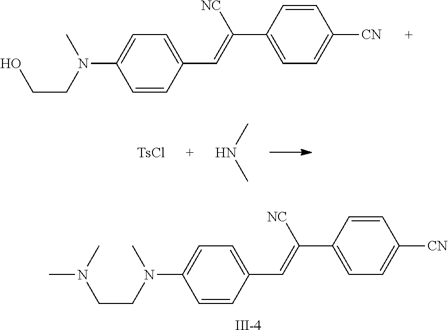

To stirring solution of compound III-3 (0.61 g, 2.0 mmol) and TEA (0.25 g, 2.2 mmol) in 40 mL dried DCM, 4-tosyl chloride (0.38 g, 2.0 mmol) in 10 mL DCM was added slowly under 0 °C. The resulting mixture was stirred under Ar atomo and was permitted to warm to room temperature. After complete the reaction, the mixture was quenched by 2 mL of water. The reaction mixture was extracted three times and the organic phase was dried with anhydrous Na₂SO₄ and evaporation under reduced pressure, the residue was used in the next step without purified.

To a stirring solution of the residue in 20 mL CH₃CN, 1 ml MeNH2 was added under Ar atmosphere. The mixture was heated to refluxed overnight. Upon completing the reaction, the reaction mixture was cooled to room temperature and the organic liquid was removed under reduce pressure. Then the residue was dissolved in 50 mL DCM and the organic phase was washed with water and brine (2×100 ml). Upon drying over anhydrous Na₂SO₄ and evaporation under reduced pressure, the residue was purified by column chromatography on silica gel to afford orangered solid. (0.54 g, 82%). ¹H NMR (400 MHz, CDCl₃): δ=7.88 (d, J=9.0 Hz, 2H), 7.74-7.65 (m, 4H), 7.48 (s, 1H), 6.73 (d, J=9.1 Hz, 2H), 3.60-3.55 (m, 2H), 3.08 (s, 3H), 2.57-2.52 (m, 2H), 2.34 (s, 6H). HRMS (ESI-TOF): Calcd. For C₂₁H₂₃N₄[M+H]⁺: 331.1923. Found: 331.1925.

### Compound III-5

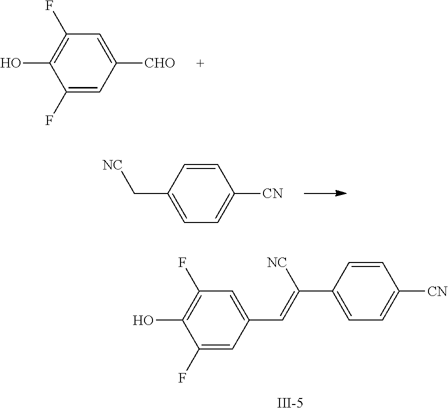

To a stirring solution of 3,5-difluoro-4-hydroxybenzaldehyde (0.32 g, 2.0 mmol) and 4-cyano-benzeneacetonitrile (0.35 g, 2.4 mmol) in 40 mL anhydrous EtOH, 2 drops of piperidine were added. After stirring at ambient temperature for 2 h, the mixture was cool to room temperature. A large amount of precipitate was appeared. Then the precipitate was obtained by filtration and washed with cold EtOH three times The orange solid was obtained after dried under vacuum. ¹H NMR (400 MHz, CDCl₃): δ=7.80 (d, J=9.0 Hz, 2H), 7.74-7.66 (m, 4H), 7.48 (s, 1H). HRMS (ESI-TOF): Calcd. For C₁₆H₉F₂N₂O [M+H]⁺: 283.0683. Found 283.0684.

### Compound Compound III-6

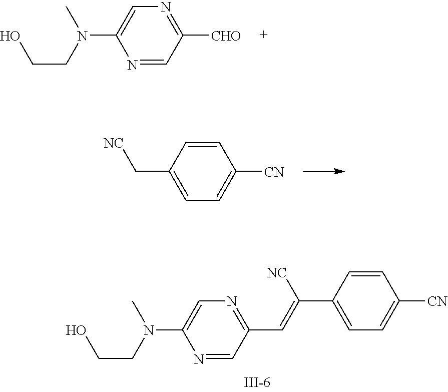

Wherein compound 5-(N-methyl-N-(2-hydroxyethyl)amino) pyrazine-2-carbaldehyde:

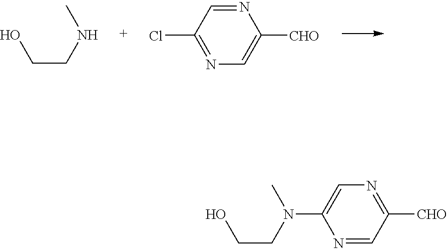

To a stirring solution of N-methyl-N-(2-hydroxyethyl)amino (2.6 g, 35 mmol) and 5-chloro-pyrazine-2-carbaldehyde (0.50 g, 3.5 mmol) in 20 m L dry CH₃CN, K₂CO₃ (0.71 g, 5.3 mmol) was added in one portion. The mixture was heated to reflux under Ar atmosphere. The mixture was heated to refluxed for 24 h. Upon completing the reaction, the reaction mixture was cooled to room temperature and the organic liquid was removed under reduce pressure. Then the residue was dissolved in 100 mL DCM and the organic phase was washed with water and brine (2×100 ml). Upon drying over anhydrous Na₂SO₄ and evaporation under reduced pressure, the residue was purified by column chromatography on silica gel to afford target compound. (0.48 g, 76%). ¹H NMR (400 MHz, CDCl₃): δ 9.88 (s, 1H), 8.62 (d, J=1.2 Hz, 1H), 8.14 (d, J=1.1 Hz, 1H), 3.92 (m, 2H), 3.88-3.83 (m, 2H), 3.28 (s, 3H). HRMS (ESI-TOF): Calcd. For C₈H₁₂N₃O₂ [M+H]⁺: 182.1. Found: 182.1.

Compound III-6 was synthesized with reference to the synthetic method of compound III-1 (0.36 g, 96%). ¹H NMR (400 MHz, CDCl₃): δ 8.39 (s, 1H), 8.30 (s, 1H), 7.80 (d, J=8.5 Hz, 2H), 7.72 (d, J=8.4 Hz, 2H), 7.51 (s, 1H), 3.93 (t, J=4.9 Hz, 2H), 3.88-3.83 (m, 2H), 3.29 (s, 31H1). HRMS (ESI-TOF). Calcd. For C₁₇H₁₆N₅O [M+H]⁺: 306.1355. Found: 306.1357.

### Compound III-7

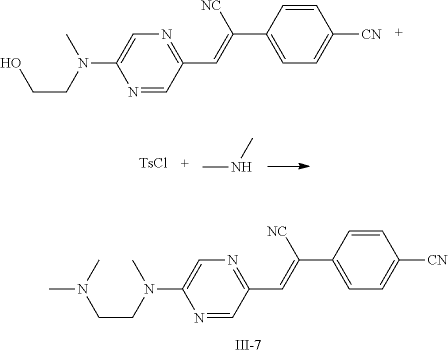

With reference to the synthetic method of compound III-4, (0.21 g, 67%), ¹H NMR (400 MHz, DMSO-d₆): δ 8.37 (d, J=5.2 Hz, 2H), 8.06 (s, 1H), 8.00-7.85 (m, 4H), 3.77 (t, J=6.5 Hz. 2H), 3.20 (s, 3H), 2.56 (m, 2H), 2.23 (s, 6H). HRMS (ESI-TOF): Calcd. For C1H₂₁N₆[M+H]⁺: 333.1828. Found: 333.1829.

### Compound III-8

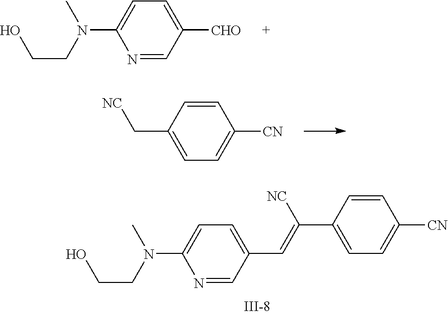

Wherein, compound 6-(N-methyl-N-(2-hydroxethyl)amino) pyridine-2-carbaldehyde:

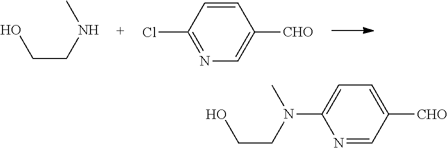

With reference to the synthetic method of Compound 5-(N-methyl-N-(2-hydroxyethyl)amino) pyrazine-2-carbaldehyde. (0.45 g. 68%). ¹H NMR (400 MHz, CDCl₃): δ=9.69 (s, 1H), 8.43 (d, J=2.1 Hz, 1H), 7.86 (dd, J=9.0, 2.3 Hz, 1H), 6.56 (d, J=9.1 Hz, 1H), 3.86-3.79 (m, 4H), 3.15 (s, 3H). HRMS (ESI-TOF): Calcd. For C₉₁H₁₃O₂N₂ [M+H]⁺. 181.1. Found 181.1.

Compound III-8 was synthesized with reference to the synthetic method of compound III-1. (0.39 g, 89%). ¹H NMR (400 MHz, DMSO-d₆): δ=8.54 (d, J=4.0 Hz, 1H), 8.30 (dd, J=9.3, 2.5 Hz, 1H), 8.03 (s, 1H), 7.92 (d, J=8.0 Hz, 2H), 7.85 (d, J=8.0 Hz, 2H), 6.84 (d, J=8.0 Hz, 1H), 4.77 (t, J=5.4 Hz, 1H), 3.67 (t, J=5.3 Hz, 2H), 3.60 (q, J=5.4 Hz, 2H), 3.15 (s, 3H). HRMS (ESI-TOF): Calcd. For C₁₈H₂₇N₄O [M+H]⁺: 305.1402. Found: 305.1401.

### Compound III-9

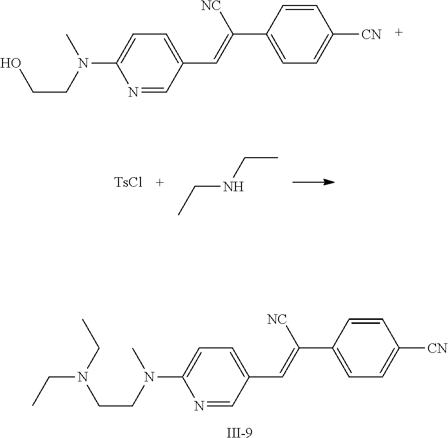

With reference to the synthetic method of compound III-4, (0.31 g, 92%). ¹H NMR (400 MHz, DMSO-d₆): δ=8.55 (d, J=4.0 Hz, 1H), 8.31 (dd, J=9.3, 2.5 Hz, 1H). 8.05 (s, 1H), 7.93 (d, J=8.0 Hz, 2H), 7.84 (d, J=8.0 Hz, 2H), 6.85 (d, J=8.0 Hz, 1H), 4.78 (t, J=5.4 Hz, 1H), 3.67 (t, J=5.3 Hz, 2H), 3.60 (q, J=5.4 Hz, 2H), 3.17 (t, J=8.0 Hz, 4H), 1.17 (t. J=8.0 Hz, 6H). HRMS (ESI-TOF): Calcd. For C₂₂H₂₆N₅ [M+H]⁺: 360.2188. Found 360.2187.

### Compound III-10

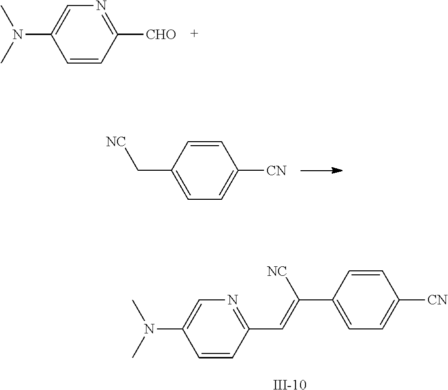

Wherein, Compound 4-(N,N-dimethylamino)-pyrazine-6-carbaldehyde

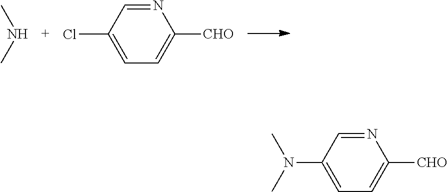

With reference to the synthetic method of compound III-4, (0.31 g, 49%). ¹H NMR (400 MHz, DMSO-d₆): δ=9.86 (d, J=0.6 Hz, 1H), 8.17 (d, J=2.9 Hz, 1H), 7.83 (d, J=8.9 Hz, 1H), 6.94 (dd, J=8.8, 2.9 Hz, 1H), 3.10 (s, 6H). HRMS (ESI-TOF): Calcd. For C₈H₁₁N₂O [M+H]⁺: 151.1 Found: 151.1.

Compound III-10 was synthesised with reference to the synthetic method of compound III-1, (0.36 g, 96%). ¹H NMR (400 MHz, DMSO-d₆): δ=9.86 (d, J=0.6 Hz, 1H), 8.26 (s, 11H), 8.17 (d, J=2.9 Hz, 11H1), 7.83 (d, J=89 Hz, 1H), 746 (m, 4H), 6.94 (dd, J=8.8, 2.9 Hz, 1H), 3.10 (s, 6H). HRMS (ESI-TOF): Calcd. For C₁₇H₁₅N₄ [M+H]⁺; 275.1297. Found: 275.1298.

### Compound 11

### Compound III-11

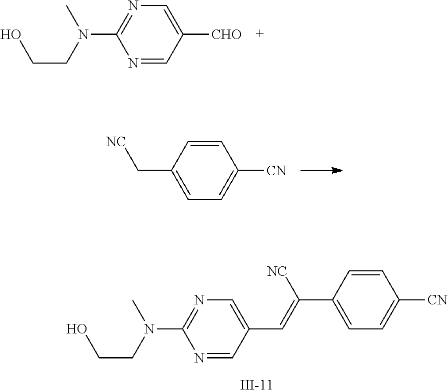

Wherein, compound 2-(N-methyl-N-(2-hydroxyethyl)amino) pyrimidine-5-carbaldehyde:

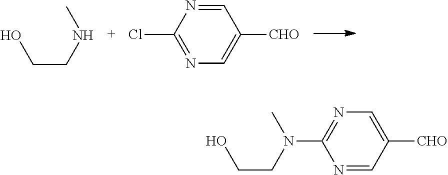

With reference to the synthetic method of compound III-4, (0.42 g, 72%). ¹H NMR (400 MHz, DMSO-d₆): δ=9.89 (s, 1H), 8.73 (s, 2H), 3.64 (t, J=8.9 Hz, 2H), 3.45 (t, J=8.8 Hz, 2H), 3.10 (s, 3H) HRMS (ESI-TOF): Calcd. For C₈H₁₂N₃O [M+H]⁺: 182.1. Found: 182.1.

Compound III-11 was synthesised with reference to the synthetic method of compound III-1, (0.36 g, 96%). ¹H NMR (400 MHz, DMSO-d₆) δ=8.26 (s, 1H), 8.73 (s, 2H), 7.64 (m, 4H), 3.64 (t, J=8.9 Hz, 2H), 3.44 (t, J=8.8 Hz, 2H), 3.11 (s, 3H). HRMS (ESI-TOF): Calcd. For C₁₇H₁₆N₅O [M+H]⁺: 306.1355. Found: 306.1356.

### Compound 12

### Compound III-12

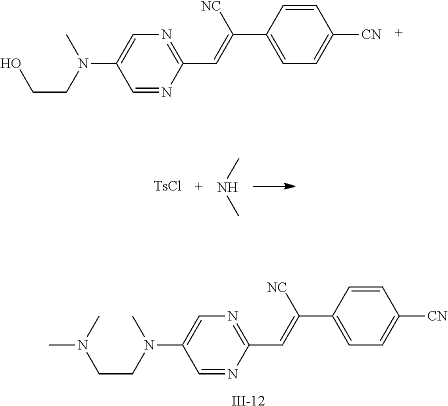

Wherein, compound 5-(N-methyl-N-(2-hydroxyethyl)amino) pyrimidine-2-carbaldehyde:

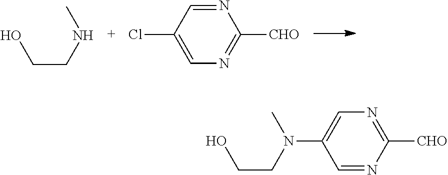

With reference to the synthetic method of compound III-4, (0.42 g, 72%). ¹H NMR (400 MHz, DMSO-d₆): δ=9.98 (s, 11H), 8.21 (s, 2H), 3.64 (t, J=8.9 Hz, 2H), 3.44 (t, J=8.8 Hz, 2H), 3.12 (s, 3H). HRMS (ESI-TOF): Calcd. For C₈H₁₂N₃O₂ [M+H]⁺: 182.1. Found: 182.1.

4-(1-cyano-2-(5-((2-hydroxyethyl)(methyl)amino)pyrimidin-2-yl)vinyl)benzonitrile1:

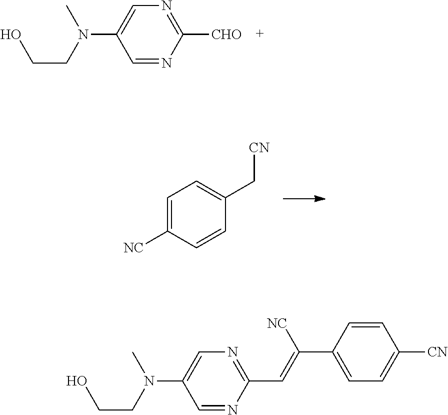 [SMILES: CN(CCO)c1cnc(C=O)nc1 ; N#CCc1ccc(C#N)cc1 ; CN(CCO)c1cnc(/C=C(\C#N)c2ccc(C#N)cc2)nc1]

With reference to the synthetic method of compound III-1, (0.56 g, 89%). ¹H NMR (400 MHz, DMSO-d₆): δ=8.21 (s, 2H), 7.99 (s, H), 7.64 (s, 4H), 3.64 (t, J=8.9 Hz, 2H), 3.44 (t, J=8.8 Hz, 2H), 3.12 (s, 3H). HRMS (ESI-TOF): Calcd. For C₁₇H₁₆N₅O [M+H]⁺: 306.1. Found: 306.1.

Compound III-12 was synthesised with reference to the synthetic method of compound III-4, (0.36 g, 96%). ¹H NMR (400 MHz, DMSO-d₆): δ=8.21 (s, 2H), 7.99 (s, 1H), 7.64 (s, 4H), 3.77 (t, J=6.5 Hz, 2H), 3.20 (s, 31H1), 2.56 (m, 2H), 2.23 (s, 6H). HRMS (ESI-TOF): Calcd. For C₁₉H₂₁N₆ [M+H]⁺: 333.1828. Found: 333.1829.

### Compound 13

### Compound III-13

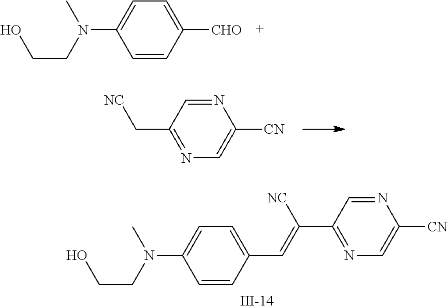

Wherein, 5-cyano-2-acetonitrile-pyridine:

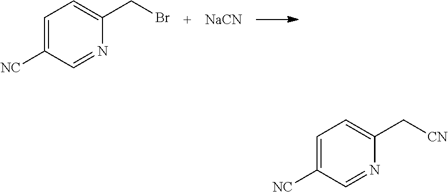

To a stirring solution of 2-(bromomethyl)-benzonitrile (0.50 g, 2.5 mmol) in 50 mL THF, 10 ml NaCN aqueous solution (2 M) was added. The mixture was reflexed for 12 h under Ar atmosphere. Upon cooling to room temperature, the reaction mixture was extracted with DCM (3×100 ml). The organic phase was washed with water and brine (2×100 ml). Upon drying over anhydrous Na₂SO₄ and evaporation under reduced pressure, the residue was purified by column chromatography on silica gel to afford target compound. (0.19 g, 56%. ¹H NMR (400 MHz, DMSO-d₆): δ=8.78 (s, 1H), 7.95 (m, 1H), 7.56 (m, 1H), 4.01 (s, 2H). HRMS (ESI-TOF): Calcd. For C₈H₆N₃ [M+H]⁺. 144.1. Found: 144.1.

Compound III-13 was synthesised with reference to the synthetic method of compound III-1, (0.45 g, 95%). ¹H NMR (400 MHz, DMSO-d₆): δ=8.78 (s, 1H), 8.21 (s, 1H), 7.94 (m, 1H), 7.86 (d, J=8.0 Hz, 2H), 7.57 (m, 1H), 6.80 (d, J=8.0 Hz, 2H), 3.64 (t, J=8.9 Hz, 2H), 3.44 (t, J=8.8 Hz, 2H), 3.12 (s, 3H). HRMS (ESI-TOF): Calcd. For C₁₈H₁₇N₄O [M+H]⁺: 305.1402 Found 305.1403.

### Compound 14

### Compound III-14

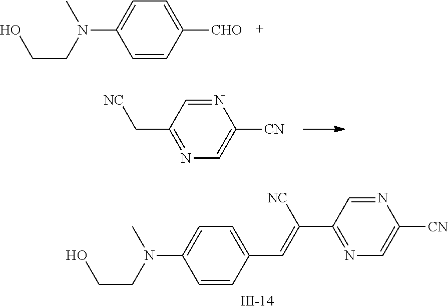

Wherein, 5-cyano-2-acetonitrile-pyrazine:

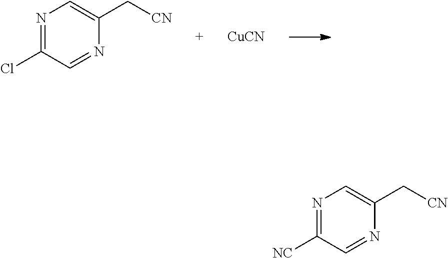

To a stirring solution of 2-(5-chloropyrazin-2-yl)acetonitrile (0.32 g, 2.0 mmol) in dry 30 mL DMSO, CuCN (0.93 g, 10.0 mmol) was added in one portation. The mixture was heated for 12 h under Ar atmosphere. Upon cooling to room temperature, the reaction mixture was poured into 100 mL water, then extracted with DCM (4×50 ml). The organic phase was washed with water and brine (2×100 ml). Upon drying over anhydrous Na₂SO₄ and evaporation under reduced pressure, the residue was purified by column chromatography on silica gel to afford target compound (0.20 g, 69%) ¹H NMR (400 MHz, DMSO-d₆) δ=8.60 (s, 1H), 8.48 (s, 1H), 3.92 (s. 2H). HRMS (ESI-TOF): Calcd. For C₇H₅N₄ [M+H]⁺: 145.1. Found: 145.1.

Compound III-14 was synthesised with reference to the synthetic method of compound III-1, (0.25 g, 91%). ¹H NMR (400 MHz, DMSO-d₆): δ=8.60 (s, 1H), 8.48 (s, 1H), 8.11 (s, 1H), 7.81 (d, J=8.2 Hz, 2H), 6.84 (d, J=8.2 Hz, 2H), 3.60 (t, J=9.2 Hz, 2H), 3.46 (t, J=9.2 Hz, 2H), 3.12 (s, 3H). HRMS (ESI-TOF): Calcd. For C₁₇H₁₆N₅O [M+H]⁺: 306.1355. Found: 306.1354.

### Compound 15

### Compound III-15

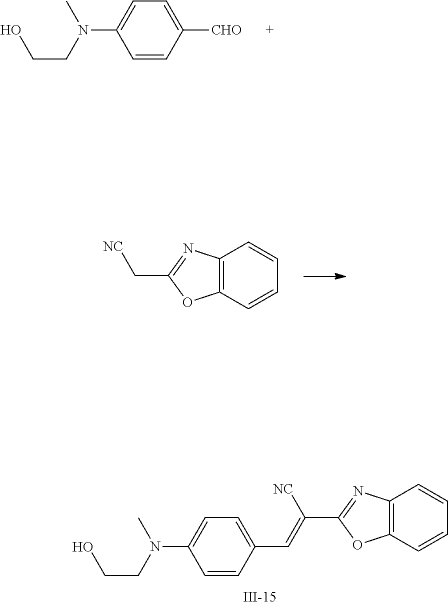

With reference to the synthetic method of compound III-1, (0.25 g, 91%). ¹H NMR (400 MHz, DMSO-d₆): δ=8.22 (s, 1H), 8.00 (d, J=9.1 Hz, 1H), 7.77-7.69 (m, 1H), 7.43-7.34 (m, 1H), 6.88 (d, J=9.1 Hz, 1H), 4.81 (t, J=5.2 Hz, 1H), 3.64-3.52 (m, 3H), 3.09 (s, 1H). LR-HRMS (ESI-TOF): Calcd. For C₁₉H₁₈N₃O₂ [M+H]⁺: 320.1399 Found: 320.1397.

### Compound III-16

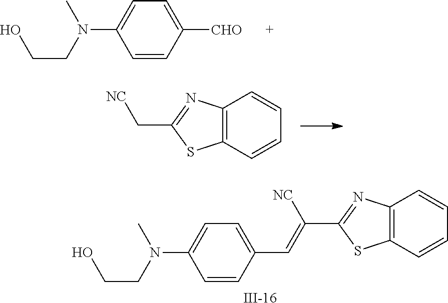

With reference to the synthetic method of compound III-1, (0.29 g, 94%). ¹H NMR (400 MHz, DMSO-d₆). δ=8.11 (2H, d, J=10.4 Hz), 7.99 (3H, dd, J=8.6, 3.0 Hz), 7.54 (1H, dd, J=8.0, 8.0 Hz), 7.44 (1H, dd, J=8.0, 8.0 Hz), 6.88 (2H, d, J=9.2 Hz), 4.82 (1H, bt, t, J=5.2 Hz), 3.60 (2H, t, J=5.2 Hz), 3.56 (2H, t, J=5.2 Hz), 3.09 (3H, s). LR-HRMS (ESI-TOF). Calcd. For C₁₉H₁₈N₃OS [M+H]⁺: 336.1171. Found: 336.1170.

### Compound III-17

### Compound III-17

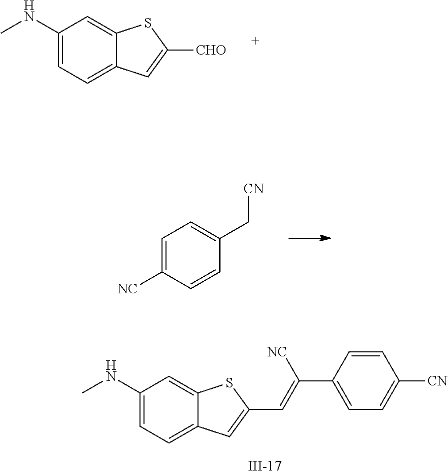

Wherein, 6-(methylamino)benzo[b]thiophene-2-carbaldehyde:

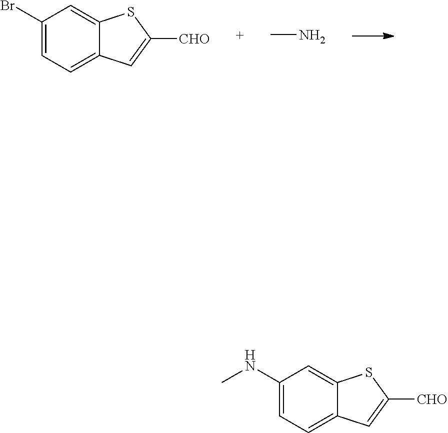

6-(methylamino)benzo[b]thiophene-2-carbaldehyde (0.42 g, 1.7 mmol), 40% aqueous N,N-Dimethylethylamine solution (1 g, 8.9 mmol), CuI (13.9 mg, 0.073 mmol), K₃PO₄·H₂O (155.4 mg, 0.73 mmol)), 1 mL 33% aqueous methylamine solution and stirring bar was sealed in a screwed tube and stirred at 60 °C for 12 h. upon cooling to room temperature, the mixture was poured into 50 mL water. The organic layer was separated and the aqueous layer was extracted with DCM (3×100 ml). Combined the organic phase and dried over anhydrous Na₂SO₄ and evaporation under reduced pressure, the residue was purified by column chromatography on silica gel to afford target compound (0.23 g, 68%) ¹H NMR (400 MHz, DMSO-d₆): δ=9.92 (1H, s), 8.14 (1H, s), 7.82 (1H, d, J=9.1 Hz), 7.18 (1H, d, J=2.1 Hz), 7.01 (1H, dd, J=9.1, 2.3 Hz), 3.05 (3H, s). HRMS (ESI-TOF): Calcd. For C₁₀H₁₀NOS [M+H]⁺: 192.0. Found. 192.0.

Compound III-17 was synthesised with reference to the synthetic method of compound III-1, (0.29 g, 94%). ¹H NMR (400 MHz, DMSO-d₆): δ=8.45 (s, 1H), 7.92 (d, J=8.6 Hz, 2H), 7.85 (d, J=8.3 Hz, 3H), 7.73 (dd, J=8.6, 3.9 Hz, 11H1), 7.21 (d, J=1.9 Hz, 1H), 7.21 (d, J=1.9 Hz, 1H), 6.96 (dd, J=9.1, 2.3 Hz, 1H), 3.05 (s, 3H). HRMS (ESI-TOF): Calcd. For C₁₉H₁₄N₃S [M+H]⁺: 360.1171. Found: 360.1173.

### Compound III-18

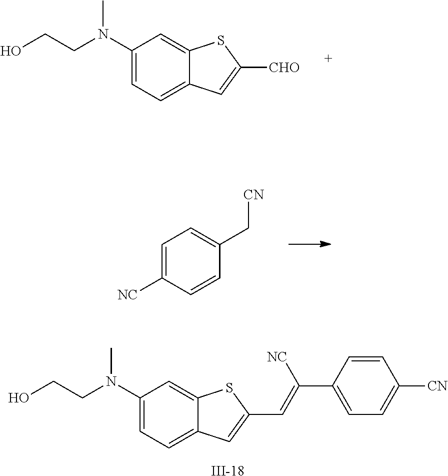

Wherein, 6-((2-hydroxyethyl)(methyl)amino)benzo[b]thiophene-2-carbaldehyde:

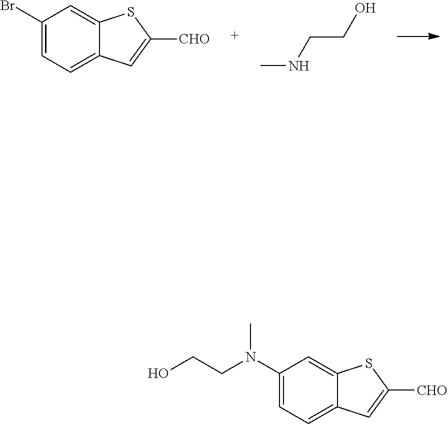

With reference to the synthetic method of compound 6-(methylamino)benzo[b]thiophene-2-carbaldehyde, (0.54 g, 79%). ¹H NMR (400 MHz, DMSO-d₆): δ=9.91 (s, 1H), 8.14 (s, 1H), 7.81 (d, J=5.2 Hz, 1H), 7.17 (d, J=2.0 Hz, 1H), 7.01 (dd, J=2.0, 8.8 Hz, 1H), 4.76 (t, J=5.6 Hz, 1H), 3.58 (t, J=4.2 Hz, 2H), 3.52 (t, J=4.2 Hz, 2H), 3.04 (s, 3H). HRMS (ESI-TOF): m/z Calcd. For C₁₂H₁₄NO₂S, [M+H]⁺: 235.1 Found 236.1.

Compound III-18 was synthesised with reference to the synthetic method of compound III-1, (0.21 g, 95%). ¹H NMR (400 MHz, DMSO-d₆): δ=8.45 (s, 1H), 7.92 (d, J=8.6 Hz, 2H), 7.85 (d, J=8.3 Hz, 3H), 7.73 (dd, J=8.6, 3.9 Hz, 1H), 7.21 (d, J=1.9 Hz, 1H), 7.21 (d, J=1.9 Hz, 1H), 6.96 (dd, J=9.1, 2.3 Hz, 1H), 3.63-3.57 (m, 2H), 3.52 (t. J=5.7 Hz, 2H), 3.05 (s, 3H). HRMS (ESI-TOF): Calcd. For C₂₁H₁₉N₃OS [M+H]⁺: 360.1171. Found: 360.1173.

### Compound III-19

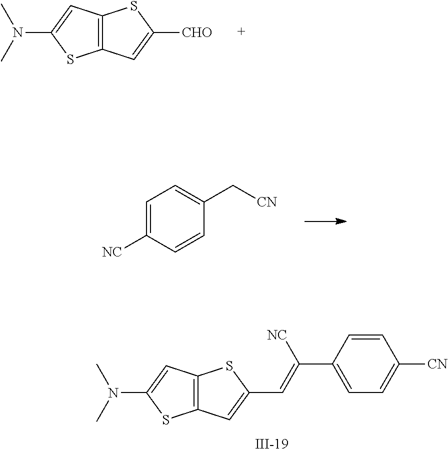

Wherein, 5-(N,N-dimethylamino)-thieno[3,2-b]thiophene-2-carbaldehyde:

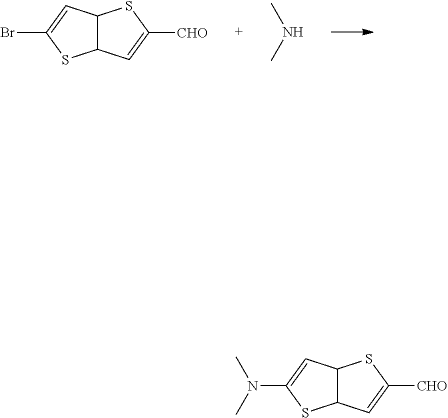

With reference to the synthetic method of compound 6-((2-hydroxyethyl)(methyl)amino)benzo[b]thiophene-2-carbaldehyde, (0.54 g, 79%). ¹H NMR (400 MHz, DMSO-d₆): δ=9.66 (s, 1H), 8.05 (s, 1H), 6.30 (s, 1H), 4.88 (bt, 1H), 3.07 (s, 6H) HRMS (ESI-TOF): m/z Calcd. For C₉H₁₂ NOS₂ [M+H]⁺: 214.0; found 214.0.

Compound III-19 was synthesised with reference to the synthetic method of compound III-1, (0.31 g, 90%). ¹H NMR (400 MHz, DMSO-d₆): δ=8.34 (s, 1H), 7.86 (d, J=8.0 Hz, 2H), 7.81 (s, 1H), 7.77 (d, J=8.0 Hz, 2H), 6.32 (s, 1H), 4.88 (t, J=4.0 Hz, 1H), 3.08 (s, 6H). HRMS (ESI-TOF): Calcd. For C₁₈H₁₄N₃S₂ [M+H]⁺: 336.0629. Found: 336.0630.

### Compound III-20

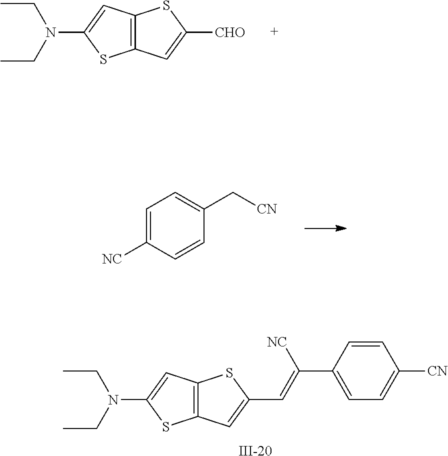

Wherein, 5-(N,N-diethylamino)-thieno[3,2-b]thiophene-2-carbaldehyde:

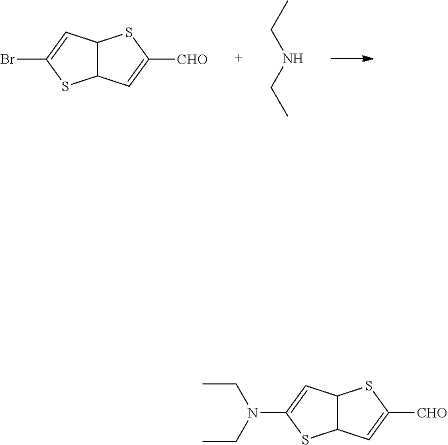

With reference to the synthetic method of compound 5-(N,N-dimethylamino)-thieno[3,2-b]thiophene-2-carbaldehyde, (0.44 g, 75%). ¹H NMR (400 MHz, DMSO-d₆): δ=9.78 (s, 1H), 8.09 (s, 1H), 6.30 (s, 1H), 4.87 (bt, 1H), 3.27 (t, J=8.4 Hz, 4H), 1.26 (t, J=8.4 Hz, 4H). HRMS (ESI-TOF): m/z Calcd. For C₉H₁₂ NOS₂ [M+H]⁺: 214.0; found 214.0.

Compound III-20 was synthesised with reference to the synthetic method of compound III-1, (0.31 g, 90%). ¹H NMR (400 MHz, DMSO-d₆): δ=8.34 (s, 1H), 7.86 (d, J=8.0 Hz, 2H), 7.81 (s, 1H), 7.77 (d, J=8.0 Hz, 2H), 6.32 (s, 1H), 4.88 (t, J=4.0 Hz, 1H), 3.27 (t, J=8.4 Hz, 4H), 1.26 (t, J=8.4 Hz, 4H). HRMS (ESI-TOF): Calcd. For C₂₀H₁₈N₃S₂[M+H]⁺: 364.0942. Found: 364.0943.

### Compound III-21

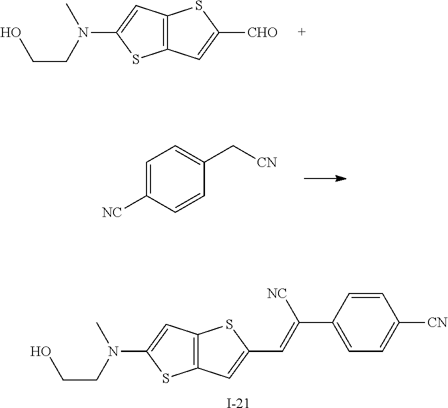

Wherein,

5-((2-hydroxethyl)(methyl)amino)-thieno[3,2-b]thiophene-2-carbaldehyde:

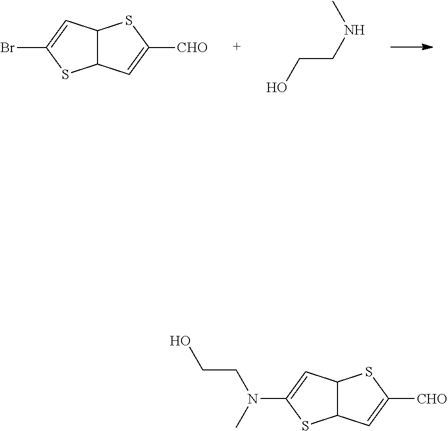

With reference to the synthetic method of compound 6-((2-hydroxyethyl)(methyl)amino)benzo[b]thiophene-2-carbaldehyde, (0.44 g, 75%). ¹H NMR (400 MHz, DMSO-d₆): δ=9.66 (s, 1H), 8.05 (s, 1H), 6.30 (s, 1H), 4.88 (bt, 1H), 3.64 (t, J=5.6 Hz, 2H), 3.44 (t, J=5.6 Hz, 2H), 3.07 (s, 3H). HRMS (ESI-TOF): m/z Calcd. For C₁₀H₁₂NO₂S₂[M+H]⁺: 241.0; found 242.0.

Compound III-21 was synthesised with reference to the synthetic method of compound III-1, (0.31 g, 90%). ¹H NMR (400 MHz, DMSO-d₆): δ8.34 (s, 1H), 7.86 (d, J=8.0 Hz, 2H), 7.81 (s, 1H), 7.77 (d, J=8.0 Hz, 2H), 6.32 (s, 1H), 4.88 (t, J=4.0 Hz, 1H), 3.65 (q, J=5.5 Hz, 2H), 3.44 (t, J=5.5 Hz, 2H), 3.34 (s, 1H), 3.08 (s, 3H). HRMS (ESI-TOF): Calcd. For C₁₉H₁₆N₃OS₂ [M+H]⁺: 366.0735. Found: 366.0736.

It will be understood that the dosages, reaction conditions, etc., in the examples are approximate values unless noted otherwise, and they can be exactly changed base on the situations to obtain similar results. All of the professional terms used in the Description, except those specially defined, have identical meanings to those known by persons skilled in the art. All the references referred to are incorporated into the application as a whole. The preferable embodiments are only exemplified for the illustration of the invention. Those skilled in the art can adopt similar methods or materials to obtain similar results. All the changes and modifications are within the scope of the attached claims.

What is claimed is:

1. A nucleic acid aptamer molecule containing following nucleotide sequences (a), (b) or (c), wherein
(a): a nucleotide sequence NiCCAAUCGUGGCGUGUCGN₁₉-N₂₀-N₂₁ACUGGCGCCGN₃₂, wherein N₁, N₁₉, N₂₀, N₂₁ and N₃₂ represent nucleotide fragments greater than or equal to 1 in length, and at least one base pair in N₁ and N₃₂ nucleotide sequences forms a complementary pair, and at least one base pair in N₁₉ and N₂₁ nucleotide sequences forms a complementary pair;
(b): a nucleotide sequence with an identity of at least 70% to the nucleotide sequence defined by (a); and
(c): a nucleic acid aptamer molecule derived from (a) at a position not including N₁, N₁₉, N₂₀, N₂₁ and N₃₂ in the nucleotide sequence defined by (a), with substitution, missing and/or addition of one or several nucleotides, and having an aptamer function.

2. The nucleic acid aptamer molecule according to claim 1, wherein, when N₁ and N₃₂ in the nucleotide sequence (a) form a complementary pair, a direction of N₁ nucleotide sequence is 5′-3′, and a direction of N₃₂ nucleotide sequence is 3′-5′; and when N₁₉ and N₂₁ form a complementary pair, a direction of N₁₉ nucleotide sequence is 5′-3′, and a direction of N₂₁ nucleotide sequence is 3′-5′.

3. The nucleic acid aptamer molecule according to claim 2, wherein, when at least one fragment of N₁ and N₃₂ is greater than or equal to 5 nucleotide bases in length, at least two base pairs in N₁ and N₃₂ nucleotide sequences form complementary pairs; when at least one fragment of N₁₉ and N₂₁ is greater than or equal to 5 nucleotide bases in length, at least two base pairs in N₁₉ and N₂₁ nucleotide sequences form complementary pairs.

4. The nucleic acid aptamer molecule according to claim 1, wherein the nucleotide substitution in the nucleotide sequence (a) is selected from one of the following groups: C3A, C3U, A4U, A4G, A4C, A5G, A5C, U6A, U6G, U6C, C7A, C7U, G8C, U9A, G11A, G11U, C12G, C12A, C12U, G13C, U14A, U14G, C17U, G18U, G18C, C27G, C27U, G28U, C29G, C29U, C30A, C30U, C2G/G31C, C2U/G31A, C2A/G31U, G10A/C30U, G10C/C30G, G10U/C30A, C2G/G31C/C3A, C2G/G31C/A4C, C2G/G31C/A5C, C2G/G31C/G8C, C2G/G31C/C12U, C2G/G31C/U14G, C2G/G31C/C27U, C2G/G31C/C29G, C2G/G31C/C30U, C2G/G31C/G10A/C30U, C2G/G31C/G10C/C30G, C2G/G31C/G10U/C30A, C2U/G31A/G10A/C30U, C2U/G31A/G10C/C30G, C2U/G31A/G10U/C30A, C2A/G31U/G10A/C30U, C2A/G31U/G10C/C30G, C2A/G31U/G10U/C30A, C2G/G31C/G10C/C30G/C3A, C2G/G31C/G10C/C30G/A4C, C2G/G31C/G10C/C30G/A5C, C2G/G31C/G10C/C30G/G8C, C2G/G31C/G10C/C30G/C12U, C2G/G31C/G10C/C30G/U14G, C2G/G31C/G10C/C30G/C27U, C2G/G31C/G10C/C30G/C29G, C2G/G31C/G10A/C30U/U6G/C27U, C2G/G31C/G10C/C30G/U6G/C27U, C2G/G31C/G10U/C30A/U9A/U14G/C27U and C2A/G31U/G10U/C30A/U9A/U14G/C27U.

5. The nucleic acid aptamer molecule according to claim 1, wherein nucleotide sequences at N₁ and N₃₂ in the nucleotide sequence (a) are F30 or tRNA scaffold RNA sequences.

6. The nucleic acid aptamer molecule according to claim 1, wherein the nucleic acid aptamer molecules are RNA molecules or RNA base-modified molecules.

7. The nucleic acid aptamer molecule according to claim 1, wherein the nucleic acid aptamer molecules are DNA-RNA hybrid molecules or DNA-RNA base-modified molecules.

8. The nucleic acid aptamer molecule according to claim 1, wherein N₁₉-N₂₀-N₂₁ in the nucleotide sequence (a) contains a nucleotide sequence capable of identifying target molecules.

9. The nucleic acid aptamer molecule according to claim 8, wherein the target molecules is at least one of proteins, nucleic acid, lipid molecules, carbohydrates, hormones, cytokines, chemokines, and metabolite metal ions.

10. The nucleic acid aptamer molecule according to claim 8, wherein N₁₉-N₂₀-N₂₁ in the nucleotide sequence (a) is a nucleotide sequence capable of identifying GTP and adenosine molecules.

11. The nucleic acid aptamer molecule according to claim 1, wherein the aptamer function refers to that the nucleic acid aptamer can enhance fluorescence intensity of fluorophore molecules under excitation light of appropriate wavelength by at least two times, at least 5 to 10 times, at least 20 to 50 times, at least 100 to 200 times or at least 500 to 1,000 times.

12. The nucleic acid aptamer molecule according to claim 1, further containing: concatemers that can bind multiple fluorophore molecules, and the concatemers are connected by spacer sequences of appropriate length having 2, 3, 4, 5, 6, 7, 8 or more nucleotide fragments. Nucleotides of the concatemers can be selected from but are not limited to a sequence SEQ ID No: 8, 9, 10, 11, 12, 13, 14, 15, 16, 17, 18 and 19.

13. A complex of nucleic acid aptamer molecules and fluorophore molecules, wherein the nucleic acid aptamer molecules are the nucleic acid aptamer molecules according to claim 1, the fluorophore molecules having a structure shown in Formula (I) below: 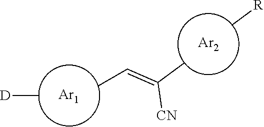
wherein: D- is X₁O— or N(X₂)(X₃)—; X₁, X₂, X₃ are respectively and independently selected from hydrogen, straight or branched alkyl and modified alkyl with 1 to 10 carbons, and X₂ and X₃ are optionally interconnected to be a satured or unsatured ring; R— is selected from hydrogen, cyano group, carboxyl, amide group, ester group, hydrixy, and straight or branched alkyl or modified alkyl with 1 to 10 carbons; Ar₁ and Ar₂ are respectively and independently selected from monocyclic aryl subunits, monocyclic heteraryl subunits, or aromatic subunits with 2 to 3 ring structures condensed from one or both of monocyclic aryl group and monocyclic heteroaryl group;
wherein: hydrogen atoms in Ar₁, and Ar₂ can be independently substituted by F, Cl, Br, I, hydroxyl group, nitro group, aldehyde group, carboxyl group, cyano group, sulfonic acid group, sulfuric acid group, phosphoric acid group, amino group, primary amino group, secondary amino group, straight or branched alkyl and modified alkyl with 1 to 10 carbons;
wherein: the modified alkyl is a group obtained by replacing any carbon atom of the alkyl with at least one group selected from F, Cl, Br, I, —O—, —OH, —CO—, —NO₂, —CN, —S—, —SO₂—, —(S═O)—, azide group, phenylene, primary amino group, secondary amino group, tertiary amino group, quaternary ammonium group, ethylene oxide, succinate, isocyanate, isothiocyanate, acyl chloride, sulfonyl chloride, saturated or unsaturated monocyclic or dicyclic subcyclic hydroxyl, bridged ester heterocycle, and the modified alkyl has 1 to 10 carbon atoms, wherein the carbon-carbon single bond is optionally and independently replaced by a carbon-carbon double bond or a carbon-carbon triple bond; and
wherein the nucleic acid aptamer molecules and the fluorophore molecules in the complex respectively exist in an individual solution, or the nucleic acid aptamer molecules and the fluorophore molecules are in the same solution.

14. The complex according to claim 13, wherein the modified alkyl contains at least one group selected from —OH, —O—, glycol unit, monosaccharide unit, disaccharide unit, —O—CO—, —NH—CO—, —SO₂—O—, —SO—, Me₂N—, Et₂N—, —S—S—, —CH═CH—, F, Cl, Br, I, —NO₂ and cyano group;
the aromatic rings contained in the fluorophore molecules being selected from structures represented by the following formulae (II-1) to (II-15):

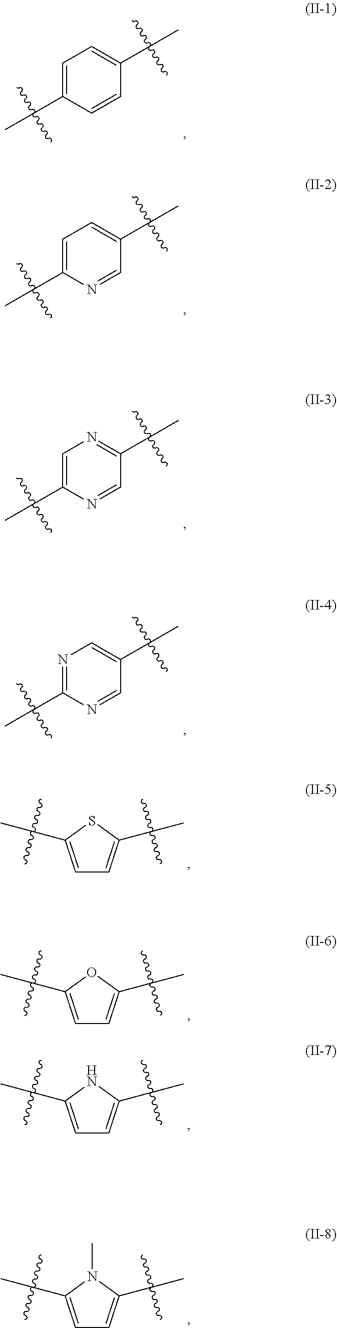

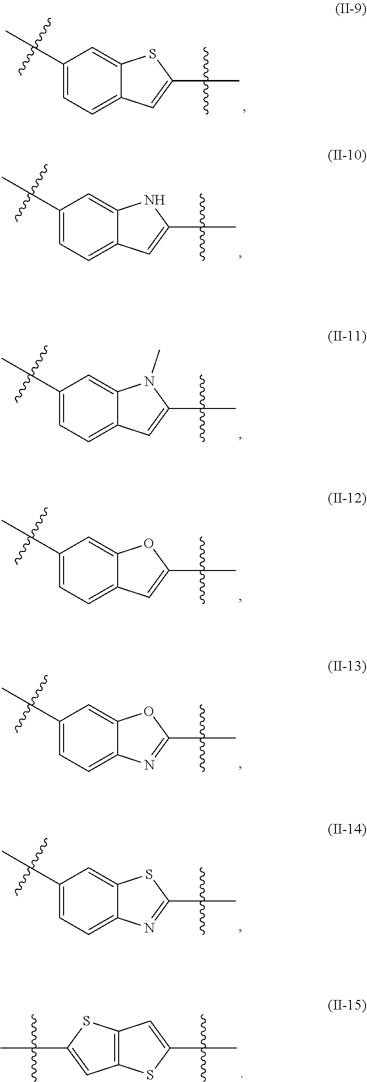

15. The complex according to claim 13, wherein fluorophore molecules are selected from the following compounds represented by the following formulae:

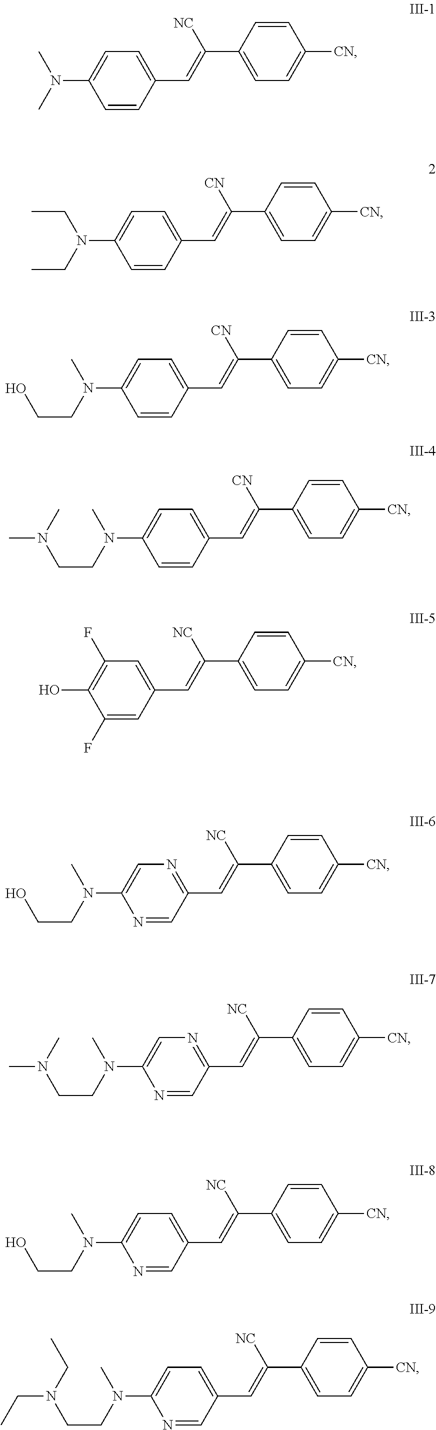

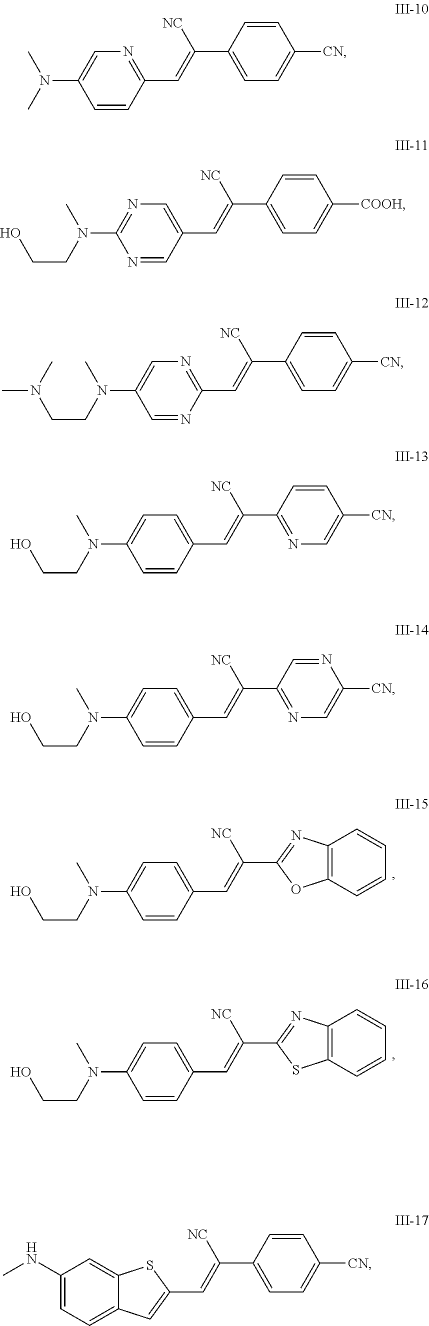

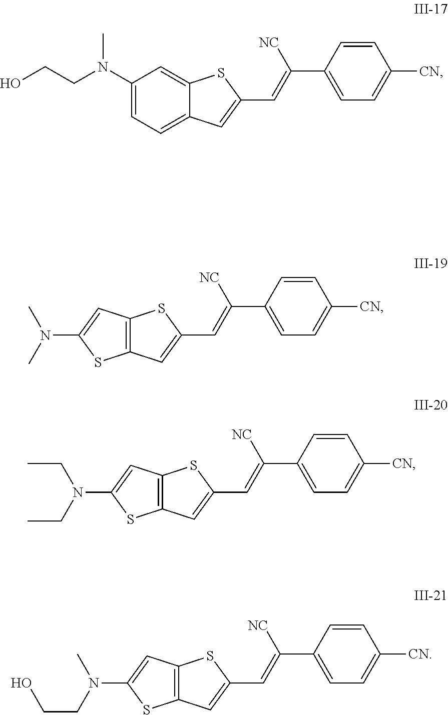 [SMILES: CN(CCO)c1ccc2cc(/C=C(\C#N)c3ccc(C#N)cc3)sc2c1 ; CN(C)c1cc2sc(/C=C(\C#N)c3ccc(C#N)cc3)cc2s1 ; CCN(CC)c1cc2sc(/C=C(\C#N)c3ccc(C#N)cc3)cc2s1 ; CN(CCO)c1cc2sc(/C=C(\C#N)c3ccc(C#N)cc3)cc2s1]

16. The complex according to claim 13, wherein the aptamer molecules in the complex contain nucleotide sequence SEQ ID No: 1, 2, 3, 6, 7, 8, 9, 10, 11, 12, 13, 14, 15, 16, 17, 18, 19, 21, 22, 23, 24, 25, 26, 27, 28, 29, 30 or 31.

17. A kit containing: the nucleic acid aptamer molecules according to claim 1, wherein the kit further comprises:
an expression vector contains DNA molecules for transcribing the nucleic acid aptamer molecules according to claim 1; and
a host cell contains the expression vector.

18. Application of the complex according to claim 13 to detection or labeling of complex in vitro or in vivo target nucleic acid molecules, detection or labeling of in vitro or in vivo target molecules, genomic DNA imaging, detection of mRNA and protein contents in cells, detection of genomic DNA, or extraction or purification of RNA.
